(19) United States
(12) Patent Application Publication
(10) Pub. No.: US 2011/0009381 A1
(43) Pub. Date: Jan. 13, 2011

(54) SOLUBILIZED THIAZOLOPYRIDINES

(75) Inventors: Chi B. Vu, Arlington, MA (US); Christopher Oalmann, Watertown, MA (US); Robert B. Perni, Marlborough, MA (US); Jeremy S. Disch, Natick, MA (US); Bruce Szczepankiewicz, Hopkinton, MA (US); Giovanna Gualtieri, Waltham, MA (US); Rebecca L. Casaubon, Chesnut Hill, MA (US)

Correspondence Address: ROPES & GRAY LLP;IPRM - Floor 43, PRUDENTIAL TOWER, 800 BOYLSTON STREET, BOSTON, MA, 02199-3600, US

(73) Assignee: Sirtis Pharmaceuticals, Inc., Cambridge, MA (US)

(21) Appl. No.: 12/742,067

(22) PCT Filed: Nov. 7, 2008
(86) PCT No.: PCT/US08/12548

§ 371 Date: Sep. 28, 2010

Related U.S. Application Data
(60) Provisional application No. 61/002,758, filed on Nov. 8, 2007.

(51) Int. Cl. C07D 513/04; A61K 31/496; A61K 31/5377; A61K 31/4365; A61K 31/541; A61K 31/506; A61K 31/551; A61P 3/10; A61P 3/00

(52) U.S. Cl. 514/210.21; 544/357; 514/252.18; 514/234.2; 544/122; 546/114; 514/228.5; 544/58.2; 514/256; 544/333; 544/328; 544/362; 514/253.04; 514/218; 540/575

(57) ABSTRACT

Provided herein are novel sirtuin-modulating compounds Structural Formula (I): and methods of use thereof. The sirtuin-modulating compounds may be used for increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing as well as diseases or disorders that would benefit from increased mitochondrial activity. Also provided are compositions comprising a sirtuin-modulating compound in combination with another therapeutic agent.

### RELATED APPLICATION

This application claims the benefit of U.S. Provisional Application No. 61/002,758, filed Nov. 8, 2007, the contents of which are incorporated by reference in their entirety.

### BACKGROUND

The Silent Information Regulator (SIR) family of genes represents a highly conserved group of genes present in the genomes of organisms ranging from archaebacteria to a variety of eukaryotes (Frye, 2000). The encoded SIR proteins are involved in diverse processes from regulation of gene silencing to DNA repair. The proteins encoded by members of the SIR gene family show high sequence conservation in a 250 amino acid core domain. A well-characterized gene in this family is S. cerevisiae SIR2, which is involved in silencing HM loci that contain information specifying yeast mating type, telomere position effects and cell aging (Guarente, 1999; Kaeberlein et al., 1999; Shore, 2000). The yeast Sir2 protein belongs to a family of histone deacetylases (reviewed in Guarente, 2000; Shore, 2000). The Sir2 homolog, CobB, in Salmonella typhimurium, functions as an NAD (nicotinamide adenine dinucleotide)-dependent ADP-ribosyl transferase (Tsang and Escalante-Semerena, 1998).

The Sir2 protein is a class III deacetylase which uses NAD as a cosubstrate (Imai et al., 2000; Moazed, 2001; Smith et al., 2000; Tanner et al., 2000; Tanny and Moazed, 2001). Unlike other deacetylases, many of which are involved in gene silencing, Sir2 is insensitive to class I and II histone deacetylase inhibitors like trichostatin A (TSA) (Imai et al., 2000; Landry et al., 2000a; Smith et al., 2000).

Deacetylation of acetyl-lysine by Sir2 is tightly coupled to NAD hydrolysis, producing nicotinamide and a novel acetyl-ADP ribose compound (Tanner et al., 2000; Landry et al., 2000b; Tanny and Moazed, 2001). The NAD-dependent deacetylase activity of Sir2 is essential for its functions which can connect its biological role with cellular metabolism in yeast (Guarente, 2000; Imai et al., 2000; Lin et al., 2000; Smith et al., 2000). Mammalian Sir2 homologs have NAD-dependent histone deacetylase activity (Imai et al., 2000; Smith et al., 2000). Most information about Sir2 mediated functions comes from the studies in yeast (Gartenberg, 2000; Gottschling, 2000).

Biochemical studies have shown that Sir2 can readily deacetylate the amino-terminal tails of histones H3 and H4, resulting in the formation of 1-O-acetyl-ADP-ribose and nicotinamide. Strains with additional copies of SIR2 display increased rDNA silencing and a 30% longer life span. It has recently been shown that additional copies of the C. elegans SIR2 homolog, sir-2.1, and the D. melanogaster dSir2 gene greatly extend life span in those organisms. This implies that the SIR2-dependent regulatory pathway for aging arose early in evolution and has been well conserved. Today, Sir2 genes are believed to have evolved to enhance an organism's health and stress resistance to increase its chance of surviving adversity.

SIRT3 is a homolog of SIRT1 that is conserved in prokaryotes and eukaryotes (P. Onyango et al., Proc. Natl. Acad. Sci. USA 99: 13653-13658 (2002)). The SIRT3 protein is targeted to the mitochondrial cristae by a unique domain located at the N-terminus. SIRT3 has NAD+-dependent protein deacetylase activity and is upbiquitously expressed, particularly in metabolically active tissues. Upon transfer to the mitochondria, SIRT3 is believed to be cleaved into a smaller, active form by a mitochondrial matrix processing peptidase (MPP) (B. Schwer et al., J. Cell Biol. 158: 647-657 (2002)).

Caloric restriction has been known for over 70 years to improve the health and extend the lifespan of mammals (Masoro, 2000). Yeast life span, like that of metazoans, is also extended by interventions that resemble caloric restriction, such as low glucose. The discovery that both yeast and flies lacking the SIR2 gene do not live longer when calorically restricted provides evidence that SIR2 genes mediate the beneficial health effects of this diet (Anderson et al., 2003; Helfand and Rogina, 2004). Moreover, mutations that reduce the activity of the yeast glucose-responsive cAMP (adenosine 3′,5′-monophosphate)-dependent (PKA) pathway extend life span in wild type cells but not in mutant sir2 strains, demonstrating that SIR2 is likely to be a key downstream component of the caloric restriction pathway (Lin et al., 2001).

### SUMMARY

Provided herein are novel sirtuin-modulating compounds and methods of use thereof.

In one aspect, the invention provides sirtuin-modulating compounds of Structural Formulas (I) and (II) as are described in detail below.

In another aspect, the invention provides methods for using sirtuin-modulating compounds, or compostions comprising sirtuin-modulating compounds. In certain embodiments, sirtuin-modulating compounds that increase the level and/or activity of a sirtuin protein may be used for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, chemotherapeutic induced neuropathy, neuropathy associated with an ischemic event, ocular diseases and/or disorders, cardiovascular disease, blood clotting disorders, inflammation, and/or flushing, etc. Sirtuin-modulating compounds that increase the level and/or activity of a sirtuin protein may also be used for treating a disease or disorder in a subject that would benefit from increased mitochondrial activity, for enhancing muscle performance, for increasing muscle ATP levels, or for treating or preventing muscle tissue damage associated with hypoxia or ischemia. In other embodiments, sirtuin-modulating compounds that decrease the level and/or activity of a sirtuin protein may be used for a variety of therapeutic applications including, for example, increasing cellular sensitivity to stress, increasing apoptosis, treatment of cancer, stimulation of appetite, and/or stimulation of weight gain, etc. As described further below, the methods comprise administering to a subject in need thereof a pharmaceutically effective amount of a sirtuin-modulating compound.

In certain aspects, the sirtuin-modulating compounds may be administered alone or in combination with other compounds, including other sirtuin-modulating compounds, or other therapeutic agents.

### DETAILED DESCRIPTION

### 1. Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

“Biologically active portion of a sirtuin” refers to a portion of a sirtuin protein having a biological activity, such as the ability to deacetylate. Biologically active portions of a sirtuin may comprise the core domain of sirtuins. Biologically active portions of SIRT1 having GenBank Accession No. NP_{—}036370 that encompass the NAD+ binding domain and the substrate binding domain, for example, may include without limitation, amino acids 62-293 of GenBank Accession No. NP_{—}036370, which are encoded by nucleotides 237 to 932 of GenBank Accession No. NM_{—}012238. Therefore, this region is sometimes referred to as the core domain. Other biologically active portions of SIRT1, also sometimes referred to as core domains, include about amino acids 261 to 447 of GenBank Accession No. NP_{—}036370, which are encoded by nucleotides 834 to 1394 of GenBank Accession No. NM_{—}012238; about amino acids 242 to 493 of GenBank Accession No. NP_{—}036370, which are encoded by nucleotides 777 to 1532 of GenBank Accession No. NM_{—}012238; or about amino acids 254 to 495 of GenBank Accession No. NP_{—}036370, which are encoded by nucleotides 813 to 1538 of GenBank Accession No. NM_{—}012238.

The term “companion animals” refers to cats and dogs. As used herein, the term “dog(s)” denotes any member of the species Canis familiaris, of which there are a large number of different breeds. The term “cat(s)” refers to a feline animal including domestic cats and other members of the family Felidae, genus Felis.

“Diabetes” refers to high blood sugar or ketoacidosis, as well as chronic, general metabolic abnormalities arising from a prolonged high blood sugar status or a decrease in glucose tolerance. “Diabetes” encompasses both the type I and type II (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

A “direct activator” of a sirtuin is a molecule that activates a sirtuin by binding to it. A “direct inhibitor” of a sirtuin is a molecule inhibits a sirtuin by binding to it.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “hyperinsulinemia” refers to a state in an individual in which the level of insulin in the blood is higher than normal.

The term “insulin resistance” refers to a state in which a normal amount of insulin produces a subnormal biologic response relative to the biological response in a subject that does not have insulin resistance.

An “insulin resistance disorder,” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

The term “livestock animals” refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus.

The term “mammal” is known in the art, and exemplary mammals include humans, primates, livestock animals (including bovines, porcines, etc.), companion animals (e.g., canines, felines, etc.) and rodents (e.g., mice and rats).

“Obese” individuals or individuals suffering from obesity are generally individuals having a body mass index (BMI) of at least 25 or greater. Obesity may or may not be associated with insulin resistance.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

A “patient”, “subject”, “individual” or “host” refers to either a human or a non-human animal.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “pyrogen-free”, with reference to a composition, refers to a composition that does not contain a pyrogen in an amount that would lead to an adverse effect (e.g., irritation, fever, inflammation, diarrhea, respiratory distress, endotoxic shock, etc.) in a subject to which the composition has been administered. For example, the term is meant to encompass compositions that are free of, or substantially free of, an endotoxin such as, for example, a lipopolysaccharide (LPS).

“Replicative lifespan” of a cell refers to the number of daughter cells produced by an individual “mother cell.” “Chronological aging” or “chronological lifespan,” on the other hand, refers to the length of time a population of non-dividing cells remains viable when deprived of nutrients. “Increasing the lifespan of a cell” or “extending the lifespan of a cell,” as applied to cells or organisms, refers to increasing the number of daughter cells produced by one cell; increasing the ability of cells or organisms to cope with stresses and combat damage, e.g., to DNA, proteins; and/or increasing the ability of cells or organisms to survive and exist in a living state for longer under a particular condition, e.g., stress (for example, heatshock, osmotic stress, high energy radiation, chemically-induced stress, DNA damage, inadequate salt level, inadequate nitrogen level, or inadequate nutrient level). Lifespan can be increased by at least about 20%, 30%, 40%, 50%, 60% or between 20% and 70%, 30% and 60%, 40% and 60% or more using methods described herein.

“Sirtuin-activating compound” refers to a compound that increases the level of a sirtuin protein and/or increases at least one activity of a sirtuin protein. In an exemplary embodiment, a sirtuin-activating compound may increase at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells.

“Sirtuin-inhibiting compound” refers to a compound that decreases the level of a sirtuin protein and/or decreases at least one activity of a sirtuin protein. In an exemplary embodiment, a sirtuin-inhibiting compound may decrease at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells.

“Sirtuin-modulating compound” refers to a compound of Structural Formulas (I) and (II) as described herein. In exemplary embodiments, a sirtuin-modulating compound may either up regulate (e.g., activate or stimulate), down regulate (e.g., inhibit or suppress) or otherwise change a functional property or biological activity of a sirtuin protein. Sirtuin-modulating compounds may act to modulate a sirtuin protein either directly or indirectly. In certain embodiments, a sirtuin-modulating compound may be a sirtuin-activating compound or a sirtuin-inhibiting compound.

“Sirtuin protein” refers to a member of the sirtuin deacetylase protein family, or preferably to the sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}012237, NM_{—}030593, NP_{—}036369, NP_{—}085096, and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

“SIRT1 protein” refers to a member of the sir2 family of sirtuin deacetylases. In one embodiment, a SIRT1 protein includes yeast Sir2 (GenBank Accession No. P53685), C. elegans Sir-2.1 (GenBank Accession No. NP_{—}501912), human SIRT1 (GenBank Accession No. NM_{—}012238 or NP_{—}036370 (or AF083106)), and human SIRT2 (GenBank Accession No. NM_{—}012237, NM_{—}030593, NP_{—}036369, NP_{—}085096, or AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP_{—}036370, NP_{—}501912, NP_{—}085096, NP_{—}036369, or P53685. SIRT1 proteins include polypeptides comprising all or a portion of the amino acid sequence set forth in GenBank Accession Nos. NP_{—}036370, NP 501912, NP_{—}085096, NP_{—}036369, or P53685; the amino acid sequence set forth in GenBank Accession Nos. NP_{—}036370, NP 501912, NP_{—}085096, NP_{—}036369, or P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP_{—}036369, or P53685, and functional fragments thereof. Polypeptides of the invention also include homologs (e.g., orthologs and paralogs), variants, or fragments, of GenBank Accession Nos. NP_{—}036370, NP_{—}501912, NP_{—}085096, NP_{—}036369, or P53685.

“SIRT3 protein” refers to a member of the sirtuin deacetylase protein family and/or to a homolog of a SIRT1 protein. In one embodiment, a SIRT3 protein includes human SIRT3 (GenBank Accession No. AAH01042, NP_{—}036371, or NP_{—}001017524) and mouse SIRT3 (GenBank Accession No. NP_{—}071878) proteins, and equivalents and fragments thereof. In another embodiment, a SIRT3 protein includes a polypeptide comprising a sequence consisting of or consisting essentially of, the amino acid sequence set forth in GenBank Accession Nos. AAH01042, NP_{—}036371, NP_{—}001017524, or NP_{—}071878. SIRT3 proteins include polypeptides comprising all or a portion of the amino acid sequence set forth in GenBank Accession AAH01042, NP_{—}036371, NP_{—}001017524, or NP_{—}071878; the amino acid sequence set forth in GenBank Accession Nos. AAH01042, NP_{—}036371, NP_{—}001017524, or NP_{—}071878 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. AAH01042, NP_{—}036371, NP_{—}001017524, or NP_{—}071878, and functional fragments thereof. Polypeptides of the invention also include homologs (e.g., orthologs and paralogs), variants, or fragments, of GenBank Accession Nos. AAH01042, NP_{—}036371, NP_{—}001017524, or NP_{—}071878. In one embodiment, a SIRT3 protein includes a fragment of SIRT3 protein that is produced by cleavage with a mitochondrial matrix processing peptidase (MPP) and/or a mitochondrial intermediate peptidase (MIP).

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect at a reasonable benefit/risk ratio applicable to such treatment.

“Treating” a condition or disease refers to curing as well as ameliorating at least one symptom of the condition or disease.

The term “vision impairment” refers to diminished vision, which is often only partially reversible or irreversible upon treatment (e.g., surgery). Particularly severe vision impairment is termed “blindness” or “vision loss”, which refers to a complete loss of vision, vision worse than 20/200 that cannot be improved with corrective lenses, or a visual field of less than 20 degrees diameter (10 degrees radius).

### 2. Sirtuin Modulators

In one aspect, the invention provides novel sirtuin-modulating compounds for treating and/or preventing a wide variety of diseases and disorders including, for example, diseases or disorders related to aging or stress, diabetes, obesity, neurodegenerative diseases, ocular diseases and disorders, cardiovascular disease, blood clotting disorders, inflammation, cancer, and/or flushing, etc. Sirtuin-modulating compounds that increase the level and/or activity of a sirtuin protein may also be used for treating a disease or disorder in a subject that would benefit from increased mitochondrial activity, for enhancing muscle performance, for increasing muscle ATP levels, or for treating or preventing muscle tissue damage associated with hypoxia or ischemia. Other compounds disclosed herein may be suitable for use in a pharmaceutical composition and/or one or more methods disclosed herein.

In one embodiment, sirtuin-modulating compounds of the invention are represented by Structural Formula (I):

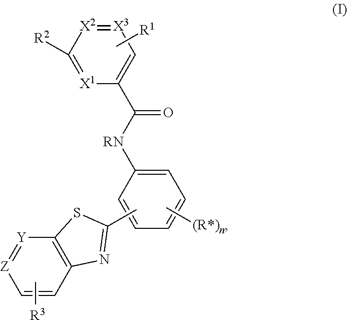

or a salt thereof, wherein:
two of X¹, X² and X³ are independently selected from —CH— and —N—;

the other of X¹, X² and X³ is —CH—;

R¹ is a solubilizing group;

R² is selected from phenyl, lower alkyl phenyl, fluorophenyl and a 5- to 6-membered heterocycle containing an N heteroatom and, optionally, a second heteroatom selected from N, O or S, wherein said heterocycle is optionally substituted with methyl;

R is —H or —CH₃;

one of Y and Z is —CH— and the other of Y and Z is —N—;

R³ is selected from hydrogen, halo, lower alkyl, lower alkoxy, lower alkylthio and lower alkylsulfonyl;

R* is —CH₃ or a halogen; and

n is an integer from 0-4.

In certain embodiments, sirtuin-modulating compounds of the invention are represented by Structural Formula (II):

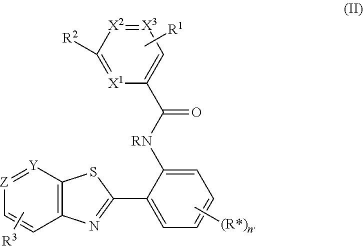

The following values apply to both Structural Formulas (I) and (II).

In certain embodiments, X¹ is —N—. In certain embodiments, X² is —N—. In certain embodiments, X³ is —N—. In certain embodiments, X¹ and X² are —N— and X³ is —CH—.

In certain embodiments, R² is selected from substituted or unsubstituted: phenyl, thiazolyl, pyrimidinyl, pyridyl and pyrazolyl. In certain embodiments, R² is selected from phenyl, lower alkyl phenyl, fluorophenyl, methylthiazolyl, pyrimidinyl, pyridyl and pyrazolyl. In certain such embodiments, R² is selected from phenyl, lower alkyl phenyl such as methyl phenyl, fluorophenyl, 2-methylthiazol-4-yl, pyridyl and pyrazol-1-yl. Typically, R² is phenyl, lower alkyl phenyl or pyridyl.

In certain embodiments, Y is —N— and Z is —CH—. In other embodiments, Z is —N— and Z is —CH—. In certain embodiments, wherein Y is —N— and Z is —CH—, R² is selected from phenyl, lower alkyl phenyl such as methyl phenyl, 3-fluorophenyl and pyridyl and X¹ and X² are —N— and X³ is —CH—.

In certain embodiments, R³ is selected from hydrogen, halo, lower alkyl, lower alkoxy, lower alkylthio and lower alkylsulfonyl. In certain embodiments, R³ is hydrogen. In particular embodiments, X¹ and X² are —N—, X³ is —CH—, R² is selected from phenyl, lower alkyl phenyl, 3-fluorophenyl and pyridyl and R³ is selected from hydrogen, halo, lower alkyl, and lower alkoxy.

In certain embodiments, R¹ is —NR⁴R⁵ and R⁴ and R⁵ are each independently selected from hydrogen or lower alkyl. In certain embodiments, R⁴ is lower alkyl, amino lower alkyl, lower alkyl amino lower alkyl, lower dialkyl amino lower alkyl, monocyclyl lower alkyl, monocyclyl amino lower alkyl, or monocyclyl, and R⁵ is lower alkyl or H. In particular embodiments, monocyclyl is a nitrogen-containing monocycle. In particular embodiments, R² is selected from phenyl, lower alkyl phenyl, 3-fluorophenyl and pyridyl, X¹ and X² are —N—, X³ is —CH—, and R¹ is —NHR⁴ wherein R⁴ is lower alkyl, amino lower alkyl, alkyl amino lower alkyl, or lower dialkyl amino lower alkyl.

In certain embodiments, R¹ is a nitrogen-containing monocycle. In certain embodiments, R¹ is a nitrogen-containing monocycle where the point of attachment is an annular nitrogen. In certain embodiments, the nitrogen-containing monocycle is a 4, 5, 6, 7, or 8-membered heterocycle. In certain embodiments, the heterocycle is a 5, 6 or 7-membered heterocycle. In certain embodiments, the nitrogen-containing heterocycle is substituted or unsubstituted thiazolyl, oxazolyl, isoxazolyl, isothiozolyl, triazolyl, tetrazolyl, pyrazolyl, imidazolyl, pyridinyl, pyrrolyl, thiazinyl, oxazinyl, piperidinyl, piperazinyl, pyrimidinyl, morpholinyl, thiomorpholinyl and 1,1-dioxo-1-thiomorpholinyl. In particular embodiments, R² is selected from phenyl, lower alkyl phenyl, 3-fluorophenyl and pyridyl, X′ and X² are —N—, X³ is —CH—, and R¹ is a nitrogen-containing monocycle wherein the point of attachment is an annular nitrogen.

In certain embodiments, R¹ is represented by:

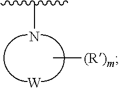

wherein the monocycle is a 5, 6 or 7-membered heterocycle; W is —N(R⁶)—, —S(O₂)—, —C(R⁶R⁶)—, —N(CO₂R⁶)—, —O— or —S—; R¹ in each occurrence is independently selected from H, lower alkyl carbonyl, lower alkyl carboxy, lower alkylcarbonyloxy, lower alkyl amino carbonyl, lower alkylcarbonyl amino, and lower alkyl; m is 0 to 2; and each R⁶ is independently selected from H and lower alkyl. In particular embodiments, R¹ is represented by:

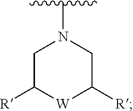

R² is selected from phenyl, lower alkyl phenyl, 3-fluorophenyl and pyridyl, X¹ and X² are —N—, and X³ is —CH—.

In certain embodiments, R¹ is a nitrogen-containing heterocycle where the point of attachment is an annular nitrogen. In certain embodiments, the heterocycle comprises 2 rings, such as a bridged or a fused heterocycle. In certain embodiments, R¹ is selected from a 6,6-, (e.g., 1,2,3,4-tetrahydroquinoline) or 6,5- (e.g., indole) fused nitrogen-containing heterocycles. In particular embodiments, R¹ is represented by:

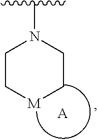

wherein M is —CH— or —N— and ring A is 5- or 6-membered. In certain embodiments, ring A is 5-membered and M is —N—.

In certain embodiments, R¹ is represented by:

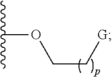

wherein G is —NR⁴R⁵, —SR⁶, —OR⁶, —SO₂R⁶, —NCO₂R⁶—NR⁴SO₂R⁶ or monocyclyl; p is 0 to 3; v is 0 to 2; R⁴ is lower alkyl, monocyclyl amino or monocyclyl lower alkyl and R⁵ is lower alkyl or H; each R⁶ is independently H or lower alkyl, and R¹ is optionally substituted by one or more substituents independently selected from oxo, carbonyl, carboxy, lower alkyl carboxy, lower alkyl, hydroxyl, thio, halo, monocyclyl or cyano. Exemplary monocyclyl groups include substituted or unsubstituted morpholinyl, thiomorpholinyl, piperidinyl, pyrimidinyl, 1,1-dioxo-1-thiomorpholinyl, thiazolyl and oxazolyl. In particular embodiments, R¹ is represented by:

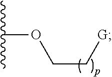

R² is selected from phenyl, lower alkyl phenyl, 3-fluorophenyl and pyridyl, X¹ and X² are —N—, and X³ is —CH—.

In certain embodiments, R¹ is —(CH₂)ₖG, and G is —NR⁴R⁵, —SR⁶, —OR⁶, —SO₂R⁶, —NCO₂R⁶ or monocyclyl; k is 1 to 3; R⁴ is lower alkyl, monocyclyl amino or monocyclyl lower alkyl and R⁵ is lower alkyl or H; and each R⁶ is independently H or lower alkyl. Exemplary monocyclyl groups include those mentioned above. In particular embodiments, R² is selected from phenyl, lower alkyl phenyl, 3-fluorophenyl and pyridyl, X¹ and X² are —N—, X³ is —CH—, and R¹ is —(CH₂)ₖG, and G is —NR⁴R⁵, —SR⁶, —OR⁶, —SO₂R⁶, —NCO₂R⁶ or monocyclyl.

In certain embodiments, R¹ is selected from a moiety containing at least two heteroatoms. In certain such embodiments, one of the at least two heteroatoms of R¹ is a nitrogen. In certain embodiments, R¹ comprises at least two heteroatoms, one of which is a nitrogen, and a monocycle.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

Sirtuin-modulating compounds of the invention advantageously modulate the level and/or activity of a sirtuin protein, particularly the deacetylase activity of the sirtuin protein.

Separately or in addition to the above properties, certain sirtuin-modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity, at concentrations of the compound that are effective for modulating the deacetylation activity of a sirtuin protein (e.g., such as a SIRT1 and/or a SIRT3 protein).

An alkyl group is a straight chained or branched non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl.

Lower alkyl is a straight or branched alkyl group containing from 1-8 carbon atoms, such as methyl, ethyl, n-propyl, iso-propyl, n-butyl, sec-butyl, tert-butyl, pentyl, hexyl, pentyl, octyl and the like. Optionally, lower alkyl is substituted with one or more subtituents selected from halo, cyano, amino, hydroxyl, thio, carbonyl, oxo, lower alkyl carbonyl, lower alkoxy, lower alkyl thio, lower alkylcarbonyloxy, monocyclyl, carboxy, lower alkyl carboxy, lower alkyl sulfonyl, lower alkylamino and lower dialkylamino.

A cycloalkyl group is a cyclic alkyl group.

Alkenyl and alkynyl groups are analogous to alkyl, but contain one or more double or triple bonds, respectively.

Monocyclyl includes 5-7 membered aryl or heteroaryl, 3-7 membered cycloalkyl, and 5-7 membered non-aromatic heterocyclyl. Monocyclyl is optionally substituted with one or more substituents selected from halo, cyano, amino, hydroxyl, thio, carbonyl, oxo, lower alkyl, lower alkoxy, lower alkyl thio, lower alkylcarbonyloxy, lower alkyl carboxy, lower alkoxy lower alkyl, lower alkylcarbonyl, monocyclyl carbonyl, arylcarbonyl, aryloxy, monocyclyloxy, lower alkylsulfonyl, hydroxycarbonyl, cyclopropyl, lower alkyl thio, lower alkylsulfinyl, lower alkylsulfonyl, lower alkylamino, lower dialkylamino, monocyclyl (e.g cycloalkyl, pyridyl, phenyl), monocyclyl lower alkyl, aminocarbonyl, lower alkyl-aminocarbonyl, di (lower alkyl)-aminocarbonyl, aminoalkylaminocarbonyl, lower alkyl-aminoalkylaminocarbonyl, di(lower alkyl)-aminoalkylaminocarbonyl, amino, sulfonamido, lower alkyl sulfonamido, cyclic amino (including monocyclic and fused bicyclic amino, e.g., morpholino, pyrrolidinyl, piperadinyl, piperazinyl, octahydropyrrolo[1,2-a]pyrazin-2-yl), cyclic aminocarbonyl (e.g, morpholinocarbonyl, pyrrolidinylcarbonyl, piperadinylcarbonyl, piperazinylcarbonyl), cyclic amino-carbonylamino (e.g, morpholinocarbonylamino, pyrrolidinylcarbonylamino, piperadinylcarbonylamino, piperazinylcarbonylamino), cyclic ethers (e.g., tetrahydrofuranyl, tetrahydropyranyl), and halo(tetrahydropyranylidene) lower alkyl (e.g., fluoro(4-tetrahydropyranylidene)methyl), along with solubilizing groups other than those specificially named above, particularly cyclic solubilizing groups. Exemplary monocyclyl groups include substituted or unsubstituted heterocycles such as thiazolyl, oxazolyl, oxazinyl, thiazinyl, thiadiazolyl, dithianyl, dioxanyl, isoxazolyl, isothiozolyl, triazolyl, furanyl, tetrahydrofuranyl, dihydrofuranyl, pyranyl, tetrazolyl, pyrazolyl, pyrazinyl, pyridazinyl, imidazolyl, pyridinyl, pyrrolyl, dihydropyrrolyl, pyrrolidinyl, thiazinyl, oxazinyl, piperidinyl, piperazinyl, pyrimidinyl, morpholinyl, tetrahydrothiophenyl, thiophenyl, cyclohexyl, cyclopentyl, cyclopropyl, cyclobutyl, cycloheptanyl, azetidinyl, oxetanyl, thiiranyl, oxiranyl, aziridinyl, and thiomorpholinyl.

Heterocyclic includes 4-7 membered monocyclic and 8-12 membered bicyclic rings comprising one or more heteroatoms selected from, for example, N, O, and S atoms. In certain embodiments, the heterocyclic group is selected from saturated, unsaturated or aromatic. A heterocycle is optionally substituted with one or more substituents selected from halo, cyano, amino, hydroxyl, thio, carbonyl, oxo, lower alkyl, lower alkoxy, lower alkyl thio, lower alkylcarbonyloxy, lower alkyl carboxy, lower alkoxy lower alkyl, lower alkylcarbonyl, monocyclyl carbonyl, arylcarbonyl, aryloxy, monocyclyloxy, lower alkylsulfonyl, hydroxycarbonyl, cyclopropyl, lower alkyl thio, lower alkylsulfinyl, lower alkylsulfonyl, lower alkylamino, lower dialkylamino, monocyclyl (e.g cycloalkyl, pyridyl, phenyl), monocyclyl lower alkyl, aminocarbonyl, lower alkyl-aminocarbonyl, di (lower alkyl)-aminocarbonyl, aminoalkylaminocarbonyl, lower alkyl-aminoalkylaminocarbonyl, di(lower alkyl)-aminoalkylaminocarbonyl, amino, sulfonamido, lower alkyl sulfonamido, cyclic amino (including monocyclic and fused bicyclic amino, e.g., morpholino, pyrrolidinyl, piperadinyl, piperazinyl, octahydropyrrolo[1,2-a]pyrazin-2-yl), cyclic aminocarbonyl (e.g, morpholinocarbonyl, pyrrolidinylcarbonyl, piperadinylcarbonyl, piperazinylcarbonyl), cyclic amino-carbonylamino (e.g, morpholinocarbonylamino, pyrrolidinylcarbonylamino, piperadinylcarbonylamino, piperazinylcarbonylamino), cyclic ethers (e.g., tetrahydropyranyl, tetrahydropyranyl), and halo(tetrahydropyranylidene) lower alkyl (e.g., fluoro(4-tetrahydropyranylidene)methyl), along with solubilizing groups other than those specificially named above, particularly cyclic solubilizing groups.

Aromatic (aryl) groups include carbocyclic aromatic groups such as phenyl, naphthyl, and anthracyl, and heteroaryl groups such as imidazolyl, thienyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Suitable substituents on an alkyl, alkenyl, alkynyl, monocyclyl or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of generally suitable substituents include —OH, halogen (—Br, —Cl, —I and —F), —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(WW), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}-R^{d} are each independently an optionally substituted group selected from an aliphatic, benzyl, or aromatic group, preferably an alkyl, benzylic or aryl group. Optional substituents on R^{a}-R^{d} are selected from NH₂, NH(C₁₋₄aliphatic), N(C₁₋₄aliphatic)₂, halogen, C₁₋₄oliphatic, OH, O(C₁₋₄aliphatic), NO₂, CN, CO₂H, CO₂(C₁₋₄oliphatic), O(haloC₁₋₄ aliphatic), or haloC₁₋₄aliphatic, wherein each of the foregoing C₁₋₄oliphatic groups of is unsubstituted. In addition, —NR^{a}R^{b}, taken together, can also form a substituted or unsubstituted non-aromatic heterocyclic group. A non-aromatic heterocyclic group, or aryl group can also have an aliphatic or substituted aliphatic group as a substituent. A substituted aliphatic group can also have a non-aromatic heterocyclic ring, a substituted a non-aromatic heterocyclic ring, aryl or substituted aryl group as a substituent. A substituted aliphatic, non-aromatic heterocyclic group, substituted aryl, or substituted benzyl group can have more than one substituent.

Generally suitable substituents on an aryl ring are selected from a solubilizing group, halogen; —R^{o}; —OR^{o}; —SR^{o}; 1,2-methylenedioxy; 1,2-ethylenedioxy; phenyl (Ph) optionally substituted with R^{o}; —O(Ph) optionally substituted with R^{o}; —(CH₂)₁₋₂(Ph), optionally substituted with R^{o}; —CH═CH(Ph), optionally substituted with R^{o}; —NO₂; —CN; —N(R^{o})₂; —C(O)C(O)R^{o}; —C(O)CH₂C(O)R^{o}; —CO₂R^{o}; —C(O)R^{o}; —S(O)₂R^{o}; —SO₂N(R^{o})₂; —S(O)R^{o}; —NR^{o}SO₂N(R^{o})₂; —NR^{o}SO₂R^{o}; —C(═S)N(R^{o})₂; or —C(═NH)—N(R^{o})₂; or wherein each independent occurrence of R^{o} is selected from hydrogen, optionally substituted C₁₋₆ aliphatic, an unsubstituted 5-6 membered heteroaryl or heterocyclic ring, phenyl, —O(Ph), or —CH₂(Ph), or, notwithstanding the definition above, two independent occurrences of R^{o}, on the same substituent or different substituents, taken together with the atom(s) to which each R^{o} group is bound, form a 3-8-membered cycloalkyl, heterocyclyl, aryl, or heteroaryl ring having 0-3 heteroatoms independently selected from nitrogen, oxygen, or sulfur. Optional substituents on the aliphatic group of R^{o} are selected from NH₂, NH(C₁₋₄aliphatic), N(C₁₋₄oliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, wherein each of the foregoing C₁₋₄aliphatic groups of R^{o} is unsubstituted

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

As used herein, a “solubilizing group” is a moiety that has hydrophilic character sufficient to improve or increase the water-solubility of the compound in which it is included, as compared to an analog compound that does not include the group. The hydrophilic character can be achieved by any means, such as by the inclusion of functional groups that ionize under the conditions of use to form charged moieties (e.g., carboxylic acids, sulfonic acids, phosphoric acids, amines, etc.); groups that include permanent charges (e.g., quaternary ammonium groups); and/or heteroatoms or heteroatomic groups (e.g., O, S, N, NH, N—(CH₂)_{y}—R^{a}, N—(CH₂)_{y}—C(O)R^{a}, N—(CH₂)_{y}—C(O)OR^{a}, N—(CH₂)_{y}—S(O)₂R^{a}—, N—(CH₂)_{y}—S(O)₂OR^{a}, N—(CH₂)_{y}—C(O)NR^{a}R^{a}, etc., wherein R^{a} is selected from hydrogen, lower alkyl, lower cycloalkyl, (C₆-C₁₄) aryl, phenyl, naphthyl, (C₇-C₂₀) arylalkyl and benzyl, wherein R^{a} is optionally substituted; and y is an integer ranging from 0 to 6), optionally substituted heterocyclic groups (e.g., —(CH₂)ₙ—R^{b}, —(CH₂)ₙ—C(O)—R^{b}, —(CH₂)ₙ—O—(CH₂)ₙ—R^{b}, wherein R^{b} is selected from an optionally substituted saturated monocyclic heterocycle, an optionally substituted saturated bicyclic fused heterocycle, an optionally substituted saturated bicyclic spiro heterocycle, an optionally substituted heteroaryl and an optionally substituted partially substituted non-aryl heterocycle; and n is an integer ranging from 0 to 2). It should be understood that substituents present on R^{a} or R^{b} need not improve or increase water solubility over their unsubstituted counterparts to be within the scope of this definition. All that is required is that such substituents do not significantly reverse the improvement in water-solubility afforded by the unsubstituted R^{a} or R^{b} moiety.

In one embodiment, the solubilizing group increases the water-solubility of the corresponding compound lacking the solubilizing group at least 5-fold, preferably at least 10-fold, more preferably at least 20-fold and most preferably at least 50-fold.

In one preferred embodiment, the solubilizing group is a moiety of the formula:

—(CH₂)ₙ—R¹⁰⁰—N(R¹⁰¹)(R¹⁰¹), wherein:

n is selected from 0, 1 or 2;
R¹⁰⁰ is selected from a bond, —C(O)—, or —O(CH₂)ₙ; and
each R¹⁰¹ is independently selected from:
a. hydrogen;
b. C₁-C₄ straight or branched alkyl, wherein said alkyl is optionally substituted with halo, CN, OH, O—(C₁-C₄ straight or branched alkyl), N(R₁′)(R₁′), or ═O;

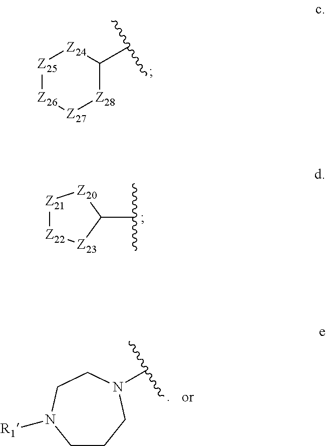

f. both R¹⁰¹ moieties are taken together with the nitrogen atom to which they are bound to form a ring of the structure

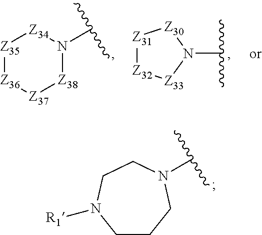

or
g. both R¹⁰¹ moieties are taken together with the nitrogen atom to which they are bound to form a 5-membered heteroaryl ring containing 1 to 3 additional N atoms, wherein said heteroaryl ring is optionally substituted with R₁′;
   wherein:

each Z is independently selected from —O—, —S—, —NR₁′—, or —C(R⁵⁰)(R⁵⁰)—, wherein:
at least three of Z₂₀, Z₂₁, Z₂₂, and Z₂₃ are —C(R⁵⁰)(R⁵⁰)—;
at least three of Z₂₄, Z₂₅, Z₂₆, Z₂₇, and Z₂₈ are —C(R⁵⁰)(R⁵⁰)—;
at least four of Z₃₀, Z₃₁, Z₃₂, and Z₃₃ are —C(R⁵⁰)(R⁵⁰)—; and
at least four of Z₃₄, Z₃₅, Z₃₆, Z₃₇, and Z₃₈ are —C(R⁵⁰)(R⁵⁰)—;

each R₁′ is independently selected from hydrogen or a C₁-C₃ straight or branched alkyl optionally substituted with one or more substituent independently selected from halo, —CN, —OH, —OCH₃, —NH₂, —NH(CH₃), —N(CH₃)₂, or ═O;

each R⁵⁰ is independently selected from R₁′, halo, CN, OH, O—(C₁-C₄ straight or branched alkyl), N(R₁′)(R₁′), ═CR₁′, SR₁′, ═NR₁′, ═NOR₁′, or ═O;

any two suitable non-cyclic R⁵⁰ are optionally bound to one another directly or via a C₁ to C₂ alkylene, alkenylene or alkanediylidene bridge to produce a bicyclic fused or spiro ring; and

any

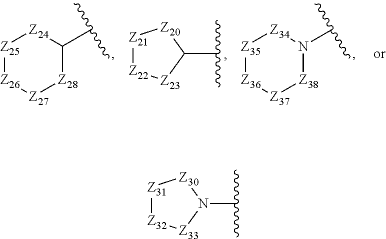

ring structure is optionally benzofused or fused to a monocyclic heteroaryl to produce a bicyclic ring.

For clarity, the term “C₁ to C₂ alkylene, alkenylene or alkanediylidene bridge” means the multivalent structures —CH₂—, —CH₂—CH₂—, —CH═, ═CH—, —CH═CH—, or ═CH—CH═. The two R⁵⁰ moieties that are optionally bound to one another can be either on the same carbon atom or different carbon atoms. The former produces a spiro bicyclic ring, while the latter produces a fused bicyclic ring. It will be obvious to those of skill in the art that when two R⁵⁰ are bound to one another to form a ring (whether directly or through one of the recited bridges), one or more terminal hydrogen atoms on each R⁵⁰ will be lost. Accordingly, a “suitable non-cyclic R⁵⁰” moiety available for forming a ring is a non-cyclic R⁵⁰ that comprises at least one terminal hydrogen atom.

In another embodiment, the solubilizing group is a moiety of the formula: —(CH₂)ₙ—O—R¹⁰¹, wherein n and R¹⁰¹ are as defined above.

In yet another embodiment, the solubilizing group is a moiety of the formula: —(CH₂)ₙ—C(O)—R₁′, wherein n and R₁′ are as defined above.

In certain embodiments, a solubilizing group is selected from —(CH₂)ₙ—R¹⁰², wherein n is 0, 1 or 2, preferably 2; and R¹⁰² is selected from

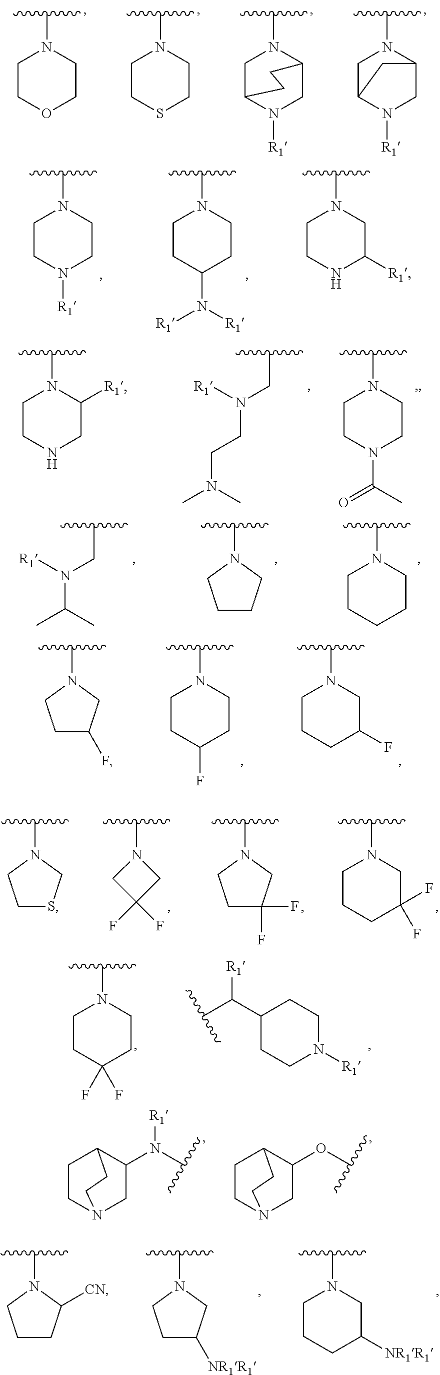

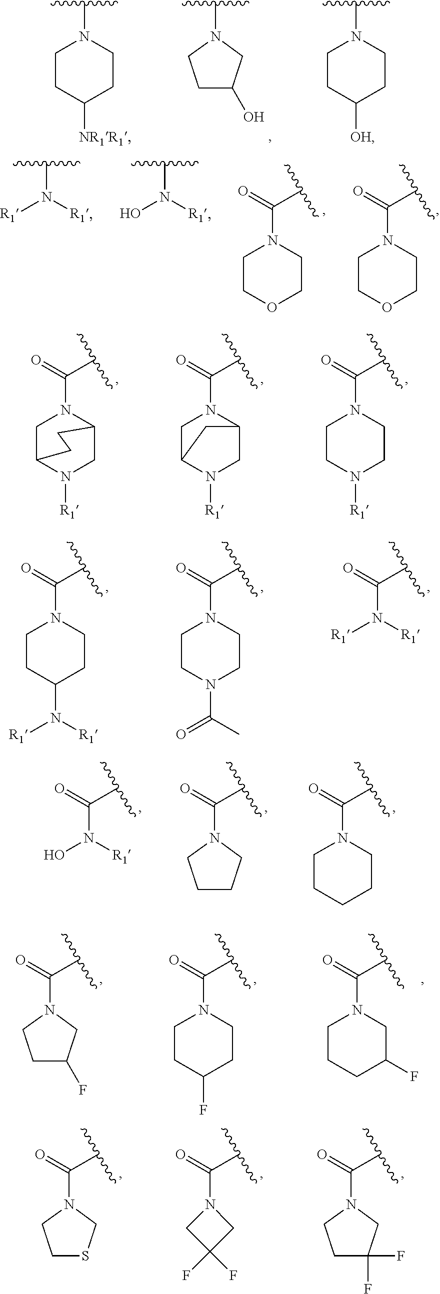

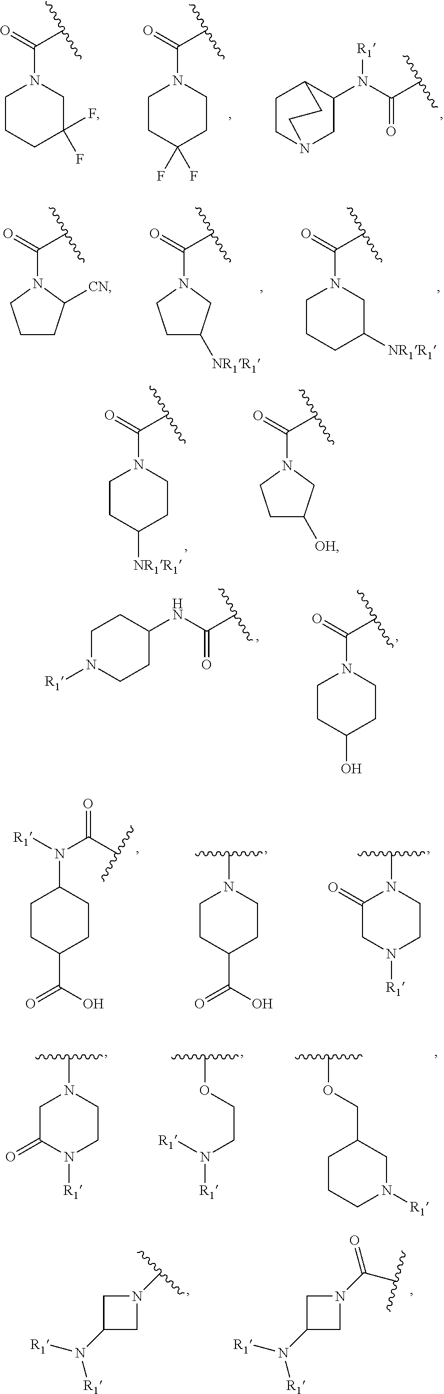

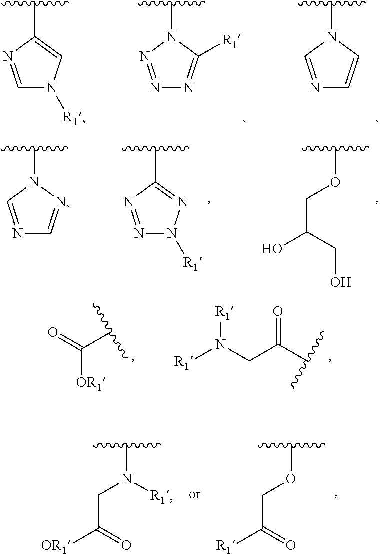

wherein R₁′ groups are as defined above.

In certain particular embodiments, a solubilizing group is selected from 2-dimethylaminoethylcarbamoyl, piperazin-1-ylcarbonyl, piperazinylmethyl, dimethylaminomethyl, 4-methylpiperazin-1-ylmethyl, 4-aminopiperidin-1-yl-methyl, 4-fluoropiperidin-1-yl-methyl, morpholinomethyl, pyrrolidin-1-ylmethyl, 2-oxo-4-benzylpiperazin-1-ylmethyl, 4-benzylpiperazin-1-ylmethyl, 3-oxopiperazin-1-ylmethyl, piperidin-1-ylmethyl, piperazin-1-ylethyl, 2,3-dioxopropylaminomethyl, thiazolidin-3-ylmethyl, 4-acetylpiperazin-1-ylmethyl, 4-acetylpiperazin-1-yl, morpholino, 3,3-difluoroazetidin-1-ylmethyl, 2H-tetrazol-5-ylmethyl, thiomorpholin-4-ylmethyl, 1-oxothiomorpholin-4-ylmethyl, 1,1-dioxothiomorpholin-4-ylmethyl, 1H-imidazol-1-ylmethyl, 3,5-dimethylpiperazin-1-ylmethyl, 4-hydroxypiperidin-1-ylmethyl, N-methyl(1-acetylpiperidin-4-yl)-aminomethyl, N-methylquinuclidin-3-ylaminomethyl, 1H-1,2,4-triazol-1-ylmethyl, 1-methylpiperidin-3-yl-oxymethyl, or 4-fluoropiperidin-1-yl.

To the extent not included within any of the definitions set forth above, the term “solubilizing group” also includes moieties disclosed as being attached to the 7-position of 1-cyclopropyl-6-fluoro-1,4-dihydro-4-oxoquinoline-3-carboxylic acid (ciprofloxacin) and its derivatives, as disclosed in PCT publications WO 2005/026165, WO 2005/049602, and WO 2005/033108, and European Patent publications EP 0343524, EP 0688772, EP 0153163, EP 0159174; as well as “water-solubilizing groups” described in United States patent publication 2006/0035891. The disclosure of each of these patent publications is incorporated herein by reference.

The compounds disclosed herein also include partially and fully deuterated variants. In certain embodiments, one or more deuterium atoms are present for kinetic studies. One of ordinary skill in the art can select the sites at which such deuterium atoms are present.

Also included in the present invention are salts, particularly pharmaceutically acceptable salts, of the sirtuin-modulating compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion (e.g., a halide such as bromide, chloride, or fluoride, particularly bromide).

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

According to another embodiment, the present invention provides methods of producing the above-defined sirtuin-modulating compounds. The compounds may be synthesized using conventional techniques. Advantageously, these compounds are conveniently synthesized from readily available starting materials.

Synthetic chemistry transformations and methodologies useful in synthesizing the sirtuin-modulating compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

In an exemplary embodiment, a sirtuin-modulating compound may traverse the cytoplasmic membrane of a cell. For example, a compound may have a cell-permeability of at least about 20%, 50%, 75%, 80%, 90% or 95%.

Sirtuin-modulating compounds described herein may also have one or more of the following characteristics: the compound may be essentially non-toxic to a cell or subject; the sirtuin-modulating compound may be an organic molecule or a small molecule of 2000 amu or less, 1000 amu or less; a compound may have a half-life under normal atmospheric conditions of at least about 30 days, 60 days, 120 days, 6 months or 1 year; the compound may have a half-life in solution of at least about 30 days, 60 days, 120 days, 6 months or 1 year; a sirtuin-modulating compound may be more stable in solution than resveratrol by at least a factor of about 50%, 2 fold, 5 fold, 10 fold, 30 fold, 50 fold or 100 fold; a sirtuin-modulating compound may promote deacetylation of the DNA repair factor Ku70; a sirtuin-modulating compound may promote deacetylation of RelA/p65; a compound may increase general turnover rates and enhance the sensitivity of cells to TNF-induced apoptosis.

In certain embodiments, a sirtuin-modulating compound does not have any substantial ability to inhibit a histone deacetylase (HDACs) class I, a HDAC class II, or HDACs I and II, at concentrations (e.g., in vivo) effective for modulating the deacetylase activity of the sirtuin. For instance, in preferred embodiments the sirtuin-modulating compound is a sirtuin-activating compound and is chosen to have an EC₅₀ for activating sirtuin deacetylase activity that is at least 5 fold less than the EC₅₀ for inhibition of an HDAC I and/or HDAC II, and even more preferably at least 10 fold, 100 fold or even 1000 fold less. Methods for assaying HDAC I and/or HDAC II activity are well known in the art and kits to perform such assays may be purchased commercially. See e.g., BioVision, Inc. (Mountain View, Calif.; world wide web at biovision.com) and Thomas Scientific (Swedesboro, N.J.; world wide web at tomassci.com).

In certain embodiments, a sirtuin-modulating compound does not have any substantial ability to modulate sirtuin homologs. In one embodiment, an activator of a human sirtuin protein may not have any substantial ability to activate a sirtuin protein from lower eukaryotes, particularly yeast or human pathogens, at concentrations (e.g., in vivo) effective for activating the deacetylase activity of human sirtuin. For example, a sirtuin-activating compound may be chosen to have an EC₅₀ for activating a human sirtuin, such as SIRT1 and/or SIRT3, deacetylase activity that is at least 5 fold less than the EC₅₀ for activating a yeast sirtuin, such as Sir2 (such as Candida, S. cerevisiae, etc.), and even more preferably at least 10 fold, 100 fold or even 1000 fold less. In another embodiment, an inhibitor of a sirtuin protein from lower eukaryotes, particularly yeast or human pathogens, does not have any substantial ability to inhibit a sirtuin protein from humans at concentrations (e.g., in vivo) effective for inhibiting the deacetylase activity of a sirtuin protein from a lower eukaryote. For example, a sirtuin-inhibiting compound may be chosen to have an IC₅₀ for inhibiting a human sirtuin, such as SIRT1 and/or SIRT3, deacetylase activity that is at least 5 fold less than the IC₅₀ for inhibiting a yeast sirtuin, such as Sir2 (such as Candida, S. cerevisiae, etc.), and even more preferably at least 10 fold, 100 fold or even 1000 fold less.

In certain embodiments, a sirtuin-modulating compound may have the ability to modulate one or more sirtuin protein homologs, such as, for example, one or more of human SIRT1, SIRT2, SIRT3, SIRT4, SIRT5, SIRT6, or SIRT7. In one embodiment, a sirtuin-modulating compound has the ability to modulate both a SIRT1 and a SIRT3 protein.

In other embodiments, a SIRT1 modulator does not have any substantial ability to modulate other sirtuin protein homologs, such as, for example, one or more of human SIRT2, SIRT3, SIRT4, SIRT5, SIRT6, or SIRT7, at concentrations (e.g., in vivo) effective for modulating the deacetylase activity of human SIRT1. For example, a sirtuin-modulating compound may be chosen to have an ED₅₀ for modulating human SIRT1 deacetylase activity that is at least 5 fold less than the ED₅₀ for modulating one or more of human SIRT2, SIRT3, SIRT4, SIRT5, SIRT6, or SIRT7, and even more preferably at least 10 fold, 100 fold or even 1000 fold less. In one embodiment, a SIRT1 modulator does not have any substantial ability to modulate a SIRT3 protein.

In other embodiments, a SIRT3 modulator does not have any substantial ability to modulate other sirtuin protein homologs, such as, for example, one or more of human SIRT1, SIRT2, SIRT4, SIRT5, SIRT6, or SIRT7, at concentrations (e.g., in vivo) effective for modulating the deacetylase activity of human SIRT3. For example, a sirtuin-modulating compound may be chosen to have an ED₅₀ for modulating human SIRT3 deacetylase activity that is at least 5 fold less than the ED₅₀ for modulating one or more of human SIRT1, SIRT2, SIRT4, SIRT5, SIRT6, or SIRT7, and even more preferably at least 10 fold, 100 fold or even 1000 fold less. In one embodiment, a SIRT3 modulator does not have any substantial ability to modulate a SIRT1 protein.

In certain embodiments, a sirtuin-modulating compound may have a binding affinity for a sirtuin protein of about 10⁻⁹M, 10⁻¹⁰ M, 10⁻¹¹M, 10⁻¹²M or less. A sirtuin-modulating compound may reduce (activator) or increase (inhibitor) the apparent Km of a sirtuin protein for its substrate or NAD+ (or other cofactor) by a factor of at least about 2, 3, 4, 5, 10, 20, 30, 50 or 100. In certain embodiments, Km values are determined using the mass spectrometry assay described herein. Preferred activating compounds reduce the Km of a sirtuin for its substrate or cofactor to a greater extent than caused by resveratrol at a similar concentration or reduce the Km of a sirtuin for its substrate or cofactor similar to that caused by resveratrol at a lower concentration. A sirtuin-modulating compound may increase the Vmax of a sirtuin protein by a factor of at least about 2, 3, 4, 5, 10, 20, 30, 50 or 100. A sirtuin-modulating compound may have an ED50 for modulating the deacetylase activity of a SIRT1 and/or SIRT3 protein of less than about 1 nM, less than about 10 nM, less than about 100 nM, less than about 1 μM, less than about 10 μM, less than about 100 μM, or from about 1-10 nM, from about 10-100 nM, from about 0.1-10 μM, from about 1-10 or from about 10-100 μM. A sirtuin-modulating compound may modulate the deacetylase activity of a SIRT1 and/or SIRT3 protein by a factor of at least about 5, 10, 20, 30, 50, or 100, as measured in a cellular assay or in a cell based assay. A sirtuin-activating compound may cause at least about 10%, 30%, 50%, 80%, 2 fold, 5 fold, 10 fold, 50 fold or 100 fold greater induction of the deacetylase activity of a sirtuin protein relative to the same concentration of resveratrol. A sirtuin-modulating compound may have an ED50 for modulating SIRT5 that is at least about 10 fold, 20 fold, 30 fold, 50 fold greater than that for modulating SIRT1 and/or SIRT3.

### 3. Exemplary Uses

In certain aspects, the invention provides methods for modulating the level and/or activity of a sirtuin protein and methods of use thereof.

In certain embodiments, the invention provides methods for using sirtuin-modulating compounds wherein the sirtuin-modulating compounds activate a sirtuin protein, e.g., increase the level and/or activity of a sirtuin protein. Sirtuin-modulating compounds that increase the level and/or activity of a sirtuin protein may be useful for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing, etc. The methods comprise administering to a subject in need thereof a pharmaceutically effective amount of a sirtuin-modulating compound, e.g., a sirtuin-activating compound.

While Applicants do not wish to be bound by theory, it is believed that activators of the instant invention may interact with a sirtuin at the same location within the sirtuin protein (e.g., active site or site affecting the Km or Vmax of the active site). It is believed that this is the reason why certain classes of sirtuin activators and inhibitors can have substantial structural similarity.

In certain embodiments, the sirtuin-modulating compounds described herein may be taken alone or in combination with other compounds. In one embodiment, a mixture of two or more sirtuin-modulating compounds may be administered to a subject in need thereof. In another embodiment, a sirtuin-modulating compound that increases the level and/or activity of a sirtuin protein may be administered with one or more of the following compounds: resveratrol, butein, fisetin, piceatannol, or quercetin. In an exemplary embodiment, a sirtuin-modulating compound that increases the level and/or activity of a sirtuin protein may be administered in combination with nicotinic acid. In another embodiment, a sirtuin-modulating compound that decreases the level and/or activity of a sirtuin protein may be administered with one or more of the following compounds: nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′,5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; gossypetin (3,5,7,8,3′,4′-hexahydroxyflavone), sirtinol; and splitomicin. In yet another embodiment, one or more sirtuin-modulating compounds may be administered with one or more therapeutic agents for the treatment or prevention of various diseases, including, for example, cancer, diabetes, neurodegenerative diseases, cardiovascular disease, blood clotting, inflammation, flushing, obesity, ageing, stress, etc. In various embodiments, combination therapies comprising a sirtuin-modulating compound may refer to (1) pharmaceutical compositions that comprise one or more sirtuin-modulating compounds in combination with one or more therapeutic agents (e.g., one or more therapeutic agents described herein); and (2) co-administration of one or more sirtuin-modulating compounds with one or more therapeutic agents wherein the sirtuin-modulating compound and therapeutic agent have not been formulated in the same compositions (but may be present within the same kit or package, such as a blister pack or other multi-chamber package; connected, separately sealed containers (e.g., foil pouches) that can be separated by the user; or a kit where the sirtuin modulating compound(s) and other therapeutic agent(s) are in separate vessels). When using separate formulations, the sirtuin-modulating compound may be administered at the same, intermittent, staggered, prior to, subsequent to, or combinations thereof, with the administration of another therapeutic agent.

In certain embodiments, methods for reducing, preventing or treating diseases or disorders using a sirtuin-modulating compound may also comprise increasing the protein level of a sirtuin, such as human SIRT1, SIRT2 and/or SIRT3, or homologs thereof. Increasing protein levels can be achieved by introducing into a cell one or more copies of a nucleic acid that encodes a sirtuin. For example, the level of a sirtuin can be increased in a mammalian cell by introducing into the mammalian cell a nucleic acid encoding the sirtuin, e.g., increasing the level of SIRT1 by introducing a nucleic acid encoding the amino acid sequence set forth in GenBank Accession No. NP_{—}036370 and/or increasing the level of SIRT3 by introducing a nucleic acid encoding the amino acid sequence set forth in GenBank Accession No. AAH01042.

A nucleic acid that is introduced into a cell to increase the protein level of a sirtuin may encode a protein that is at least about 80%, 85%, 90%, 95%, 98%, or 99% identical to the sequence of a sirtuin, e.g., SIRT1 and/or SIRT3 protein. For example, the nucleic acid encoding the protein may be at least about 80%, 85%, 90%, 95%, 98%, or 99% identical to a nucleic acid encoding a SIRT1 (e.g. GenBank Accession No. NM_{—}012238) and/or SIRT3 (e.g., GenBank Accession No. BC001042) protein. The nucleic acid may also be a nucleic acid that hybridizes, preferably under stringent hybridization conditions, to a nucleic acid encoding a wild-type sirtuin, e.g., SIRT1 and/or SIRT3 protein. Stringent hybridization conditions may include hybridization and a wash in 0.2×SSC at 65° C. When using a nucleic acid that encodes a protein that is different from a wild-type sirtuin protein, such as a protein that is a fragment of a wild-type sirtuin, the protein is preferably biologically active, e.g., is capable of deacetylation. It is only necessary to express in a cell a portion of the sirtuin that is biologically active. For example, a protein that differs from wild-type SIRT1 having GenBank Accession No. NP_{—}036370, preferably contains the core structure thereof. The core structure sometimes refers to amino acids 62-293 of GenBank Accession No. NP_{—}036370, which are encoded by nucleotides 237 to 932 of GenBank Accession No. NM_{—}012238, which encompasses the NAD binding as well as the substrate binding domains. The core domain of SIRT1 may also refer to about amino acids 261 to 447 of GenBank Accession No. NP_{—}036370, which are encoded by nucleotides 834 to 1394 of GenBank Accession No. NM_{—}012238; to about amino acids 242 to 493 of GenBank Accession No. NP_{—}036370, which are encoded by nucleotides 777 to 1532 of GenBank Accession No. NM_{—}012238; or to about amino acids 254 to 495 of GenBank Accession No. NP_{—}036370, which are encoded by nucleotides 813 to 1538 of GenBank Accession No. NM_{—}012238. Whether a protein retains a biological function, e.g., deacetylation capabilities, can be determined according to methods known in the art.

In certain embodiments, methods for reducing, preventing or treating diseases or disorders using a sirtuin-modulating compound may also comprise decreasing the protein level of a sirtuin, such as human SIRT1, SIRT2 and/or SIRT3, or homologs thereof. Decreasing a sirtuin protein level can be achieved according to methods known in the art. For example, an siRNA, an antisense nucleic acid, or a ribozyme targeted to the sirtuin can be expressed in the cell. A dominant negative sirtuin mutant, e.g., a mutant that is not capable of deacetylating, may also be used. For example, mutant H363Y of SIRT1, described, e.g., in Luo et al. (2001) Cell 107:137 can be used. Alternatively, agents that inhibit transcription can be used.

Methods for modulating sirtuin protein levels also include methods for modulating the transcription of genes encoding sirtuins, methods for stabilizing/destabilizing the corresponding mRNAs, and other methods known in the art.

### Aging/Stress

In one embodiment, the invention provides a method extending the lifespan of a cell, extending the proliferative capacity of a cell, slowing aging of a cell, promoting the survival of a cell, delaying cellular senescence in a cell, mimicking the effects of calorie restriction, increasing the resistance of a cell to stress, or preventing apoptosis of a cell, by contacting the cell with a sirtuin-modulating compound of the invention that increases the level and/or activity of a sirtuin protein. In an exemplary embodiment, the methods comprise contacting the cell with a sirtuin-activating compound.

The methods described herein may be used to increase the amount of time that cells, particularly primary cells (i.e., cells obtained from an organism, e.g., a human), may be kept alive in a cell culture. Embryonic stem (ES) cells and pluripotent cells, and cells differentiated therefrom, may also be treated with a sirtuin-modulating compound that increases the level and/or activity of a sirtuin protein to keep the cells, or progeny thereof, in culture for longer periods of time. Such cells can also be used for transplantation into a subject, e.g., after ex vivo modification.

In one embodiment, cells that are intended to be preserved for long periods of time may be treated with a sirtuin-modulating compound that increases the level and/or activity of a sirtuin protein. The cells may be in suspension (e.g., blood cells, serum, biological growth media, etc.) or in tissues or organs. For example, blood collected from an individual for purposes of transfusion may be treated with a sirtuin-modulating compound that increases the level and/or activity of a sirtuin protein to preserve the blood cells for longer periods of time. Additionally, blood to be used for forensic purposes may also be preserved using a sirtuin-modulating compound that increases the level and/or activity of a sirtuin protein. Other cells that may be treated to extend their lifespan or protect against apoptosis include cells for consumption, e.g., cells from non-human mammals (such as meat) or plant cells (such as vegetables).

Sirtuin-modulating compounds that increase the level and/or activity of a sirtuin protein may also be applied during developmental and growth phases in mammals, plants, insects or microorganisms, in order to, e.g., alter, retard or accelerate the developmental and/or growth process.

In another embodiment, sirtuin-modulating compounds that increase the level and/or activity of a sirtuin protein may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated with the sirtuin-modulating compound prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with a sirtuin-modulating compound or may have a subset of cells/tissue treated locally with a sirtuin-modulating compound that increases the level and/or activity of a sirtuin protein. In certain embodiments, the cells or tissue (or donor/recipient individuals) may additionally be treated with another therapeutic agent useful for prolonging graft survival, such as, for example, an immunosuppressive agent, a cytokine, an angiogenic factor, etc.

In yet other embodiments, cells may be treated with a sirtuin-modulating compound that increases the level and/or activity of a sirtuin protein in vivo, e.g., to increase their lifespan or prevent apoptosis. For example, skin can be protected from aging (e.g., developing wrinkles, loss of elasticity, etc.) by treating skin or epithelial cells with a sirtuin-modulating compound that increases the level and/or activity of a sirtuin protein. In an exemplary embodiment, skin is contacted with a pharmaceutical or cosmetic composition comprising a sirtuin-modulating compound that increases the level and/or activity of a sirtuin protein. Exemplary skin afflictions or skin conditions that may be treated in accordance with the methods described herein include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, psoriasis, skin cancer and the effects of natural aging. In another embodiment, sirtuin-modulating compounds that increase the level and/or activity of a sirtuin protein may be used for the treatment of wounds and/or burns to promote healing, including, for example, first-, second- or third-degree burns and/or a thermal, chemical or electrical burns. The formulations may be administered topically, to the skin or mucosal tissue.

Topical formulations comprising one or more sirtuin-modulating compounds that increase the level and/or activity of a sirtuin protein may also be used as preventive, e.g., chemopreventive, compositions. When used in a chemopreventive method, susceptible skin is treated prior to any visible condition in a particular individual.

Sirtuin-modulating compounds may be delivered locally or systemically to a subject. In one embodiment, a sirtuin-modulating compound is delivered locally to a tissue or organ of a subject by injection, topical formulation, etc.

In another embodiment, a sirtuin-modulating compound that increases the level and/or activity of a sirtuin protein may be used for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the method does not act by decreasing the rate of occurrence of diseases that shorten the lifespan of a subject. In certain embodiments, a method does not act by reducing the lethality caused by a disease, such as cancer.

In yet another embodiment, a sirtuin-modulating compound that increases the level and/or activity of a sirtuin protein may be administered to a subject in order to generally increase the lifespan of its cells and to protect its cells against stress and/or against apoptosis. It is believed that treating a subject with a compound described herein is similar to subjecting the subject to hormesis, i.e., mild stress that is beneficial to organisms and may extend their lifespan.

Sirtuin-modulating compounds that increase the level and/or activity of a sirtuin protein may be administered to a subject to prevent aging and aging-related consequences or diseases, such as stroke, heart disease, heart failure, arthritis, high blood pressure, and Alzheimer's disease. Other conditions that can be treated include ocular disorders, e.g., associated with the aging of the eye, such as cataracts, glaucoma, and macular degeneration. Sirtuin-modulating compounds that increase the level and/or activity of a sirtuin protein can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure or radiation exposure.

Sirtuin-modulating compounds that increase the level and/or activity of a sirtuin protein can also be administered to a subject suffering from an acute disease, e.g., damage to an organ or tissue, e.g., a subject suffering from stroke or myocardial infarction or a subject suffering from a spinal cord injury. Sirtuin-modulating compounds that increase the level and/or activity of a sirtuin protein may also be used to repair an alcoholic's liver.

### Cardiovascular Disease

In another embodiment, the invention provides a method for treating and/or preventing a cardiovascular disease by administering to a subject in need thereof a sirtuin-modulating compound that increases the level and/or activity of a sirtuin protein.

Cardiovascular diseases that can be treated or prevented using the sirtuin-modulating compounds that increase the level and/or activity of a sirtuin protein include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using compounds and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The sirtuin-modulating compounds that increase the level and/or activity of a sirtuin protein may also be used for increasing HDL levels in plasma of an individual.

Yet other disorders that may be treated with sirtuin-modulating compounds that increase the level and/or activity of a sirtuin protein include restenosis, e.g., following coronary intervention, and disorders relating to an abnormal level of high density and low density cholesterol.

In one embodiment, a sirtuin-modulating compound that increases the level and/or activity of a sirtuin protein may be administered as part of a combination therapeutic with another cardiovascular agent. In one embodiment, a sirtuin-modulating compound that increases the level and/or activity of a sirtuin protein may be administered as part of a combination therapeutic with an anti-arrhythmia agent. In another embodiment, a sirtuin-modulating compound that increases the level and/or activity of a sirtuin protein may be administered as part of a combination therapeutic with another cardiovascular agent.

### Cell Death/Cancer

Sirtuin-modulating compounds that increase the level and/or activity of a sirtuin protein may be administered to subjects who have recently received or are likely to receive a dose of radiation or toxin. In one embodiment, the dose of radiation or toxin is received as part of a work-related or medical procedure, e.g., administered as a prophylactic measure. In another embodiment, the radiation or toxin exposure is received unintentionally. In such a case, the compound is preferably administered as soon as possible after the exposure to inhibit apoptosis and the subsequent development of acute radiation syndrome.

Sirtuin-modulating compounds may also be used for treating and/or preventing cancer. In certain embodiments, sirtuin-modulating compounds that increase the level and/or activity of a sirtuin protein may be used for treating and/or preventing cancer. Calorie restriction has been linked to a reduction in the incidence of age-related disorders including cancer. Accordingly, an increase in the level and/or activity of a sirtuin protein may be useful for treating and/or preventing the incidence of age-related disorders, such as, for example, cancer. Exemplary cancers that may be treated using a sirtuin-modulating compound are those of the brain and kidney; hormone-dependent cancers including breast, prostate, testicular, and ovarian cancers; lymphomas, and leukemias. In cancers associated with solid tumors, a modulating compound may be administered directly into the tumor. Cancer of blood cells, e.g., leukemia, can be treated by administering a modulating compound into the blood stream or into the bone marrow. Benign cell growth, e.g., warts, can also be treated. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-1 associated malignant and benign disorders can also be treated by administration of sirtuin-modulating compound. Alternatively, cells can be obtained from a subject, treated ex vivo to remove certain undesirable cells, e.g., cancer cells, and administered back to the same or a different subject.

Chemotherapeutic agents may be co-administered with modulating compounds described herein as having anti-cancer activity, e.g., compounds that induce apoptosis, compounds that reduce lifespan or compounds that render cells sensitive to stress. Chemotherapeutic agents may be used by themselves with a sirtuin-modulating compound described herein as inducing cell death or reducing lifespan or increasing sensitivity to stress and/or in combination with other chemotherapeutics agents. In addition to conventional chemotherapeutics, the sirtuin-modulating compounds described herein may also be used with antisense RNA, RNAi or other polynucleotides to inhibit the expression of the cellular components that contribute to unwanted cellular proliferation.

Combination therapies comprising sirtuin-modulating compounds and a conventional chemotherapeutic agent may be advantageous over combination therapies known in the art because the combination allows the conventional chemotherapeutic agent to exert greater effect at lower dosage. In a preferred embodiment, the effective dose (ED₅₀) for a chemotherapeutic agent, or combination of conventional chemotherapeutic agents, when used in combination with a sirtuin-modulating compound is at least 2 fold less than the ED₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a sirtuin-modulating compound described herein can be at least 2 fold greater than the TI for conventional chemotherapeutic regimen alone, and even more preferably at 5 fold, 10 fold or even 25 fold greater.

### Neuronal Diseases/Disorders

In certain aspects, sirtuin-modulating compounds that increase the level and/or activity of a sirtuin protein can be used to treat patients suffering from neurodegenerative diseases, and traumatic or mechanical injury to the central nervous system (CNS), spinal cord or peripheral nervous system (PNS). Neurodegenerative disease typically involves reductions in the mass and volume of the human brain, which may be due to the atrophy and/or death of brain cells, which are far more profound than those in a healthy person that are attributable to aging. Neurodegenerative diseases can evolve gradually, after a long period of normal brain function, due to progressive degeneration (e.g., nerve cell dysfunction and death) of specific brain regions. Alternatively, neurodegenerative diseases can have a quick onset, such as those associated with trauma or toxins. The actual onset of brain degeneration may precede clinical expression by many years. Examples of neurodegenerative diseases include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia. Sirtuin-modulating compounds that increase the level and/or activity of a sirtuin protein can be used to treat these disorders and others as described below.

AD is a CNS disorder that results in memory loss, unusual behavior, personality changes, and a decline in thinking abilities. These losses are related to the death of specific types of brain cells and the breakdown of connections and their supporting network (e.g. glial cells) between them. The earliest symptoms include loss of recent memory, faulty judgment, and changes in personality. PD is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles.

HD is another neurodegenerative disease that causes uncontrolled movements, loss of intellectual faculties, and emotional disturbance. Tay-Sachs disease and Sandhoff disease are glycolipid storage diseases where GM2 ganglioside and related glycolipidssubstrates for β-hexosaminidase accumulate in the nervous system and trigger acute neurodegeneration.

It is well-known that apoptosis plays a role in AIDS pathogenesis in the immune system. However, HIV-1 also induces neurological disease, which can be treated with sirtuin-modulating compounds of the invention.

Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats. Sirtuin-modulating compounds that increase the level and/or activity of a sirtuin protein may be useful for treating or preventing neuronal loss due to these prior diseases.

In another embodiment, a sirtuin-modulating compound that increases the level and/or activity of a sirtuin protein may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. Those with distal axonopathies usually present with symmetrical glove-stocking sensori-motor disturbances. Deep tendon reflexes and autonomic nervous system (ANS) functions are also lost or diminished in affected areas.

Diabetic neuropathies are neuropathic disorders that are associated with diabetes mellitus. Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy.

Peripheral neuropathy is the medical term for damage to nerves of the peripheral nervous system, which may be caused either by diseases of the nerve or from the side-effects of systemic illness. Major causes of peripheral neuropathy include seizures, nutritional deficiencies, and HIV, though diabetes is the most likely cause.

In an exemplary embodiment, a sirtuin-modulating compound that increases the level and/or activity of a sirtuin protein may be used to treat or prevent multiple sclerosis (MS), including relapsing MS and monosymptomatic MS, and other demyelinating conditions, such as, for example, chromic inflammatory demyelinating polyneuropathy (CIDP), or symptoms associated therewith.

In yet another embodiment, a sirtuin-modulating compound that increases the level and/or activity of a sirtuin protein may be used to treat trauma to the nerves, including, trauma due to disease, injury (including surgical intervention), or environmental trauma (e.g., neurotoxins, alcoholism, etc.).

Sirtuin-modulating compounds that increase the level and/or activity of a sirtuin protein may also be useful to prevent, treat, and alleviate symptoms of various PNS disorders. The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used.

PNS diseases treatable with sirtuin-modulating compounds that increase the level and/or activity of a sirtuin protein include: diabetes, leprosy, Charcot-Marie-Tooth disease, Guillain-Barré syndrome and Brachial Plexus Neuropathies (diseases of the cervical and first thoracic roots, nerve trunks, cords, and peripheral nerve components of the brachial plexus.

In another embodiment, a sirtuin activating compound may be used to treat or prevent a polyglutamine disease. Exemplary polyglutamine diseases include Spinobulbar muscular atrophy (Kennedy disease), Huntington's Disease (HD), Dentatorubral-pallidoluysian atrophy (Haw River syndrome), Spinocerebellar ataxia type 1, Spinocerebellar ataxia type 2, Spinocerebellar ataxia type 3 (Machado-Joseph disease), Spinocerebellar ataxia type 6, Spinocerebellar ataxia type 7, and Spinocerebellar ataxia type 17.

In certain embodiments, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell.

Another aspect encompasses administrating a sirtuin activating compound to a subject to treat a central nervous system ischemic condition. A number of central nervous system ischemic conditions may be treated by the sirtuin activating compounds described herein. In one embodiment, the ischemic condition is a stroke that results in any type of ischemic central nervous system damage, such as apoptotic or necrotic cell death, cytoxic edema or central nervous system tissue anoxia. The stroke may impact any area of the brain or be caused by any etiology commonly known to result in the occurrence of a stroke. In one alternative of this embodiment, the stroke is a brain stem stroke. In another alternative of this embodiment, the stroke is a cerebellar stroke. In still another embodiment, the stroke is an embolic stroke. In yet another alternative, the stroke may be a hemorrhagic stroke. In a further embodiment, the stroke is a thrombotic stroke.

In yet another aspect, a sirtuin activating compound may be administered to reduce infarct size of the ischemic core following a central nervous system ischemic condition. Moreover, a sirtuin activating compound may also be beneficially administered to reduce the size of the ischemic penumbra or transitional zone following a central nervous system ischemic condition.

In one embodiment, a combination drug regimen may include drugs or compounds for the treatment or prevention of neurodegenerative disorders or secondary conditions associated with these conditions. Thus, a combination drug regimen may include one or more sirtuin activators and one or more anti-neurodegeneration agents.

### Blood Coagulation Disorders

In other aspects, sirtuin-modulating compounds that increase the level and/or activity of a sirtuin protein can be used to treat or prevent blood coagulation disorders (or hemostatic disorders). As used interchangeably herein, the terms “hemostasis”, “blood coagulation,” and “blood clotting” refer to the control of bleeding, including the physiological properties of vasoconstriction and coagulation. Blood coagulation assists in maintaining the integrity of mammalian circulation after injury, inflammation, disease, congenital defect, dysfunction or other disruption. Further, the formation of blood clots does not only limit bleeding in case of an injury (hemostasis), but may lead to serious organ damage and death in the context of atherosclerotic diseases by occlusion of an important artery or vein. Thrombosis is thus blood clot formation at the wrong time and place.

Accordingly, the present invention provides anticoagulation and antithrombotic treatments aiming at inhibiting the formation of blood clots in order to prevent or treat blood coagulation disorders, such as myocardial infarction, stroke, loss of a limb by peripheral artery disease or pulmonary embolism.

As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis.

In one aspect, the invention provides a method for reducing or inhibiting hemostasis in a subject by administering a sirtuin-modulating compound that increases the level and/or activity of a sirtuin protein. The compositions and methods disclosed herein are useful for the treatment or prevention of thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites.

In another embodiment, a combination drug regimen may include drugs or compounds for the treatment or prevention of blood coagulation disorders or secondary conditions associated with these conditions. Thus, a combination drug regimen may include one or more sirtuin-modulating compounds that increase the level and/or activity of a sirtuin protein and one or more anti-coagulation or anti-thrombosis agents.

### Weight Control

In another aspect, sirtuin-modulating compounds that increase the level and/or activity of a sirtuin protein may be used for treating or preventing weight gain or obesity in a subject. For example, sirtuin-modulating compounds that increase the level and/or activity of a sirtuin protein may be used, for example, to treat or prevent hereditary obesity, dietary obesity, hormone related obesity, obesity related to the administration of medication, to reduce the weight of a subject, or to reduce or prevent weight gain in a subject. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof.

In yet other embodiments, sirtuin-modulating compounds that increase the level and/or activity of a sirtuin protein may be administered to subjects suffering from a variety of other diseases and conditions that may be treated or prevented by promoting weight loss in the subject. Such diseases include, for example, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self esteem). Finally, patients with AIDS can develop lipodystrophy or insulin resistance in response to combination therapies for AIDS.

In another embodiment, sirtuin-modulating compounds that increase the level and/or activity of a sirtuin protein may be used for inhibiting adipogenesis or fat cell differentiation, whether in vitro or in vivo. Such methods may be used for treating or preventing obesity.

In other embodiments, sirtuin-modulating compounds that increase the level and/or activity of a sirtuin protein may be used for reducing appetite and/or increasing satiety, thereby causing weight loss or avoidance of weight gain. A subject in need of such a treatment may be a subject who is overweight, obese or a subject likely to become overweight or obese. The method may comprise administering daily or, every other day, or once a week, a dose, e.g., in the form of a pill, to a subject. The dose may be an “appetite reducing dose.”

In an exemplary embodiment, sirtuin-modulating compounds that increase the level and/or activity of a sirtuin protein may be administered as a combination therapy for treating or preventing weight gain or obesity. For example, one or more sirtuin-modulating compounds that increase the level and/or activity of a sirtuin protein may be administered in combination with one or more anti-obesity agents.

In another embodiment, sirtuin-modulating compounds that increase the level and/or activity of a sirtuin protein may be administered to reduce drug-induced weight gain. For example, a sirtuin-modulating compound that increases the level and/or activity of a sirtuin protein may be administered as a combination therapy with medications that may stimulate appetite or cause weight gain, in particular, weight gain due to factors other than water retention.

### Metabolic Disorders/Diabetes

In another aspect, sirtuin-modulating compounds that increase the level and/or activity of a sirtuin protein may be used for treating or preventing a metabolic disorder, such as insulin-resistance, a pre-diabetic state, type II diabetes, and/or complications thereof. Administration of a sirtuin-modulating compounds that increases the level and/or activity of a sirtuin protein may increase insulin sensitivity and/or decrease insulin levels in a subject. A subject in need of such a treatment may be a subject who has insulin resistance or other precursor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

In an exemplary embodiment, sirtuin-modulating compounds that increase the level and/or activity of a sirtuin protein may be administered as a combination therapy for treating or preventing a metabolic disorder. For example, one or more sirtuin-modulating compounds that increase the level and/or activity of a sirtuin protein may be administered in combination with one or more anti-diabetic agents.

### Inflammatory Diseases

In other aspects, sirtuin-modulating compounds that increase the level and/or activity of a sirtuin protein can be used to treat or prevent a disease or disorder associated with inflammation. Sirtuin-modulating compounds that increase the level and/or activity of a sirtuin protein may be administered prior to the onset of, at, or after the initiation of inflammation. When used prophylactically, the compounds are preferably provided in advance of any inflammatory response or symptom. Administration of the compounds may prevent or attenuate inflammatory responses or symptoms.

In another embodiment, sirtuin-modulating compounds that increase the level and/or activity of a sirtuin protein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C.

Additionally, sirtuin-modulating compounds that increase the level and/or activity of a sirtuin protein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain embodiments, one or more sirtuin-modulating compounds that increase the level and/or activity of a sirtuin protein may be taken alone or in combination with other compounds useful for treating or preventing inflammation.

### Flushing

In another aspect, sirtuin-modulating compounds that increase the level and/or activity of a sirtuin protein may be used for reducing the incidence or severity of flushing and/or hot flashes which are symptoms of a disorder. For instance, the subject method includes the use of sirtuin-modulating compounds that increase the level and/or activity of a sirtuin protein, alone or in combination with other agents, for reducing incidence or severity of flushing and/or hot flashes in cancer patients. In other embodiments, the method provides for the use of sirtuin-modulating compounds that increase the level and/or activity of a sirtuin protein to reduce the incidence or severity of flushing and/or hot flashes in menopausal and post-menopausal woman.

In another aspect, sirtuin-modulating compounds that increase the level and/or activity of a sirtuin protein may be used as a therapy for reducing the incidence or severity of flushing and/or hot flashes which are side-effects of another drug therapy, e.g., drug-induced flushing. In certain embodiments, a method for treating and/or preventing drug-induced flushing comprises administering to a patient in need thereof a formulation comprising at least one flushing inducing compound and at least one sirtuin-modulating compound that increases the level and/or activity of a sirtuin protein. In other embodiments, a method for treating drug induced flushing comprises separately administering one or more compounds that induce flushing and one or more sirtuin-modulating compounds, e.g., wherein the sirtuin-modulating compound and flushing inducing agent have not been formulated in the same compositions. When using separate formulations, the sirtuin-modulating compound may be administered (1) at the same as administration of the flushing inducing agent, (2) intermittently with the flushing inducing agent, (3) staggered relative to administration of the flushing inducing agent, (4) prior to administration of the flushing inducing agent, (5) subsequent to administration of the flushing inducing agent, and (6) various combination thereof. Exemplary flushing inducing agents include, for example, niacin, faloxifene, antidepressants, anti-psychotics, chemotherapeutics, calcium channel blockers, and antibiotics.

In one embodiment, sirtuin-modulating compounds that increase the level and/or activity of a sirtuin protein may be used to reduce flushing side effects of a vasodilator or an antilipemic agent (including anticholesteremic agents and lipotropic agents). In an exemplary embodiment, a sirtuin-modulating compound that increases the level and/or activity of a sirtuin protein may be used to reduce flushing associated with the administration of niacin.

In another embodiment, the invention provides a method for treating and/or preventing hyperlipidemia with reduced flushing side effects. In another representative embodiment, the method involves the use of sirtuin-modulating compounds that increase the level and/or activity of a sirtuin protein to reduce flushing side effects of raloxifene. In another representative embodiment, the method involves the use of sirtuin-modulating compounds that increase the level and/or activity of a sirtuin protein to reduce flushing side effects of antidepressants or anti-psychotic agent. For instance, sirtuin-modulating compounds that increase the level and/or activity of a sirtuin protein can be used in conjunction (administered separately or together) with a serotonin reuptake inhibitor, or a 5HT2 receptor antagonist.

In certain embodiments, sirtuin-modulating compounds that increase the level and/or activity of a sirtuin protein may be used as part of a treatment with a serotonin reuptake inhibitor (SR1) to reduce flushing. In still another representative embodiment, sirtuin-modulating compounds that increase the level and/or activity of a sirtuin protein may be used to reduce flushing side effects of chemotherapeutic agents, such as cyclophosphamide and tamoxifen.

In another embodiment, sirtuin-modulating compounds that increase the level and/or activity of a sirtuin protein may be used to reduce flushing side effects of calcium channel blockers, such as amlodipine.

In another embodiment, sirtuin-modulating compounds that increase the level and/or activity of a sirtuin protein may be used to reduce flushing side effects of antibiotics. For example, sirtuin-modulating compounds that increase the level and/or activity of a sirtuin protein can be used in combination with levofloxacin.

### Ocular Disorders

One aspect of the present invention is a method for inhibiting, reducing or otherwise treating vision impairment by administering to a patient a therapeutic dosage of sirtuin modulator selected from a compound disclosed herein, or a pharmaceutically acceptable salt, prodrug or a metabolic derivative thereof.

In certain aspects of the invention, the vision impairment is caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response such as in optic neuritis.

In certain aspects of the invention, the vision impairment is caused by retinal damage. In particular embodiments, retinal damage is caused by disturbances in blood flow to the eye (e.g., arteriosclerosis, vasculitis). In particular embodiments, retinal damage is caused by disrupton of the macula (e.g., exudative or non-exudative macular degeneration).

Exemplary retinal diseases include Exudative Age Related Macular Degeneration, Nonexudative Age Related Macular Degeneration, Retinal Electronic Prosthesis and RPE Transplantation Age Related Macular Degeneration, Acute Multifocal Placoid Pigment Epitheliopathy, Acute Retinal Necrosis, Best Disease, Branch Retinal Artery Occlusion, Branch Retinal Vein Occlusion, Cancer Associated and Related Autoimmune Retinopathies, Central Retinal Artery Occlusion, Central Retinal Vein Occlusion, Central Serous Chorioretinopathy, Eales Disease, Epimacular Membrane, Lattice Degeneration, Macroaneurysm, Diabetic Macular Edema, Irvine-Gass Macular Edema, Macular Hole, Subretinal Neovascular Membranes, Diffuse Unilateral Subacute Neuroretinitis, Nonpseudophakic Cystoid Macular Edema, Presumed Ocular Histoplasmosis Syndrome, Exudative Retinal Detachment, Postoperative Retinal Detachment, Proliferative Retinal Detachment, Rhegmatogenous Retinal Detachment, Tractional Retinal Detachment, Retinitis Pigmentosa, CMV Retinitis, Retinoblastoma, Retinopathy of Prematurity, Birdshot Retinopathy, Background Diabetic Retinopathy, Proliferative Diabetic Retinopathy, Hemoglobinopathies Retinopathy, Purtscher Retinopathy, Valsalva Retinopathy, Juvenile Retinoschisis, Senile Retinoschisis, Terson Syndrome and White Dot Syndromes.

Other exemplary diseases include ocular bacterial infections (e.g. conjunctivitis, keratitis, tuberculosis, syphilis, gonorrhea), viral infections (e.g. Ocular Herpes Simplex Virus, Varicella Zoster Virus, Cytomegalovirus retinitis, Human Immunodeficiency Virus (HIV)) as well as progressive outer retinal necrosis secondary to HIV or other HIV-associated and other immunodeficiency-associated ocular diseases. In addition, ocular diseases include fungal infections (e.g. Candida choroiditis, histoplasmosis), protozoal infections (e.g. toxoplasmosis) and others such as ocular toxocariasis and sarcoidosis.

One aspect of the invention is a method for inhibiting, reducing or treating vision impairment in a subject undergoing treatment with a chemotherapeutic drug (e.g., a neurotoxic drug, a drug that raises intraocular pressure such as a steroid), by administering to the subject in need of such treatment a therapeutic dosage of a sirtuin modulator disclosed herein.

Another aspect of the invention is a method for inhibiting, reducing or treating vision impairment in a subject undergoing surgery, including ocular or other surgeries performed in the prone position such as spinal cord surgery, by administering to the subject in need of such treatment a therapeutic dosage of a sirtuin modulator disclosed herein. Ocular surgeries include cataract, iridotomy and lens replacements.

Another aspect of the invention is the treatment, including inhibition and prophylactic treatment, of age related ocular diseases include cataracts, dry eye, age-related macular degeneration (AMD), retinal damage and the like, by administering to the subject in need of such treatment a therapeutic dosage of a sirtuin modulator disclosed herein.

Another aspect of the invention is the prevention or treatment of damage to the eye caused by stress, chemical insult or radiation, by administering to the subject in need of such treatment a therapeutic dosage of a sirtuin modulator disclosed herein. Radiation or electromagnetic damage to the eye can include that caused by CRT's or exposure to sunlight or UV.

In one embodiment, a combination drug regimen may include drugs or compounds for the treatment or prevention of ocular disorders or secondary conditions associated with these conditions. Thus, a combination drug regimen may include one or more sirtuin activators and one or more therapeutic agents for the treatment of an ocular disorder.

In one embodiment, a sirtuin modulator can be administered in conjunction with a therapy for reducing intraocular pressure. In another embodiment, a sirtuin modulator can be administered in conjunction with a therapy for treating and/or preventing glaucoma. In yet another embodiment, a sirtuin modulator can be administered in conjunction with a therapy for treating and/or preventing optic neuritis. In one embodiment, a sirtuin modulator can be administered in conjunction with a therapy for treating and/or preventing CMV Retinopathy. In another embodiment, a sirtuin modulator can be administered in conjunction with a therapy for treating and/or preventing multiple sclerosis.

### Mitochondrial-Associated Diseases and Disorders

In certain embodiments, the invention provides methods for treating diseases or disorders that would benefit from increased mitochondrial activity. The methods involve administering to a subject in need thereof a therapeutically effective amount of a sirtuin activating compound. Increased mitochondrial activity refers to increasing activity of the mitochondria while maintaining the overall numbers of mitochondria (e.g., mitochondrial mass), increasing the numbers of mitochondria thereby increasing mitochondrial activity (e.g., by stimulating mitochondrial biogenesis), or combinations thereof. In certain embodiments, diseases and disorders that would benefit from increased mitochondrial activity include diseases or disorders associated with mitochondrial dysfunction.

In certain embodiments, methods for treating diseases or disorders that would benefit from increased mitochondrial activity may comprise identifying a subject suffering from a mitochondrial dysfunction. Methods for diagnosing a mitochondrial dysfunction may involve molecular genetic, pathologic and/or biochemical analyses. Diseases and disorders associated with mitochondrial dysfunction include diseases and disorders in which deficits in mitochondrial respiratory chain activity contribute to the development of pathophysiology of such diseases or disorders in a mammal. Diseases or disorders that would benefit from increased mitochondrial activity generally include for example, diseases in which free radical mediated oxidative injury leads to tissue degeneration, diseases in which cells inappropriately undergo apoptosis, and diseases in which cells fail to undergo apoptosis.

In certain embodiments, the invention provides methods for treating a disease or disorder that would benefit from increased mitochondrial activity that involves administering to a subject in need thereof one or more sirtuin activating compounds in combination with another therapeutic agent such as, for example, an agent useful for treating mitochondrial dysfunction or an agent useful for reducing a symptom associated with a disease or disorder involving mitochondrial dysfunction.

In exemplary embodiments, the invention provides methods for treating diseases or disorders that would benefit from increased mitochondrial activity by administering to a subject a therapeutically effective amount of a sirtuin activating compound. Exemplary diseases or disorders include, for example, neuromuscular disorders (e.g., Friedreich's Ataxia, muscular dystrophy, multiple sclerosis, etc.), disorders of neuronal instability (e.g., seizure disorders, migrane, etc.), developmental delay, neurodegenerative disorders (e.g., Alzheimer's Disease, Parkinson's Disease, amyotrophic lateral sclerosis, etc.), ischemia, renal tubular acidosis, age-related neurodegeneration and cognitive decline, chemotherapy fatigue, age-related or chemotherapy-induced menopause or irregularities of menstrual cycling or ovulation, mitochondrial myopathies, mitochondrial damage (e.g., calcium accumulation, excitotoxicity, nitric oxide exposure, hypoxia, etc.), and mitochondrial deregulation.

Muscular dystrophy refers to a family of diseases involving deterioration of neuromuscular structure and function, often resulting in atrophy of skeletal muscle and myocardial dysfunction, such as Duchenne muscular dystrophy. In certain embodiments, sirtuin activating compounds may be used for reducing the rate of decline in muscular functional capacities and for improving muscular functional status in patients with muscular dystrophy.

In certain embodiments, sirtuin modulating compounds may be useful for treatment mitochondrial myopathies. Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external opthalmoplegia, the Kearns-Sayre syndrome (with opthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal).

In certain embodiments, sirtuin activating compounds may be useful for treating patients suffering from toxic damage to mitochondria, such as, toxic damage due to calcium accumulation, excitotoxicity, nitric oxide exposure, drug induced toxic damage, or hypoxia.

In certain embodiments, sirtuin activating compounds may be useful for treating diseases or disorders associated with mitochondrial deregulation.

### Muscle Performance

In other embodiments, the invention provides methods for enhancing muscle performance by administering a therapeutically effective amount of a sirtuin activating compound. For example, sirtuin activating compounds may be useful for improving physical endurance (e.g., ability to perform a physical task such as exercise, physical labor, sports activities, etc.), inhibiting or retarding physical fatigues, enhancing blood oxygen levels, enhancing energy in healthy individuals, enhance working capacity and endurance, reducing muscle fatigue, reducing stress, enhancing cardiac and cardiovascular function, improving sexual ability, increasing muscle ATP levels, and/or reducing lactic acid in blood. In certain embodiments, the methods involve administering an amount of a sirtuin activating compound that increase mitochondrial activity, increase mitochondrial biogenesis, and/or increase mitochondrial mass.

Sports performance refers to the ability of the athlete's muscles to perform when participating in sports activities. Enhanced sports performance, strength, speed and endurance are measured by an increase in muscular contraction strength, increase in amplitude of muscle contraction, shortening of muscle reaction time between stimulation and contraction. Athlete refers to an individual who participates in sports at any level and who seeks to achieve an improved level of strength, speed and endurance in their performance, such as, for example, body builders, bicyclists, long distance runners, short distance runners, etc. Enhanced sports performance in manifested by the ability to overcome muscle fatigue, ability to maintain activity for longer periods of time, and have a more effective workout.

In the arena of athlete muscle performance, it is desirable to create conditions that permit competition or training at higher levels of resistance for a prolonged period of time.

It is contemplated that the methods of the present invention will also be effective in the treatment of muscle related pathological conditions, including acute sarcopenia, for example, muscle atrophy and/or cachexia associated with burns, bed rest, limb immobilization, or major thoracic, abdominal, and/or orthopedic surgery.

In certain embodiments, the invention provides novel dietary compositions comprising sirtuin modulators, a method for their preparation, and a method of using the compositions for improvement of sports performance. Accordingly, provided are therapeutic compositions, foods and beverages that have actions of improving physical endurance and/or inhibiting physical fatigues for those people involved in broadly-defined exercises including sports requiring endurance and labors requiring repeated muscle exertions. Such dietary compositions may additional comprise electrolytes, caffeine, vitamins, carbohydrates, etc.

### Other Uses

Sirtuin-modulating compounds that increase the level and/or activity of a sirtuin protein may be used for treating or preventing viral infections (such as infections by influenza, herpes or papilloma virus) or as antifungal agents. In certain embodiments, sirtuin-modulating compounds that increase the level and/or activity of a sirtuin protein may be administered as part of a combination drug therapy with another therapeutic agent for the treatment of viral diseases. In another embodiment, sirtuin-modulating compounds that increase the level and/or activity of a sirtuin protein may be administered as part of a combination drug therapy with another anti-fungal agent.

Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells. For example, modulating compounds may be administered to farm animals to improve their ability to withstand farming conditions longer.

Sirtuin-modulating compounds that increase the level and/or activity of a sirtuin protein may also be used to increase lifespan, stress resistance, and resistance to apoptosis in plants. In one embodiment, a compound is applied to plants, e.g., on a periodic basis, or to fungi. In another embodiment, plants are genetically modified to produce a compound. In another embodiment, plants and fruits are treated with a compound prior to picking and shipping to increase resistance to damage during shipping. Plant seeds may also be contacted with compounds described herein, e.g., to preserve them.

In other embodiments, sirtuin-modulating compounds that increase the level and/or activity of a sirtuin protein may be used for modulating lifespan in yeast cells. Situations in which it may be desirable to extend the lifespan of yeast cells include any process in which yeast is used, e.g., the making of beer, yoghurt, and bakery items, e.g., bread. Use of yeast having an extended lifespan can result in using less yeast or in having the yeast be active for longer periods of time. Yeast or other mammalian cells used for recombinantly producing proteins may also be treated as described herein.

Sirtuin-modulating compounds that increase the level and/or activity of a sirtuin protein may also be used to increase lifespan, stress resistance and resistance to apoptosis in insects. In this embodiment, compounds would be applied to useful insects, e.g., bees and other insects that are involved in pollination of plants. In a specific embodiment, a compound would be applied to bees involved in the production of honey. Generally, the methods described herein may be applied to any organism, e.g., eukaryote, that may have commercial importance. For example, they can be applied to fish (aquaculture) and birds (e.g., chicken and fowl).

Higher doses of sirtuin-modulating compounds that increase the level and/or activity of a sirtuin protein may also be used as a pesticide by interfering with the regulation of silenced genes and the regulation of apoptosis during development. In this embodiment, a compound may be applied to plants using a method known in the art that ensures the compound is bio-available to insect larvae, and not to plants.

At least in view of the link between reproduction and longevity, sirtuin-modulating compounds that increase the level and/or activity of a sirtuin protein can be applied to affect the reproduction of organisms such as insects, animals and microorganisms.

### 4. Assays

Various types of assays to determine sirtuin activity have been described. For example, sirtuin activity may be determined using a fluorescence based assay such as the assay commercially available from Biomol, e.g., the SIRT1 Fluorimetric Drug Discovery Kit (AK-555), SIRT2 Fluorimetric Drug Discovery Kit (AK-556), or SIRT3 Fluorimetric Drug Discovery Kit (AK-557) (Biomol International, Plymouth Meeting, Pa.). Other suitable sirtuin assays include a nicotinamide release assay (Kaeberlein et al., J. Biol. Chem. 280(17): 17038 (2005)), a FRET assay (Marcotte et al., Anal. Biochem. 332: 90 (2004)), and a C¹⁴ NAD boron resin binding assay (McDonagh et al., Methods 36: 346 (2005)). Yet other suitable sirtuin assays include radioimmunoassays (RIA), scintillation proximity assays, HPLC based assays, and reporter gene assays (e.g., for transcription factor targets).

An exemplary assay for determining sirtuin activity is a fluorescence polarization assay. Fluorescence polarization assays are described herein and are also described in PCT Publication No. WO 2006/094239. In other embodiments, sirtuin activity may be determined using a mass spectrometry based assays. Examples of mass spectrometry based assays are described herein and are also described in PCT Publication No. WO 2007/064902. Cell based assays may also be used to determine sirtuin activity. Examples of cell based assays for determining sirtuin activity are described in PCT Publication Nos. WO 2007/064902 and WO 2008/060400.

Yet other methods contemplated herein include screening methods for identifying compounds or agents that modulate sirtuins. An agent may be a nucleic acid, such as an aptamer. Assays may be conducted in a cell based or cell free format. For example, an assay may comprise incubating (or contacting) a sirtuin with a test agent under conditions in which a sirtuin can be modulated by an agent known to modulate the sirtuin, and monitoring or determining the level of modulation of the sirtuin in the presence of the test agent relative to the absence of the test agent. The level of modulation of a sirtuin can be determined by determining its ability to deacetylate a substrate. Exemplary substrates are acetylated peptides which can be obtained from BIOMOL (Plymouth Meeting, Pa.). Preferred substrates include peptides of p53, such as those comprising an acetylated K382. A particularly preferred substrate is the Fluor de Lys-SIRT1 (BIOMOL), i.e., the acetylated peptide Arg-His-Lys-Lys (SEQ ID NO: 2). Other substrates are peptides from human histones H3 and H4 or an acetylated amino acid. Substrates may be fluorogenic. The sirtuin may be SIRT1, Sir2, SIRT3, or a portion thereof. For example, recombinant SIRT1 can be obtained from BIOMOL. The reaction may be conducted for about 30 minutes and stopped, e.g., with nicotinamide. The HDAC fluorescent activity assay/drug discovery kit (AK-500, BIOMOL Research Laboratories) may be used to determine the level of acetylation. Similar assays are described in Bitterman et al. (2002) J. Biol. Chem. 277:45099. The level of modulation of the sirtuin in an assay may be compared to the level of modulation of the sirtuin in the presence of one or more (separately or simultaneously) compounds described herein, which may serve as positive or negative controls. Sirtuins for use in the assays may be full length sirtuin proteins or portions thereof. Since it has been shown herein that activating compounds appear to interact with the N-terminus of SIRT1, proteins for use in the assays include N-terminal portions of sirtuins, e.g., about amino acids 1-176 or 1-255 of SIRT1; about amino acids 1-174 or 1-252 of Sir2.

In one embodiment, a screening assay comprises (i) contacting a sirtuin with a test agent and an acetylated substrate under conditions appropriate for the sirtuin to deacetylate the substrate in the absence of the test agent; and (ii) determining the level of acetylation of the substrate, wherein a lower level of acetylation of the substrate in the presence of the test agent relative to the absence of the test agent indicates that the test agent stimulates deacetylation by the sirtuin, whereas a higher level of acetylation of the substrate in the presence of the test agent relative to the absence of the test agent indicates that the test agent inhibits deacetylation by the sirtuin.

Methods for identifying an agent that modulates, e.g., stimulates, sirtuins in vivo may comprise (i) contacting a cell with a test agent and a substrate that is capable of entering a cell in the presence of an inhibitor of class I and class II HDACs under conditions appropriate for the sirtuin to deacetylate the substrate in the absence of the test agent; and (ii) determining the level of acetylation of the substrate, wherein a lower level of acetylation of the substrate in the presence of the test agent relative to the absence of the test agent indicates that the test agent stimulates deacetylation by the sirtuin, whereas a higher level of acetylation of the substrate in the presence of the test agent relative to the absence of the test agent indicates that the test agent inhibits deacetylation by the sirtuin. A preferred substrate is an acetylated peptide, which is also preferably fluorogenic, as further described herein. The method may further comprise lysing the cells to determine the level of acetylation of the substrate. Substrates may be added to cells at a concentration ranging from about 1 μM to about 10 mM, preferably from about 10 μM to 1 mM, even more preferably from about 100 μM to 1 mM, such as about 200 μM. A preferred substrate is an acetylated lysine, e.g., ε-acetyl lysine (Fluor de Lys, FdL) or Fluor de Lys-SIRT1. A preferred inhibitor of class I and class II HDACs is trichostatin A (TSA), which may be used at concentrations ranging from about 0.01 to 100 μM, preferably from about 0.1 to 10 μM, such as Incubation of cells with the test compound and the substrate may be conducted for about 10 minutes to 5 hours, preferably for about 1-3 hours. Since TSA inhibits all class I and class II HDACs, and that certain substrates, e.g., Fluor de Lys, is a poor substrate for SIRT2 and even less a substrate for SIRT3-7, such an assay may be used to identify modulators of SIRT1 in vivo.

### 5. Pharmaceutical Compositions

The sirtuin-modulating compounds described herein may be formulated in a conventional manner using one or more physiologically acceptable carriers or excipients. For example, sirtuin-modulating compounds and their physiologically acceptable salts and solvates may be formulated for administration by, for example, injection (e.g. SubQ, IM, IP), inhalation or insufflation (either through the mouth or the nose) or oral, buccal, sublingual, transdermal, nasal, parenteral or rectal administration. In one embodiment, a sirtuin-modulating compound may be administered locally, at the site where the target cells are present, i.e., in a specific tissue, organ, or fluid (e.g., blood, cerebrospinal fluid, etc.).

Sirtuin-modulating compounds can be formulated for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation (e.g., pulmonary delivery), sirtuin-modulating compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

Sirtuin-modulating compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Sirtuin-modulating compounds may also be formulated in rectal compositions such as suppositories or retention enemas, e.g., containing conventional suppository bases such as cocoa butter or other glycerides.

In addition to the formulations described previously, sirtuin-modulating compounds may also be formulated as a depot preparation. Such long acting formulations may be administered by implantation (for example subcutaneously or intramuscularly) or by intramuscular injection. Thus, for example, sirtuin-modulating compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

In certain embodiments, the compounds described herein can be formulated for delivery to the central nervous system (CNS) (reviewed in Begley, Pharmacology & Therapeutics 104: 29-45 (2004)). Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

Liposomes are a further drug delivery system which is easily injectable. Accordingly, in the method of invention the active compounds can also be administered in the form of a liposome delivery system. Liposomes are well-known by a person skilled in the art. Liposomes can be formed from a variety of phospholipids, such as cholesterol, stearylamine of phosphatidylcholines. Liposomes being usable for the method of invention encompass all types of liposomes including, but not limited to, small unilamellar vesicles, large unilamellar vesicles and multilamellar vesicles.

Another way to produce a formulation, particularly a solution, of a sirtuin modulator such as resveratrol or a derivative thereof, is through the use of cyclodextrin. By cyclodextrin is meant α-, β-, or γ-cyclodextrin. Cyclodextrins are described in detail in Pitha et al., U.S. Pat. No. 4,727,064, which is incorporated herein by reference. Cyclodextrins are cyclic oligomers of glucose; these compounds form inclusion complexes with any drug whose molecule can fit into the lipophile-seeking cavities of the cyclodextrin molecule.

Rapidly disintegrating or dissolving dosage forms are useful for the rapid absorption, particularly buccal and sublingual absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

Pharmaceutical compositions (including cosmetic preparations) may comprise from about 0.00001 to 100% such as from 0.001 to 10% or from 0.1% to 5% by weight of one or more sirtuin-modulating compounds described herein.

In one embodiment, a sirtuin-modulating compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Sirtuin-modulating compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing.

Sirtuin-modulating compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type.

Sirtuin-modulating compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Sirtuin-modulating compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9).

Sirtuin-modulating compounds may be incorporated into gel formulations, which generally are semisolid systems consisting of either suspensions made up of small inorganic particles (two-phase systems) or large organic molecules distributed substantially uniformly throughout a carrier liquid (single phase gels). Although gels commonly employ aqueous carrier liquid, alcohols and oils can be used as the carrier liquid as well.

Other active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Conditions of the eye can be treated or prevented by, e.g., systemic, topical, intraocular injection of a sirtuin-modulating compound, or by insertion of a sustained release device that releases a sirtuin-modulating compound. A sirtuin-modulating compound that increases the level and/or activity of a sirtuin protein may be delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, the compounds of the invention may be injected directly into the vitreous and aqueous humour. In a further alternative, the compounds may be administered systemically, such as by intravenous infusion or injection, for treatment of the eye.

Sirtuin-modulating compounds described herein may be stored in oxygen free environment. For example, resveratrol or analog thereof can be prepared in an airtight capsule for oral administration, such as Capsugel from Pfizer, Inc.

Cells, e.g., treated ex vivo with a sirtuin-modulating compound, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Toxicity and therapeutic efficacy of sirtuin-modulating compounds can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Sirtuin-modulating compounds that exhibit large therapeutic indexes are preferred. While sirtuin-modulating compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may lie within a range of circulating concentrations that include the ED50 with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### 6. Kits

Also provided herein are kits, e.g., kits for therapeutic purposes or kits for modulating the lifespan of cells or modulating apoptosis. A kit may comprise one or more sirtuin-modulating compounds, e.g., in premeasured doses. A kit may optionally comprise devices for contacting cells with the compounds and instructions for use. Devices include syringes, stents and other devices for introducing a sirtuin-modulating compound into a subject (e.g., the blood vessel of a subject) or applying it to the skin of a subject.

In yet another embodiment, the invention provides a composition of matter comprising a sirtruin modulator of this invention and another therapeutic agent (the same ones used in combination therapies and combination compositions) in separate dosage forms, but associated with one another. The term “associated with one another” as used herein means that the separate dosage forms are packaged together or otherwise attached to one another such that it is readily apparent that the separate dosage forms are intended to be sold and administered as part of the same regimen. The agent and the sirtruin modulator are preferably packaged together in a blister pack or other multi-chamber package, or as connected, separately sealed containers (such as foil pouches or the like) that can be separated by the user (e.g., by tearing on score lines between the two containers).

In still another embodiment, the invention provides a kit comprising in separate vessels, a) a sirtruin modulator of this invention; and b) another therapeutic agent such as those described elsewhere in the specification.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### Preparation of N-(2-chloropyridin-3-yl)-2-nitrobenzamide

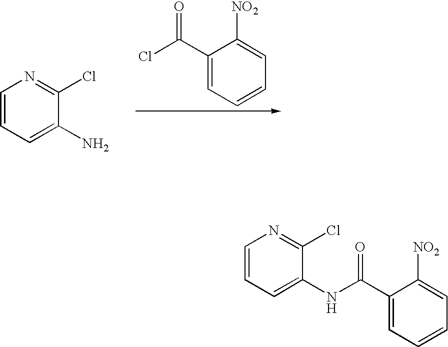

A mixture of 3-amino-2-chloropyridine (3.85 g, 29.95 mmol) and 2-nitrobenzoyl chloride (5.56 g, 29.95 mmol) in pyridine (50 mL) was stirred at 0° C. for 1 h and then at room temperature overnight. Water was added and the precipitate formed was collected by filtration and dried to give N-(2-chloropyridin-3-yl)-2-nitrobenzamide as a white solid (8.52 g, crude yield: >100%).

### Preparation of 2-(thiazolo[5,4-b]pyridin-2-yl)aniline

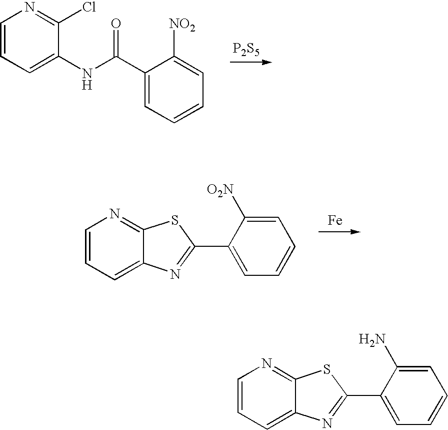

A mixture of N-(2-chloropyridin-3-yl)-2-nitrobenzamide (12.98 g, 46.75 mmol), P₂S₅ (31.17 g, 140.24 mmol) and pyridine (80 mL) in p-xylene (310 mL) was heated at 120° C. for 18 hours. Stirring was discontinued for 30 min, and the mixture was cooled to 100° C. The upper clear solution was transferred and concentrated in vacuo, followed by the addition of ethanol (50 mL). The suspension was heated at 75° C. for 30 min to dissolve the product, filtered while hot, cooled to room temperature and left standing 18 hours. The solid was collected by filtration, washed with cold ethanol, and dried in vacuo to give a crude mixture of N-(2-chloropyridin-3-yl)-2-nitrobenzamide and 2-(2-nitrophenyl)thiazolo[5,4-b]pyridine as a yellow solid (10.60 g).

The above crude mixture (10.60 g), iron (11.50 g, 206.01 mmol), and NH₄Cl (17.63 g, 329.61 mmol) in methanol (MeOH)/H₂O (80/20 mL) was heated at reflux for 2 hours. The reaction mixture was cooled to room temperature and extracted with ethyl acetate. The organic layer was concentrated in vacuo and purified by chromatography on silica gel to give 2-(thiazolo[5,4-b]pyridin-2-yl)aniline as a yellow solid (3 g, 28% yield over two steps). (MS, M⁺+H=228).

### Preparation of 6-hydroxy-2-phenylpyrimidine-4-carboxylic acid

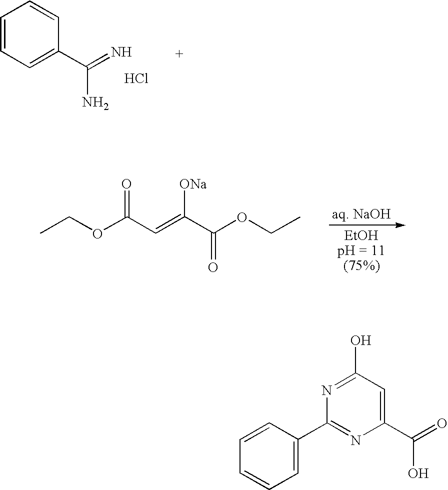

Diethyl oxaloacetate sodium salt 4.84 g (23.0 mmol) was added to a solution of 16 mL of water and 4 mL of ethanol. The suspension was stirred for 5 min, then a solution of 3.6 mL (22.5 mmol) of 6.25 M NaOH_{(aq.)} was added. The mixture was stirred at ambient temperature for 15 minutes to give a tan solution. To this was added a solution of 3.01 g (19.2 mmol) of benzamidine hydrochloride in 15 mL of H₂O, giving a solution with pH=11. Next, 1 mL of 6.25 M NaOH was added, pH=11 when done, then the reaction was stirred at 80° C. for 2 h. Additional NaOH was added during the heating period to maintain the pH between 11 and 12. (A total of about 10 mmol additional NaOH was added.) The reaction was cooled to 5° C., then 12M HCl was added until pH=1. The white precipitate was collected, washed with water, and dried on the filter to give 3.09 g (74%) of the product as a white solid.

### Preparation of 6-chloro-2-phenylpyrimidine-4-carbonyl chloride

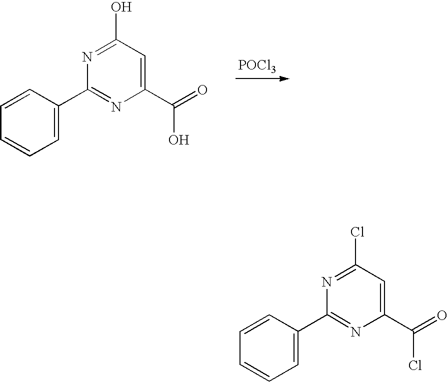

To 1.00 g of 6-hydroxy-2-phenylpyrimidine-4-carboxylic acid was added 10 mL of phosphorus oxychloride. The reaction was heated at reflux for 1 h, then concentrated in vacuo to an oil, removing as much phosphorus oxychloride as possible. The oil was suspended in 30 mL of pentane, then the mixture was extracted with water (3×5 mL) and brine (1×5 mL). The organic layer was dried over MgSO₄, filtered, and concentrated in vacuo to give 1.03 g (88%) of the acid chloride as a white solid.

### Preparation of 6-chloro-2-phenyl-N-(2-(thiazolo[5,4-b]pyridin-2-yl)phenyl)pyrimidine-4-carboxamide

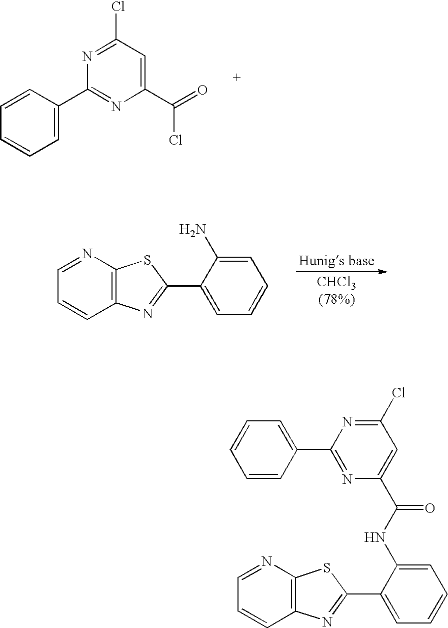

To a solution of 368 mg (1.62 mmol) of 2-(thiazolo[5,4-b]pyridin-2-yl)aniline in 10 mL of chloroform was added 500 μL of N,N-diisopropylethylamine (i.e., Hunig's base). To this was added a solution of between 1.6 and 2 mmol of 6-chloro-2-phenylpyrimidine-4-carbonyl chloride in 5 mL of chloroform, then the reaction was stirred at ambient temperature. The product began to crystallize within a few minutes. After 30 minutes, the reaction was diluted with 50 mL of methanol, then the precipitate was filtered, washed with additional methanol, and dried on the filter to give 561 mg (78%) of the amide as a yellow solid (MS, M⁺+H=444).

### General Method A

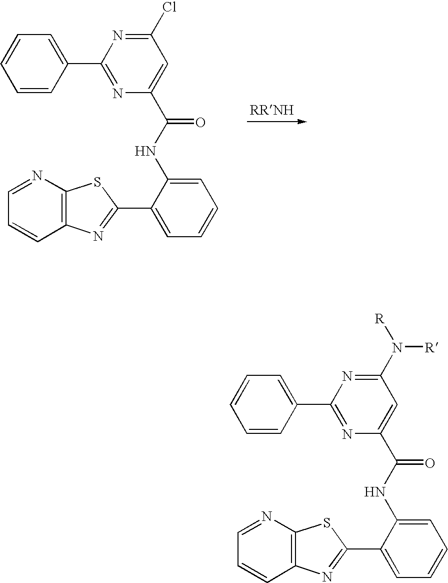

To a suspension of 50 mg (0.11 mmol) of 6-chloro-2-phenyl-N-(2-(thiazolo[5,4-b]pyridin-2-yl)phenyl)pyrimidine-4-carboxamide and 1 mL of tetrahydrofuran was added ca. 1 mmol of the amine. The suspension was stirred at reflux for 30 min, during which time dissolution occurred. Heating was removed, and the reaction was diluted with 5 mL of water. The precipitate was filtered, washed with additional water, then acetonitrile, and dried on the filter to give a solid. If impure, products were recrystallized.

### Preparation of 2-Phenyl-6-piperazin-1-yl-pyrimidine-4-carboxylic acid (2-thiazolo[5,4-b]pyridin-2-yl-phenyl)-amide hydrochloride

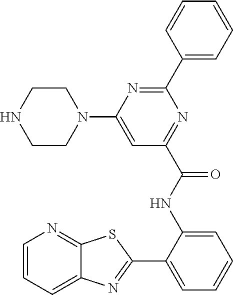

The title compound was prepared according to general method A. The intermediate 4-[2-Phenyl-6-(2-thiazolo[5,4-b]pyridin-2-yl-phenylcarbamoyl)-pyrimidin-4-yl]piperazine-1-carboxylic acid tert-butyl ester was treated with 20% trifluoroacetic acid (TFA) in dichloromethane (DCM) for 1 h. The solvent was evaporated and the residue diluted with acetonitrile/water 1:4. Aqueous HCl (1N) was added (2.5 equiv) and the resulting solution was lyophilized to afford a slightly sticky solid. The solid was taken up in acetonitrile/water 1:4. 1N aqueous HCl was added (1 equiv) and the resulting solution was lyophilized again to afford 92% overall yield of the title compound as a free flowing yellow solid (MS, M⁺+H=494).

### Preparation of 6-(4-(2-methoxyethyl)piperazin-1-yl)-2-phenyl-N-(2-(thiazolo[5,4-b]pyridin-2-yl)phenyl)pyrimidine-4-carboxamide

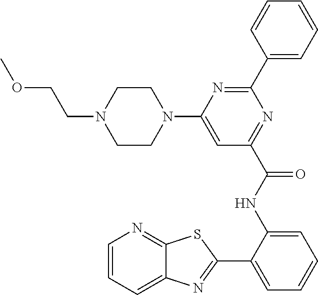

Prepared by general method A. (MS, M⁺+H=552).

### Preparation of 6-(2-(dimethylamino)ethylamino)-2-phenyl-N-(2-(thiazolo[5,4-b]pyridin-2-yl)phenyl)pyrimidine-4-carboxamide

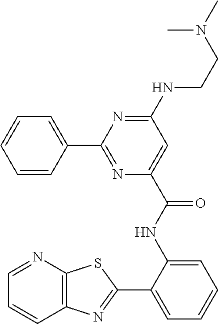

Prepared by general method A. Recrystallized from ethanol to give 40 mg (71%) of a yellow solid. (MS, M⁺+H=496).

### Preparation of 6-(2-methoxyethylamino)-2-phenyl-N-(2-(thiazolo[5,4-b]pyridin-2-yl)phenyl)pyrimidine-4-carboxamide

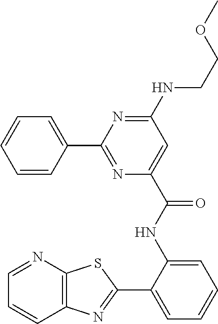

Prepared by general method A. Recrystallized from ethanol to give 39 mg (72%) of a white solid. (MS, M⁺+H=483).

### Preparation of 2-phenyl-N-(2-(thiazolo[5,4-b]pyridin-2-yl)phenyl)-6-((1,1-dioxo)thiomorpholino)pyrimidine-4-carboxamide

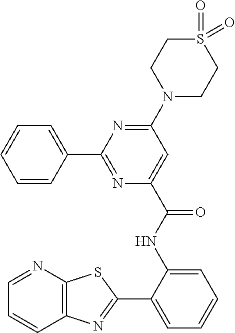

Prepared by general method A. Triturated with hot ethanol to give 44 mg (72%) of a white solid. (MS, M⁺+H=543).

### Preparation of methyl 2-(2-phenyl-6-(2-(thiazolo[5,4-b]pyridin-2-yl)phenylcarbamoyl)pyrimidin-4-ylamino)acetate

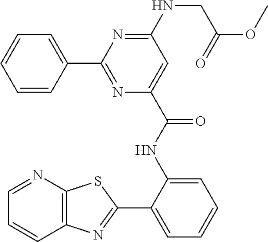

To a mixture of 200 mg (0.451 mmol) of 6-chloro-2-phenyl-N-(2-(thiazolo[5,4-b]pyridin-2-yl)phenyl)pyrimidine-4-carboxamide and 142 mg (1.13 mmol) of glycine methyl ester hydrochloride was added 4 mL of dimethylsulfoxide (DMSO) and 400 μL (2.30 mmol) of Hunig's base. The mixture was stirred at 120° C. for 20 min, then the reaction was removed from heating and diluted with 35 mL of CH₃OH. The precipitate was filtered, washed with additional CH₃OH, and dried on the filter to give 224 mg (78%) of an off-white powder. (MS, M⁺+H=497).

### Preparation of 6-Cyclopentylamino-2-phenyl-pyrimidine-4-carboxylic acid (2-thiazolo[5,4-b]pyridin-2-yl-phenyl)-amide

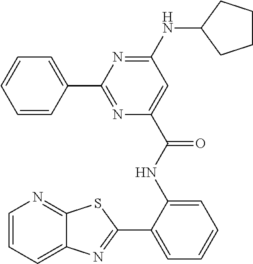

The title compound was prepared in 77% yield according to general method A. (MS, M⁺+H=493).

### Preparation of 2-Phenyl-6-piperidin-1-yl-pyrimidine-4-carboxylic acid (2-thiazolo[5,4-b]pyridin-2-yl-phenyl)-amide

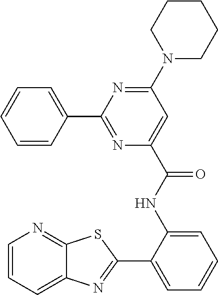

The title compound was prepared in 68% yield according to general method A. (MS, M⁺+H=493).

### Preparation of 6-((3R,5S)-3,5-Dimethyl-piperazin-1-yl)-2-phenyl-pyrimidine-4-carboxylic acid (2-thiazolo[5,4-b]pyridin-2-yl-phenyl)-amide

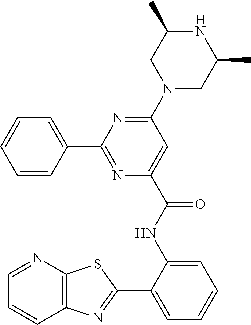

The title compound was prepared in 86% yield according to general method A. (MS, M⁺+H=522).

### Preparation of 6-[1,4]Diazepan-1-yl-2-phenyl-pyrimidine-4-carboxylic acid (2-thiazolo[5,4-b]pyridin-2-yl-phenyl)-amide hydrochloride

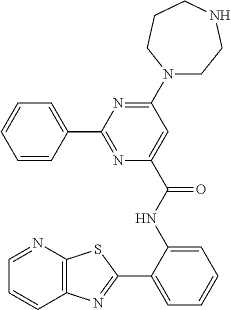

The title compound was prepared according to general method A followed by treatment with 20% TFA in DCM. The solvent was evaporated, the residue was taken up in acetonitrile/water 1:5, 1N aqueous HCl (6 equiv) was added and the resulting gel-like suspension was lyophilized to afford quantitative yield of the title compound (MS, M⁺+H=508).

### Preparation of ethyl 3-(2-phenyl-6-(2-(thiazolo[5,4-b]pyridin-2-yl)phenylcarbamoyl)pyrimidin-4-ylamino)propanoate

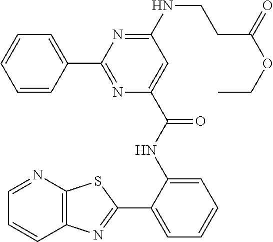

Prepared according to the procedure employed for methyl 2-(2-phenyl-6-(2-(thiazolo[5,4-b]pyridin-2-yl)phenylcarbamoyl)pyrimidin-4-ylamino)acetate, substituting β-alanine ethyl ester hydrochloride for glycine methyl ester hydrochloride. Recrystallized from ethanol to give 53 mg (74%) of yellow crystals. (MS, M⁺+H=525).

### Preparation of 3-(2-phenyl-6-(2-(thiazolo[5,4-b]pyridin-2-yl)phenylcarbamoyl)pyrimidin-4-ylamino)propanoic acid

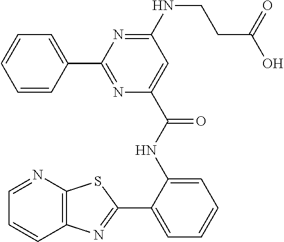

To a solution of 24 mg (0.046 mmol) of ethyl 3-(2-phenyl-6-(2-(thiazolo[5,4-b]pyridin-2-yl)phenylcarbamoyl)pyrimidin-4-ylamino)propanoate in 1 mL of DMSO was added 0.1 mL of 2M NaOH_{(aq.)}. The yellow solution was stirred at ambient temperature for 25 min, then 10 mL of water was added, followed by 1 mL of 1 M HCl. The suspension was extracted with ethyl acetate (3×5 mL), then the suspension of the product in the ethyl acetate layer was back extracted with water (1×5 mL). The organic layer was heated to dissolve the product, extracted with brine (1×5 mL), dried over MgSO₄, filtered, and concentrated to 16 mg (70%) of a white solid. (MS, M⁺+H=497).

### Preparation of 6-[(2-Methoxy-ethyl)-methyl-amino]-2-phenyl-pyrimidine-4-carboxylic acid (2-thiazolo[5,4-b]pyridin-2-yl-phenyl)-amide

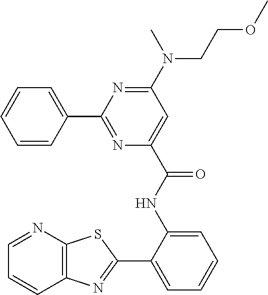

The title compound was prepared in 99% yield according to general method A. (MS, M⁺+H=497).

### Preparation of 2-Phenyl-6-(2-piperazin-1-yl-ethylamino)-pyrimidine-4-carboxylic acid (2-thiazolo[5,4-b]pyridin-2-yl-phenyl)-amide hydrochloride

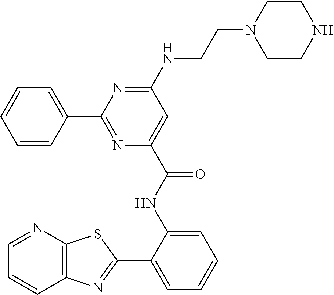

The title compound was prepared according to general method A followed by treatment with 20% TFA in DCM. The solvent was evaporated, the residue was taken up in acetonitrile/water 1:2.5, 1N aqueous HCl (20 equiv) was added and the solution was lyophilized to afford quantitative yield of the title compound (MS, M⁺+H=537).

### Preparation of 2-Phenyl-6-(piperidin-4-ylamino)-pyrimidine-4-carboxylic acid (2-thiazolo[5,4-b]pyridin-2-yl-phenyl)-amide hydrochloride

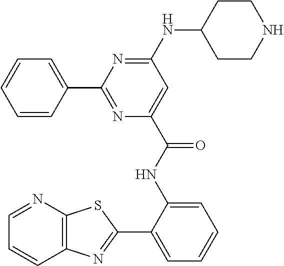

The title compound was prepared according to general method A followed by treatment with 20% TFA in DCM. The solvent was evaporated, the residue was taken up in acetonitrile/water 1:2.5, 1N aqueous HCl (20 equiv) was added and the resulting solution was lyophilized to afford quantitative yield of the title compound (MS, M⁺+H=508).

### Preparation of 6-(2-Acetylamino-ethylamino)-2-phenyl-pyrimidine-4-carboxylic acid (2-thiazolo[5,4-b]pyridin-2-yl-phenyl)-amide

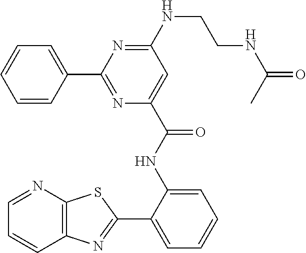

The title compound was prepared in 52% yield according to general method A. (MS, M⁺+H=510).

### Preparation of 6-(2-Isopropylamino-ethylamino)-2-phenyl-pyrimidine-4-carboxylic acid (2-thiazolo[5,4-b]pyridin-2-yl-phenyl)-amide hydrochloride

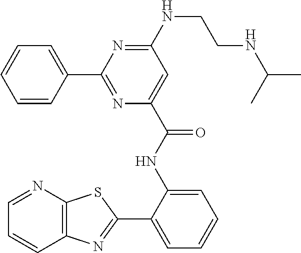

The title compound was prepared in 39% overall yield according to general method A. Treatment of the free base with DCM (2 mL) and 4M HCl in methanol (1 mL) gave a precipitate. This was filtered, washed with DCM, and dried in vacuo to give the HCl salt (135 mg) (MS, M⁺+H=510).

### Preparation of 6-ethoxy-2-phenyl-N-(2-(thiazolo[5,4-b]pyridin-2-yl)phenyl)pyrimidine-4-carboxamide

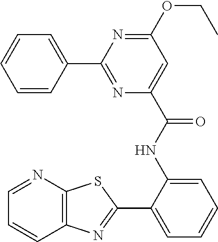

To 50 mg (0.11 mmol) of 6-chloro-2-phenyl-N-(2-(thiazolo[5,4-b]pyridin-2-yl)phenyl)pyrimidine-4-carboxamide was added 1 mL of DMSO. To this was added 0.2 mL (0.5 mmol) of 2.54 M potassium ethoxide in ethanol. The reaction was heated at 120° C. for 10 min, then cooled to ambient temperature and diluted with 10 mL of methanol. The precipitate was filtered, washed with additional methanol, and dried on the filter to give 29 mg (57%) of a white solid. (MS, M⁺+H=454).

### Preparation of 6-(isobutylamino)-2-phenyl-N-(2-(thiazolo[5,4-b]pyridin-2-yl)phenyl)pyrimidine-4-carboxamide

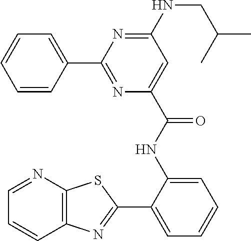

Prepared in 75% yield by general method A. (MS, M⁺+H=481).

### Preparation of 6-(dimethylamino)-2-phenyl-N-(2-(thiazolo[5,4-b]pyridin-2-yl)phenyl)pyrimidine-4-carboxamide

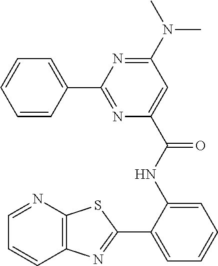

Prepared by general method A. 132 mg (60% yield). (MS, M⁺+H=453).

### Preparation of 6-(2-hydroxyethylamino)-2-phenyl-N-(2-(thiazolo[5,4-b]pyridin-2-yl)phenyl)pyrimidine-4-carboxamide

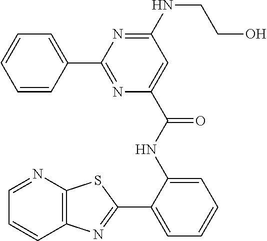

Prepared by general method A. Recrystallized from ethanol to give 44 mg (83%) of a white solid. (MS, M⁺+H=469).

### Preparation of 6-(3-hydroxypropylamino)-2-phenyl-N-(2-(thiazolo[5,4-b]pyridin-2-yl)phenyl)pyrimidine-4-carboxamide

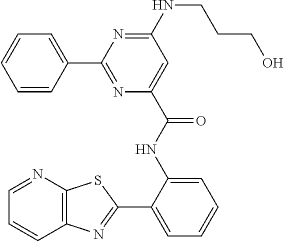

Prepared by general method A. Recrystallized from ethanol to give 29 mg (53%) of a white solid. (MS, M⁺+H=483).

### Preparation of 6-(2-(diethylamino)ethylamino)-2-phenyl-N-(2-(thiazolo[5,4-b]pyridin-2-yl)phenyl)pyrimidine-4-carboxamide

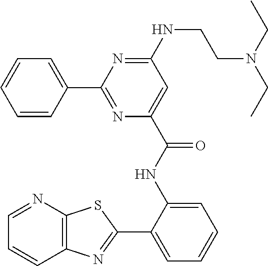

Prepared by general method A. Recrystallized from ethanol to give 40 mg (68%) of a white solid. (MS, M⁺+H=524).

### Preparation of 6-(butylamino)-2-phenyl-N-(2-(thiazolo[5,4-b]pyridin-2-yl)phenyl)pyrimidine-4-carboxamide

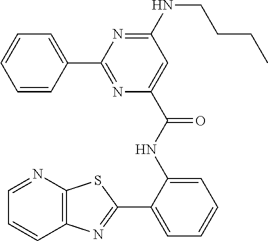

Prepared by general method A. Recrystallized from ethanol to give 36 mg (66%) of a white solid. (MS, M⁺+H=481).

### Preparation of 6-(2-(methylthio)ethylamino)-2-phenyl-N-(2-(thiazolo[5,4-b]-pyridin-2-yl)phenyl)pyrimidine-4-carboxamide

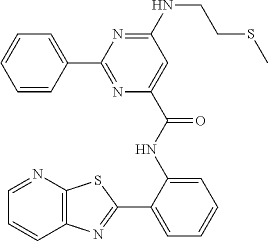

Prepared by general method A. 97 mg (86%), white solid. (MS, M⁺+H=499).

### Preparation of 6-(2-(methylsulfonyl)ethylamino)-2-phenyl-N-(2-(thiazolo[5,4-b]pyridin-2-yl)phenyl)pyrimidine-4-carboxamide

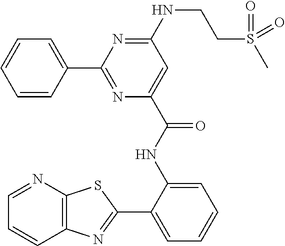

To a solution of 50 mg (0.10 mmol) of 6-(2-(methylthio)ethylamino)-2-phenyl-N-(2-(thiazolo[5,4-b]pyridin-2-yl)phenyl)pyrimidine-4-carboxamide in 1 mL of trifluoroacetic acid was added 50 mL of 30% (9.8M) H₂O₂. The reaction was stirred at ambient temperature for 1 h, then diluted with 10 mL of water. The precipitate was filtered, washed with water, and dried on the filter to give 43 mg (81%) of a pale yellow solid. (MS, M⁺+H=531).

### Preparation of 6-[2-(2-Methyl-thiazol-4-yl)-ethylamino]-2-phenyl-pyrimidine-4-carboxylic acid (2-thiazolo[5,4-b]pyridin-2-yl-phenyl)-amide

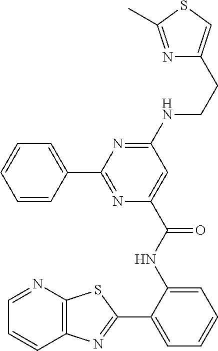

The title compound was prepared in 66% yield according to general method A (MS, M⁺+H=550).

### Preparation of 6-Benzylamino-2-phenyl-pyrimidine-4-carboxylic acid (2-thiazolo[5,4-b]pyridin-2-yl-phenyl)-amide

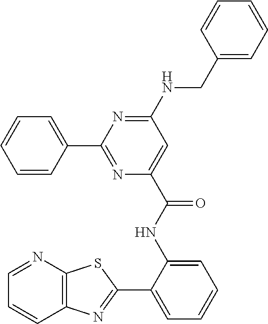

The title compound was prepared in 70% yield according to general method A (MS, M⁺+H=515).

### Preparation of 2-Phenyl-6-[(pyridin-2-ylmethyl)-amino]-pyrimidine-4-carboxylic acid (2-thiazolo[5,4-b]pyridin-2-yl-phenyl)-amide hydrochloride

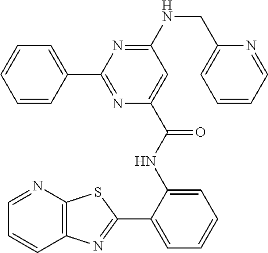

The title compound was prepared in 70% overall yield according to general method A. Treatment of the free base with DCM (2 mL) and 4M HCl in methanol (1 mL) gave a precipitate. This was filtered, washed with DCM, and dried in vacuo to give the HCl salt (202 mg) (MS, M⁺+H=516).

### Preparation of 2-Phenyl-6-[(pyridin-3-ylmethyl)-amino]pyrimidine-4-carboxylic acid (2-thiazolo[5,4-b]pyridin-2-yl-phenyl)-amide hydrochloride

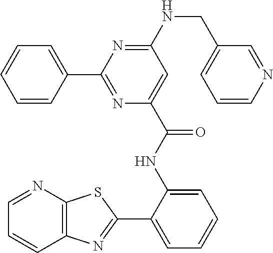

The title compound was prepared in 56% overall yield according to general method A. Treatment of the free base with DCM (2 mL) and 4 M HCl in methanol (1 mL) gave a precipitate. This was filtered, washed with DCM, and dried in vacuo to give the HCl salt (170 mg) (MS, M⁺+H=516).

### Preparation of 2-Phenyl-6-[(pyridin-4-ylmethyl)-amino]-pyrimidine-4-carboxylic acid (2-thiazolo[5,4-b]pyridin-2-yl-phenyl)-amide hydrochloride

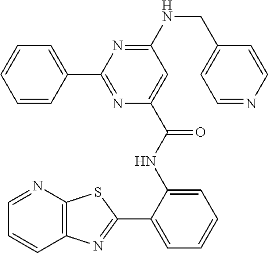

The title compound was prepared in 67% overall yield according to general method A. Treatment of the free base with DCM (2 mL) and 4M HCl in methanol (1 mL) gave a precipitate. This was filtered, washed with DCM, and dried in vacuo to give the HCl salt (186 mg) (MS, M⁺+H=516).

### Preparation of 2-Phenyl-6-(2-phenylamino-ethylamino)-pyrimidine-4-carboxylic acid (2-thiazolo[5,4-b]pyridin-2-yl-phenyl)-amide hydrochloride

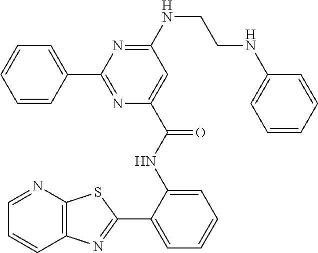

The title compound was prepared in 37% overall yield according to general method A. Treatment of the free base with DCM (2 mL) and 4M HCl in methanol (1 mL) gave a precipitate. This was filtered, washed with DCM, and dried in vacuo to give the HCl salt (117 mg) (MS, M⁺+H=544).

### Preparation of 6-(2-morpholinoethylamino)-2-phenyl-N-(2-(thiazolo[5,4-b]pyridin-2-yl)phenyl)pyrimidine-4-carboxamide hydrochloride

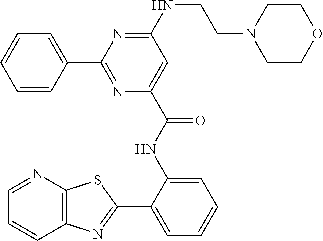

Prepared by general method A. Treatment of the free base with 2 mL of CH₂Cl₂ and 1 mL 4M HCl in methanol gave a precipitate. This was filtered, washed with CH₂Cl₂, and dried in vacuo to give 255 mg (86%) of the HCl salt. (MS, M⁺+H=538).

### Preparation of 2-phenyl-6-(2-(pyrrolidin-1-yl)ethylamino)-N-(2-(thiazolo[5,4-b]pyridin-2-yl)phenyl)pyrimidine-4-carboxamide hydrochloride

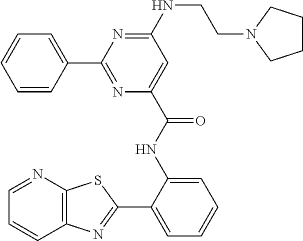

Prepared by general method A. Treatment of the free base with 2 mL of CH₂Cl₂ and 1 mL 4M HCl in methanol gave a precipitate. This was filtered, washed with CH₂Cl₂, and dried in vacuo to give 232 mg (85%) of the HCl salt. (MS, M⁺+H=522).

### Preparation of 6-(cyclohexylamino)-2-phenyl-N-(2-(thiazolo[5,4-b]pyridin-2-yl)phenyl)pyrimidine-4-carboxamide

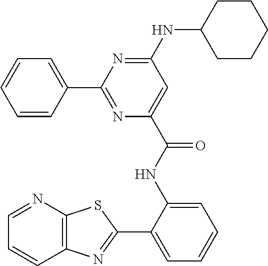

Prepared by general method A. Yield 220 mg (81%). (MS, M⁺+H=507).

### Preparation of 6-(methylamino)-2-phenyl-N-(2-(thiazolo[5,4-b]pyridin-2-yl)phenyl)pyrimidine-4-carboxamide

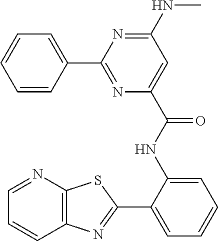

Prepared by general method A. Yield 180 mg (84%). (MS, M⁺+H=439).

### Preparation of 6-(4-isopropylpiperazin-1-yl)-2-phenyl-N-(2-(thiazolo[5,4-b]pyridin-2-yl)phenyl)pyrimidine-4-carboxamide hydrochloride

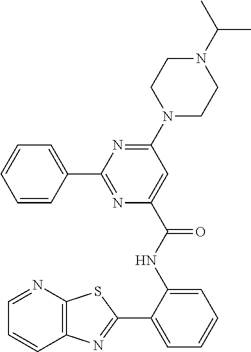

Prepared by general method A. Treatment of the free base with 2 mL of CH₂Cl₂ and 1 mL 4M HCl in methanol gave a precipitate. This was filtered, washed with CH₂Cl₂, and dried in vacuo to give 227 mg (80%) of the HCl salt. (MS, M⁺+H=536).

### Preparation of 6-(2-(4-hydroxypiperidin-1-yl)ethylamino)-2-phenyl-N-(2-(thiazolo[5,4-b]pyridin-2-yl)phenyl)pyrimidine-4-carboxamide hydrochloride

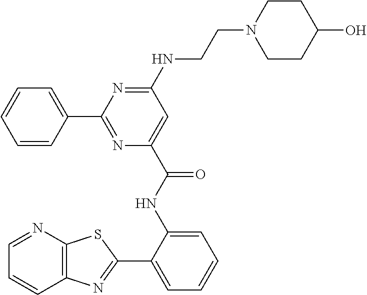

Prepared by general method A. Treatment of the free base with 2 mL of CH₂Cl₂ and 1 mL 4M HCl in methanol gave a precipitate. This was filtered, washed with CH₂Cl₂, and dried in vacuo to give 283 mg (62%) of the HCl salt. (MS, M⁺+H=552).

### Preparation of 2-phenyl-6-(2-(piperidin-1-yl)ethylamino)-N-(2-(thiazolo[5,4-b]pyridin-2-yl)phenyl)pyrimidine-4-carboxamide

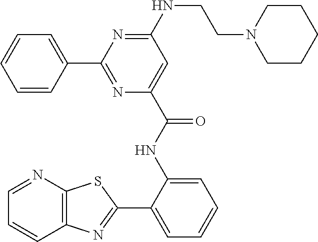

Prepared by general method A. Treatment of the free base with 2 mL of CH₂Cl₂ and 1 mL 4M HCl in methanol gave a precipitate. This was filtered, washed with CH₂Cl₂, and dried in vacuo to give 277 mg (89%) of the HCl salt. (MS, M⁺+H=536).

### Preparation of 6-(bis(2-methoxyethyl)amino)-2-phenyl-N-(2-(thiazolo[5,4-b]pyridin-2-yl)phenyl)pyrimidine-4-carboxamide

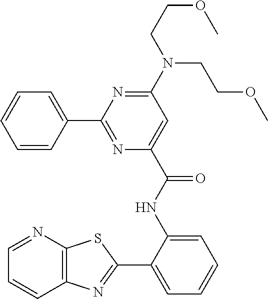

Prepared by general method A. Yield 180 mg (62%). (MS, M⁺+H=541).

### Preparation of 6-(2-Hydroxy-1-hydroxymethyl-ethylamino)-2-phenyl-pyrimidine-4-carboxylic acid (2-thiazolo[5,4-b]pyridin-2-yl-phenyl)-amide

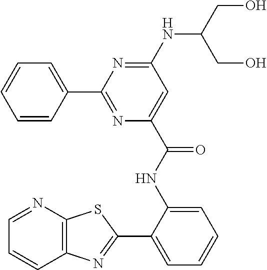

The title compound was prepared in 64% yield according to general method A. (MS, M⁺+H=499).

### Preparation of 6-((2R,6S)-2,6-Dimethyl-morpholin-4-yl)-2-phenyl-pyrimidine-4-carboxylic acid (2-thiazolo[5,4-b]pyridin-2-yl-phenyl)-amide

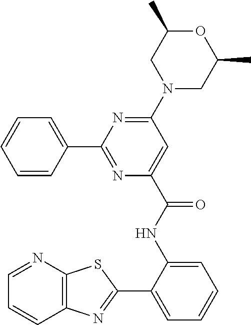

The title compound was prepared in quantitative yield according to general method A. MS, M⁺+H=523).

### Preparation of 6-(1R,4R)-2,5-Diaza-bicyclo[2.2.1]hept-2-yl-2-phenyl-pyrimidine-4-carboxylic acid (2-thiazolo[5,4-b]pyridin-2-yl-phenyl)-amide hydrochloride

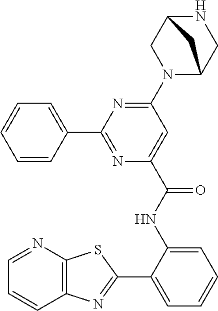

The title compound was prepared in 90% overall yield according to general method A, followed by treatment with 20% TFA in DCM. The solvent was evaporated, the residue was taken up in acetonitrile/water 1:2.5, 1N aqueous HCl (30 equiv) was added and the resulting solution was lyophilized to afford the title compound as a yellow solid (MS, M⁺+H=506).

### Preparation of 2-Phenyl-6-(piperidin-3-ylamino)-pyrimidine-4-carboxylic acid (2-thiazolo[5,4-b]pyridin-2-yl-phenyl)-amide

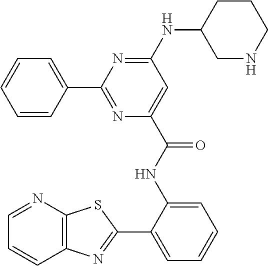

The title compound was prepared in 83% overall yield according to general method A. followed by treatment with 20% TFA in DCM. The solvent was evaporated, the residue was taken up in acetonitrile/water 1:2.5, and basified with 1N aqueous NaOH to pH=8. The product precipitated and was filtered, washed with water and acetonitrile and air dried to afford the title compound as a white powder (MS, M⁺+H=508).

### Preparation of 6-(2-(ethylthio)ethylamino)-2-phenyl-N-(2-(thiazolo[5,4-b]pyridin-2-yl)phenyl)pyrimidine-4-carboxamide

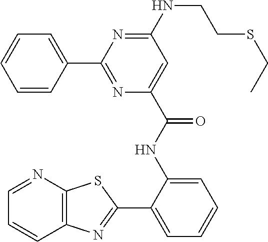

Prepared by general method A. Triturated with hot methanol to give 69 mg (60%) of a pale yellow solid. (MS, M⁺+H=513).

### Preparation of 6-(2-(ethylsulfonyl)ethylamino)-2-phenyl-N-(2-(thiazolo[5,4-b]pyridin-2-yl)phenyl)pyrimidine-4-carboxamide

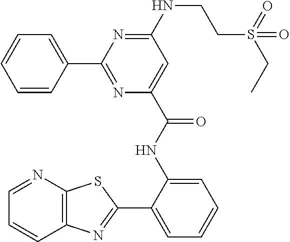

To a solution of 50 mg (0.097 mmol) of 6-(2-(ethylthio)ethylamino)-2-phenyl-N-(2-(thiazolo[5,4-b]pyridin-2-yl)phenyl)pyrimidine-4-carboxamide in 1 mL of trifluoroacetic acid was added 50 μL (0.40 mmol) of 30% (9.8M) H₂O₂. (MS, M⁺+H=545).

### Preparation of (S)-2-(2-phenyl-6-(2-(thiazolo[5,4-b]pyridin-2-yl)phenylcarbamoyl)pyrimidin-4-ylamino)propanoic acid

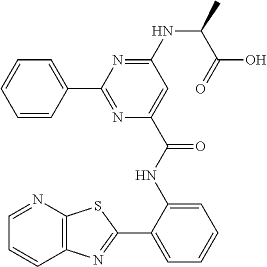

To a mixture of 200 mg (0.451 mmol) of 6-chloro-2-phenyl-N-(2-(thiazolo[5,4-b]pyridin-2-yl)phenyl)pyrimidine-4-carboxamide, 160 mg (1.15 mmol) of L-alanine methyl ester hydrochloride, and 400 μL (2.3 mmol) of Hunig's base was added 4 mL of DMSO. The reaction was heated at 90° C. for 3 h, then diluted with 20 mL of water and 5 mL of 1M HCl. The precipitate was filtered, washed with additional water, then taken up in 15 mL of methanol and 5 mL of DMSO while still moist. To the mixture was added 1 mL of 25% w/v NaOH_{(aq.)}, and the solution was stirred at ambient temperature for 30 min. The methanol was removed in vacuo, and the remaining solution was diluted with water (25 mL) and 1 M HCl (15 mL). The suspension was extracted with ethyl acetate (2×15 mL), then the combined organic layers were back extracted with water (1×10 mL), and brine (1×10 mL), dried over MgSO₄, filtered, and concentrated to a solid. Trituration with hot acetonitrile gave 63 mg (28%) of a pale yellow solid. (MS, M⁺+H=497).

### Preparation of 6-(3-aminopropoxy)-2-phenyl-N-(2-(thiazolo[5,4-b]pyridin-2-yl)phenyl)pyrimidine-4-carboxamide hydrochloride

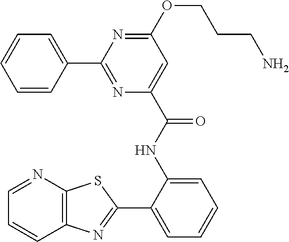

To a solution of 75 mg (1.0 mmol) of 4-amino-1-butanol in 1 mL of DMSO was added 40 mg (1.0 mmol) of 60% NaH in mineral oil. The suspension was stirred and heated briefly to disperse the NaH, then cooled back to ambient temperature. Next, 100 mg (0.226 mmol) of 6-chloro-2-phenyl-N-(2-(thiazolo[5,4-b]pyridin-2-yl)phenyl)pyrimidine-4-carboxamide was added. The reaction was stirred at ambient temperature for 30 min, and the initial suspension cleared to give an orange solution within a few minutes. The reaction was diluted with 10 mL of water and 4 mL of pentane. The mixture was stirred, and the pentane was removed to extract mineral oil from the product, then the precipitate was filtered, washed with water, and suspended again in water. To this suspension was added 1 mL of 1M HCl, then the precipitate was filtered and dried on the filter to give 40 mg (34%) of the product as a light yellow solid. (MS, M⁺+H=483).

### Preparation of 6-(2-(3-methyl-1H-pyrazol-1-yl)ethylamino)-2-phenyl-N-(2-(thiazolo[5,4-b]pyridin-2-yl)phenyl)pyrimidine-4-carboxamide

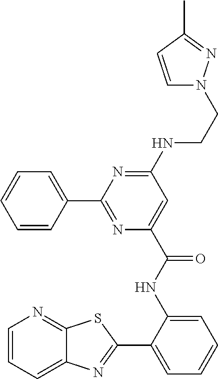

Prepared by general method A, using 6-chloro-2-phenyl-N-(2-(thiazolo[5,4-b]pyridin-2-yl)phenyl)pyrimidine-4-carboxamide and 2-(3-methyl-1H-pyrazol-1-yl)ethylamine. 97% yield, (MS, M⁺+H=533).

### Preparation of 6-chloro-2-phenyl-N-(2-(thiazolo[5,4-c]pyridin-2-yl)phenyl)pyrimidine-4-carboxamide

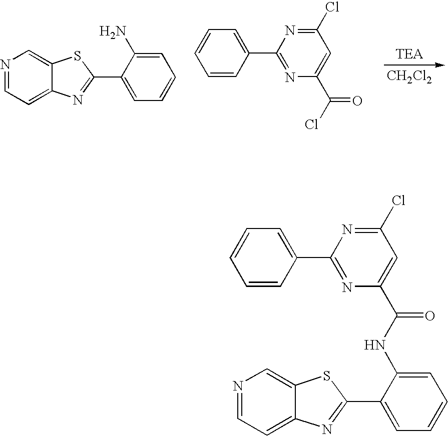

A solution of 6-chloro-2-phenylpyrimidine-4-carbonyl chloride (14 g, 55.3 mmol) in CH₂Cl₂ (100 mL) was added to a solution of 2-(thiazolo[5,4-c]pyridine-2-yl)aniline (7.5 g, 32 mmol) in CH₂Cl₂ (400 mL), and triethylamine (50 mL, 359 mmol). The reaction mixture was stirred at ambient temperature. The product began to crystallize within a few minutes. After stirring 16 hours, the reaction was concentrated to remove the solvent. The residue was diluted with MeOH (500 mL). The solid was collected by filtration, washed with additional MeOH and dried under vacuum to give 6-chloro-2-phenyl-N-(2-(thiazolo[5,4-c]pyridin-2-yl)phenyl)pyrimidine-4-carboxamide (11.2 g, 77% yield). (MS, M⁺+H=444).

### General Method B

### Preparation of 6-(4-(2-methoxyethyl)piperazin-1-yl)-2-phenyl-N-(2-(thiazolo[5,4-c]pyridin-2-yl)phenyl)pyrimidine-4-carboxamide hydrochloride

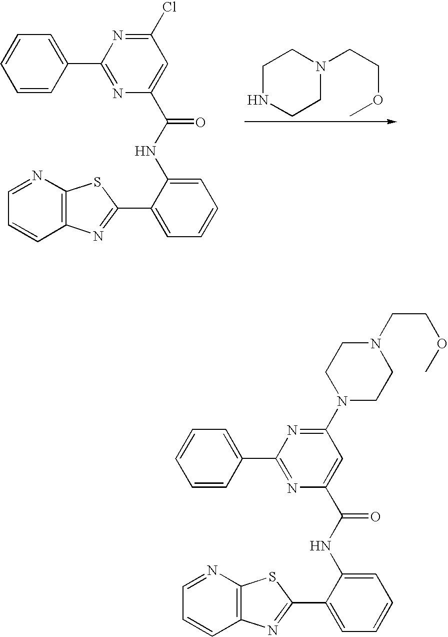

2-(methoxyethyl)piperazine (1.6 g, 11.3 mmol) was added to a suspension 6-chloro-2-phenyl-N-(2-(thiazolo[5,4-c]pyridin-2-yl)phenyl)pyrimidine-4-carboxamide (500 mg, 1.13 mmol) in THF (10 mL). The reaction mixture was heated at reflux for 2 hours becoming homogeneous upon heating. After cooling to room temperature, the reaction mixture was diluted with H₂O (20 mL) and the resulting solid was collected by filtration, rinsed with H₂O, then CH₃CN and dried under vacuum to give 6-(4-(2-methoxyethyl)piperazin-1-yl)-2-phenyl-N-(2-(thiazolo[5,4-c]pyridin-2-yl)phenyl)pyrimidine-4-carboxamide (592 mg, 95% yield). The solid was dissolved in CH₂Cl₂ (15 mL). HCl/MeOH (1.25 M, 3.2 mmol) was added and the mixture stirred for 30 min. The solid was collected by filtration, rinsed with CH₂Cl₂, then Et₂O and dried under vacuum to give the HCl salt. (MS, M⁺+H=552).

### Preparation of 6-morpholino-2-phenyl-N-(2-(thiazolo[5,4-c]pyridin-2-yl)phenyl)pyrimidine-4-carboxamide

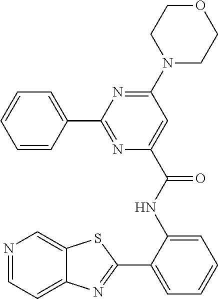

General method B was used employing 6-chloro-2-phenyl-N-(2-(thiazolo[5,4-c]pyridin-2-yl)phenyl)pyrimidine-4-carboxamide and morpholine. 85% yield, (MS, M⁺+H=495).

### Preparation of 2-phenyl-6-(piperazin-1-yl)-N-(2-(thiazolo[5,4-c]pyridin-2-yl)phenyl)pyrimidine-4-carboxamide hydrochloride

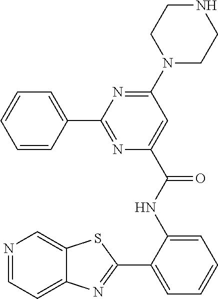

General method B was used employing 6-chloro-2-phenyl-N-(2-(thiazolo[5,4-c]pyridin-2-yl)phenyl)pyrimidine-4-carboxamide and tert-butyl piperazinecarboxylate followed by treatment with 20% TFA in DCM. The solvent was evaporated, the residue was taken up in acetonitrile/water 1:2.5, 1N aqueous HCl (20 equiv) was added and the resulting solution was lyophilized to afford quantitative yield of the title compound. 85% yield, (MS, M⁺+H=494).

### Preparation of 6-(4-isopropylpiperazin-1-yl)-2-phenyl-N-(2-(thiazolo[5,4-c]pyridin-2-yl)phenyl)pyrimidine-4-carboxamide hydrochloride

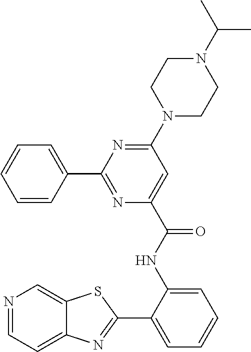

General method B was used employing 6-chloro-2-phenyl-N-(2-(thiazolo[5,4-c]pyridin-2-yl)phenyl)pyrimidine-4-carboxamide and 1-isopropylpiperazine. 72% yield, (MS, M⁺+H=536).

### Preparation of 6-(2-methoxyethylamino)-2-phenyl-N-(2-(thiazolo[5,4-c]pyridin-2-yl)phenyl)pyrimidine-4-carboxamide

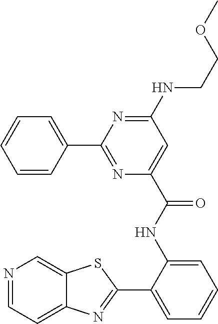

General method B was used employing 6-chloro-2-phenyl-N-(2-(thiazolo[5,4-c]pyridin-2-yl)phenyl)pyrimidine-4-carboxamide and 2-methoxyethylamine. 79% yield, (MS, M⁺+H=483).

### Preparation of 6-(2-methoxyethyl)(methyl)amino)-2-phenyl-N-(2-(thiazolo[5,4-c]pyridin-2-yl)phenyl)pyrimidine-4-carboxamide

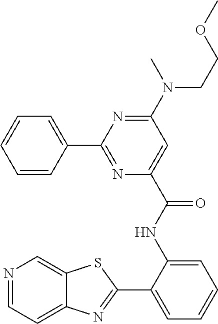

General method B was used employing 6-chloro-2-phenyl-N-(2-(thiazolo[5,4-c]pyridin-2-yl)phenyl)pyrimidine-4-carboxamide and N-(2-methoxyethyl)methylamine. 97% yield, (MS, M⁺+H=497).

### Preparation of 6-(2-morpholinoethylamino)-2-phenyl-N-(2-(thiazolo[5,4-c]pyridin-2-yl)phenyl)pyrimidine-4-carboxamide hydrochloride

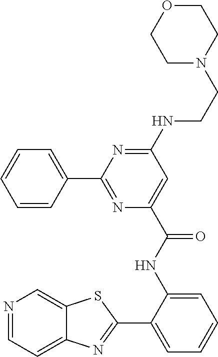

General method B was used employing 6-chloro-2-phenyl-N-(2-(thiazolo[5,4-c]pyridin-2-yl)phenyl)pyrimidine-4-carboxamide and 2-morpholinoethylamine. 76% yield, (MS, M⁺+H=538).

### Preparation of 6-(pyridin-3-ylmethylamino)-2-phenyl-N-(2-(thiazolo[5,4-c]pyridin-2-yl)phenyl)pyrimidine-4-carboxamide hydrochloride

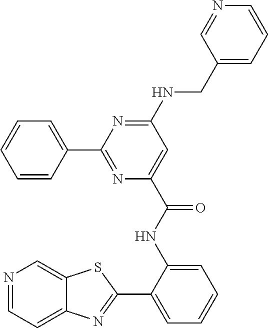

General method B was used employing 6-chloro-2-phenyl-N-(2-(thiazolo[5,4-c]pyridin-2-yl)phenyl)pyrimidine-4-carboxamide and 3-(aminomethyl)pyridine. 65% yield, (MS, M⁺+H=516).

### Preparation of 6-(pyridin-4-ylmethylamino)-2-phenyl-N-(2-(thiazolo[5,4-c]pyridin-2-yl)phenyl)pyrimidine-4-carboxamide hydrochloride

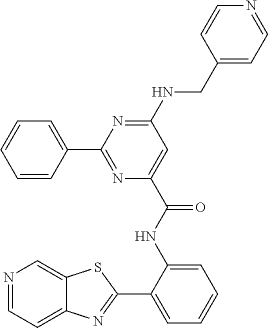

General method B was used employing 6-chloro-2-phenyl-N-(2-(thiazolo[5,4-c]pyridin-2-yl)phenyl)pyrimidine-4-carboxamide and 4-(aminomethyl)pyridine. 40% yield, (MS, M⁺+H=516).

### Preparation of 6-(2-pyrrolidin-1-yl)ethylamino)-2-phenyl-N-(2-(thiazolo[5,4-c]pyridin-2-yl)phenyl)pyrimidine-4-carboxamide hydrochloride

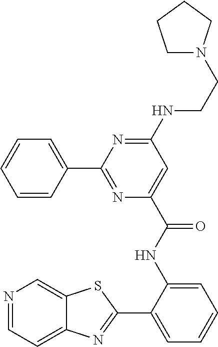

General method B was used employing 6-chloro-2-phenyl-N-(2-(thiazolo[5,4-c]pyridin-2-yl)phenyl)pyrimidine-4-carboxamide and 2-(pyrrolidin-1-yl)-ethylamine. 34% yield, (MS, M⁺+H=522).

### Preparation of 6-(pyridin-2-ylmethylamino)-2-phenyl-N-(2-(thiazolo[5,4-c]pyridin-2-yl)phenyl)pyrimidine-4-carboxamide hydrochloride

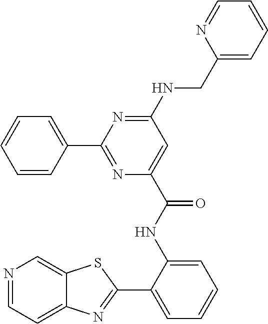

General method B was used employing 6-chloro-2-phenyl-N-(2-(thiazolo[5,4-c]pyridin-2-yl)phenyl)pyrimidine-4-carboxamide and 2-(aminomethyl)pyridine. 76% yield, (MS, M⁺+H=516).

### Preparation of methyl 5-formyl-6-phenylpicolinate

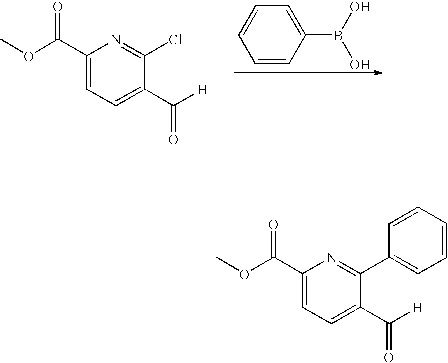

Methyl 6-chloro-5-formylpicolinate (1.0 grams, 5.0 mmol), phenylboronic acid (670 mg, 1.1 equiv), Pd(dppf):CH₂Cl₂ (180 mg, 0.05 equiv), and KF (430 mg, 1.5 equiv) were dissolved in DMF (15 mL, nitrogen flushed) in a microwave tube. The reaction was heated in the microwave (140° C.×10 min.), exposed to air for 2 hours, filtered, and concentrated. The residue was purified by silica gel chromatography (0 to 100% gradient of EtOAc in pentane.) The product fractions were concentrated to dryness, purified a second time by silica gel chromatography, and concentrated to dryness. The residue was triturated in ether:pentane and filtered to obtain 732 mg (61% yield) of methyl 5-formyl-6-phenylpicolinate as a white solid. A second crop (195 mg, 16%) was obtained from concentrating the mother liquor. (MS, M⁺+H=242).

Methyl 6-chloro-5-formylpicolinate was prepared as described in Gangadasu, B.; Narender, P.; Kumar, S. Bharath; Ravinder, M.; Rao, B. Ananda; Ramesh, Ch.; Raju, B. China; Rao, V. Jayathirtha “Facile and selective synthesis of chloronicotinaldehydes by the Vilsmeier reaction,” Tetrahedron 2006, 62, 8398.

### Preparation of methyl 5-(morpholinomethyl)-6-phenylpicolinate

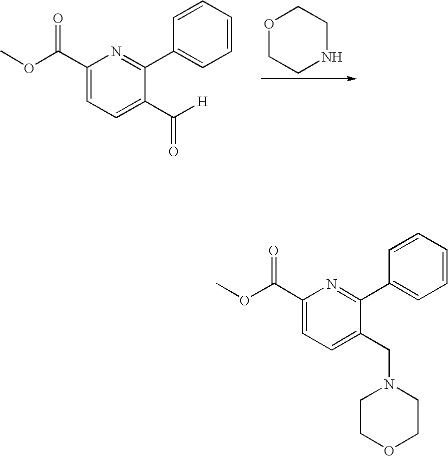

Methyl 5-formyl-6-phenylpicolinate (241 mg, 1.0 mmol), and morpholine (131 μL, 1.5 equiv) were stirred in anhydrous CH₂Cl₂ (4 mL) for 2 hours and then concentrated to dryness. The residue was dissolved in THF, and stirred with Na(OAc)₃BH (254 mg) for 3 hours. Methanol was then added and the reaction was stirred for an additional 8 hours. An additional charge of Na(OAc)₃BH (254 mg) was added and the reaction was stirred for 4 hours and then quenched with methanol and water. The mixture was concentrated, suspended in CH₂Cl₂/aqueous NaHCO₃ (satd), and the organic layer was concentrated to dryness. The crude product was purified by silica gel chromatography (0 to 100% gradient of EtOAc in pentane.) The fractions were concentrated to dryness and chased with pentane to obtain methyl 5-(morpholinomethyl)-6-phenylpicolinate as a tacky solid. (155 mg, 50% yield). (MS, M⁺+H=313).

### Preparation of 5-(morpholinomethyl)-6-phenyl-N-(2-(thiazolo[5,4-b]pyridin-2-yl)phenyl)picolinamide

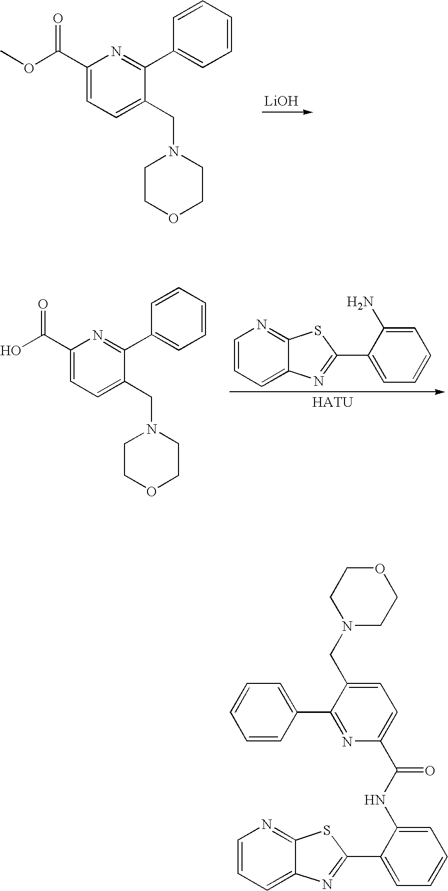

Methyl 5-(morpholinomethyl)-6-phenylpicolinate (155 mg, 0.5 mmol), and LiOH (58 mg, 5 equiv) were stirred in 1:1 THF:water for 18 hours. The reaction mixture was concentrated to dryness, dissolved in water, made acidic with 4N HCl (1 mL) and lyophilized. The residue was dissolved in DMF (4 mL). One half of the DMF solution (0.25 mmol) was stirred with diisopropylethylamine (DIEA) (0.260 mL, 1.5 equiv) and 2-(7-Aza-1H-benzotriazole-1-yl)-1,1,3,3-tetramethyluronium hexafluorophosphate (HATU) (143 mg, 6 equiv) for 10 min at room temperature. 2-(Thiazolo[5,4-b]pyridin-2-yl)aniline (57 mg, 1 equiv) was added and the reaction mixture was stirred for 60 hours at 40° C. The reaction mixture was quenched with water. The solids were collected by filtration, and triturated in hot methanol. The HCl salt was prepared by the addition of HCl in methanol and concentration to dryness to obtain 5-(morpholinomethyl)-6-phenyl-N-(2-(thiazolo[5,4-b]pyridin-2-yl)phenyl)picolinamide as the HCl salt (12 mg, 9% yield) (MS, M⁺+H=508).

### Preparation of 4-(1-tert-Butoxycarbonyl-azetidin-3-yloxy)-6-phenyl-pyridine-2-carboxylic acid

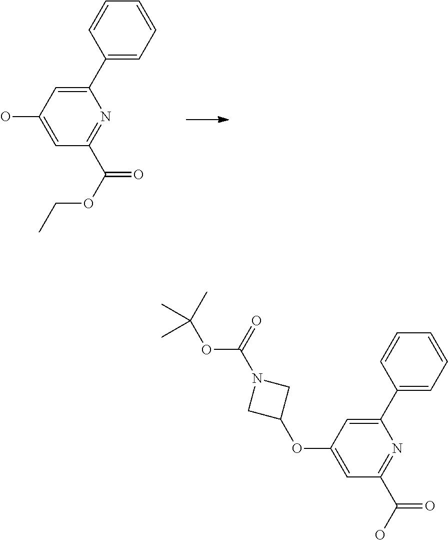

To a stirred solution of 4-hydroxy-6-phenylpyridine-2-carboxylic acid (243 mg, 1.0 mmol) in tetrahydrofuran (THF) (5 mL) was added 1-Boc-3-hydroxyazetidine (217 mg, 1.25 mmol), followed by triphenylphosphine (328 mg, 1.25 mmol) and dropwise addition of diisopropylazodicarboxylate (DIAD) (0.25 mL, 1.25 mmol). The resulting solution was heated at 55° C. for 12 h. The solvent was evaporated and the residue was diluted with 1N NaHSO₄ solution, and extracted with chloroform. The organic phase was washed with diluted NaHCO₃ solution, dried over sodium sulfate, filtered and concentrated. The residue was purified by silica gel chromatography eluting with a 0-50% gradient of ethyl acetate in pentane. Obtained 292 mg of product, contaminated by an equimolar amount of diisopropyl hydrazine-1,2-dicarboxylate.

This intermediate was treated with LiOH (172 mg, 7.2 mmol) in THF (4 mL) and methanol (2 mL) for 1 h. The mixture was acidified with 1N aqueous HCl solution to pH=3, and extracted with ethyl acetate. The organic extracts were washed with brine, dried over sodium sulfate, filtered and concentrated to afford the title compound as an oil, still contaminated by diisopropyl hydrazine-1,2-dicarboxylate.

### Preparation of 4-(Azetidin-3-yloxy)-6-phenyl-pyridine-2-carboxylic acid (2-thiazolo[5,4-b]pyridin-2-yl-phenyl)-amide

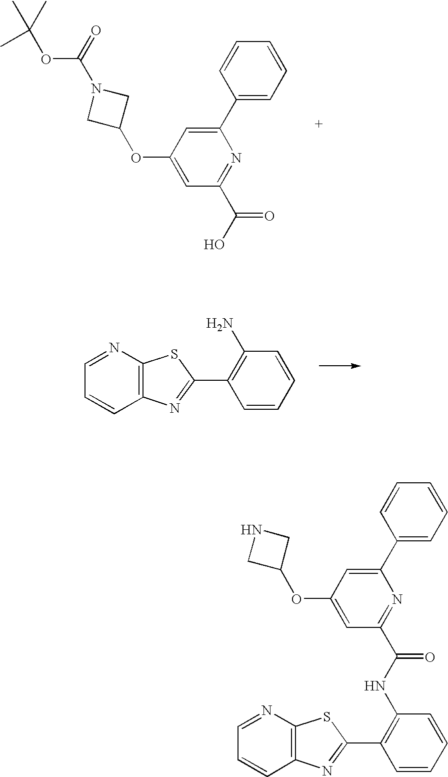

A mixture of 4-(1-tert-Butoxycarbonyl-azetidin-3-yloxy)-6-phenyl-pyridine-2-carboxylic acid (178 mg, 0.48 mmol), 2-thiazolo[5,4-b]pyridin-2-yl-phenylamine (114 mg, 0.5 mmol), HATU (219 mg, 0.58 mmol), DIEA (0.167 mL, 0.96 mmol) in DMF (56 mL) was stirred at room temperature overnight. The reaction mixture was diluted with water (15 mL). The solid was collected by filtration, washed with water and air dried. The crude product was triturated in a mixture of acetonitrile/ethyl acetate 3:1 (5 mL) and water (1 mL), filtered and air dried to afford 162 mg (58%) of the Boc protected title compound.

This intermediate was treated with 20% TFA in dichloromethane (5 mL) for 1 h. The solvent was evaporated and the residue was taken up in 2 mL of acetonitrile. Water was added (10 mL) and the mixture was neutralized with 1N NaOH until the product precipitated. It was collected by filtration and washed with water. This solid was suspended in acetonitrile (3 mL) and water (3 mL) and triturated at 40° C. for 15 min. After cooling to room temperature, it was collected by filtration and air dried to afford 103 mg (78%) of the title compound as the free base. The free base was suspended in acetonitrile and water 1:4 (10 mL), 1N aqueous HCl was added (0.5 mL), more acetonitrile was added until homogeneous. Excess acetonitrile was evaporated and the resulting cloudy solution was lyophilized to afford 116 mg (74%) of the title compound as the HCl salt. (MS, M⁺+H=480).

### Preparation of 4-Hydroxy-6-phenyl-pyridine-2-carboxylic acid (2-thiazolo[5,4-b]pyridin-2-yl-phenyl)-amide

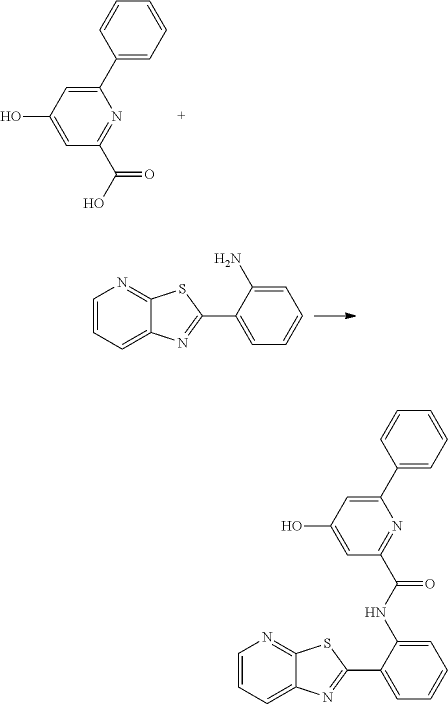

4-Hydroxy-6-phenylpyridine-2-carboxylic acid (2.77 g, 12.83 mmol) was mixed with 2-thiazolo[5,4-b]pyridin-2-yl-phenylamine (2.62 g, 11.66 mmol), HATU (5.75 g, 15.16 mmol), and DIEA (6 mL, 35 mmol) in DMF (130 mL). The resulting suspension was heated at 45° C. overnight. The reaction mixture was cooled to room temperature and water was added (100 mL). The resulting solid was collected by filtration, washed with water and air dried. The crude product was suspended in 200 mL of 1:1 acetonitrile/ethyl acetate and stirred at 40° C. for 1 h. After cooling to room temperature the solid was collected by filtration and air dried. 3.5 g of the title compound were obtained as a yellow solid in 85% purity (55% yield, adjusted for purity). A small portion (96 mg) was further purified by trituration with hot ethyl acetate and filtration to afford 76 mg of 4-Hydroxy-6-phenyl-pyridine-2-carboxylic acid (2-thiazolo[5,4-b]pyridin-2-yl-phenyl)-amide (MS, M⁺+H=425).

### Preparation of 4-(2-Bromo-ethoxy)-6-phenyl-pyridine-2-carboxylic acid (2-thiazolo[5,4-h]pyridin-2-yl-phenyl)-amide

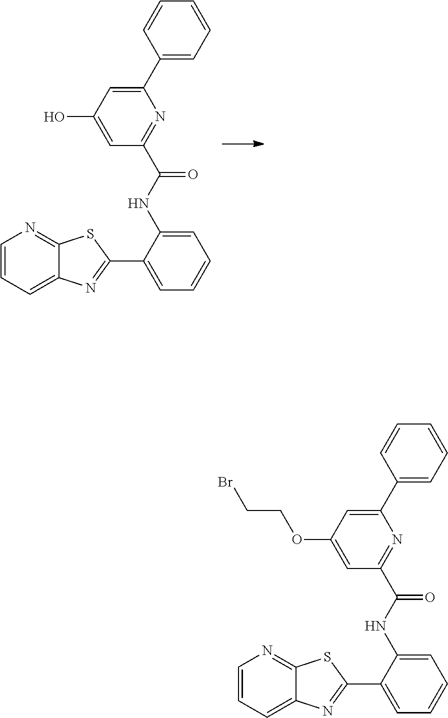

4-Hydroxy-6-phenyl-pyridine-2-carboxylic acid (2-thiazolo[5,4-b]pyridin-2-yl-phenyl)-amide (1.0 g, 2.0 mmol) was dissolved in DMSO (60 mL) at 80° C. The solution was cooled to room temperature and added dropwise to a stirred mixture of potassium carbonate (691 mg, 5.0 mmol) and dibromoethane (3.45 mL, 40.0 mmol) at 70° C. After addition was complete, the reaction mixture was stirred at 80° C. for 2 h. After cooling to room temperature, the mixture was diluted with water and extracted with dichloromethane (3×50 mL). The combined organic extracts were washed with brine (2×30 mL) dried over sodium sulfate, filtered and concentrated. The residue was purified by silica gel chromatography, eluting with a 20-50% gradient of ethyl acetate in pentane. Obtained 495 mg (46%) of the title compound as a brown solid.

### General Method C

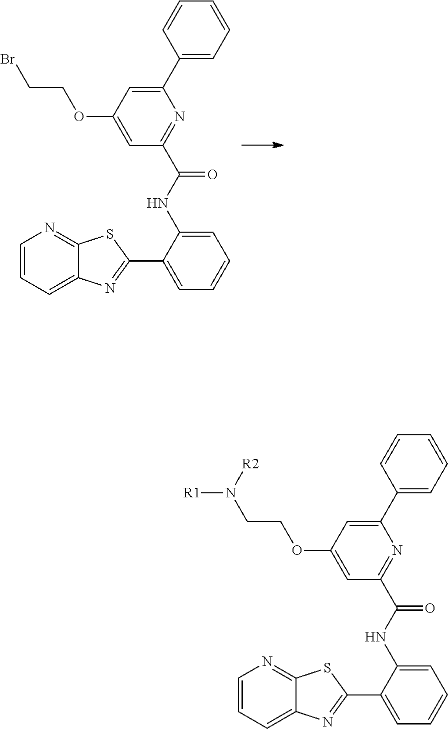

4-(2-Bromo-ethoxy)-6-phenyl-pyridine-2-carboxylic acid (2-thiazolo[5,4-b]pyridin-2-yl-phenyl)-amide (250 mg, 0.47 mmol) and the appropriate amine (4-10 equiv) were mixed in THF (10 mL) in a sealed tube and heated to 100° C. until the reaction was complete, typically 12-24 h. After cooling to room temperature, the reaction mixture was diluted with 10 mL of water. The product precipitated and was filtered and washed with acetonitrile. The crude product was further purified, if needed, by trituration with acetonitrile or by preparative high performance liquid chromatography (HPLC).

### Preparation 4-[2-(1,1-Dioxo-1-thiomorpholin-4-yl)-ethoxy]-6-phenyl-pyridine-2-carboxylic acid (2-thiazolo[5,4-b]pyridin-2-yl-phenyl)-amide hydrochloride

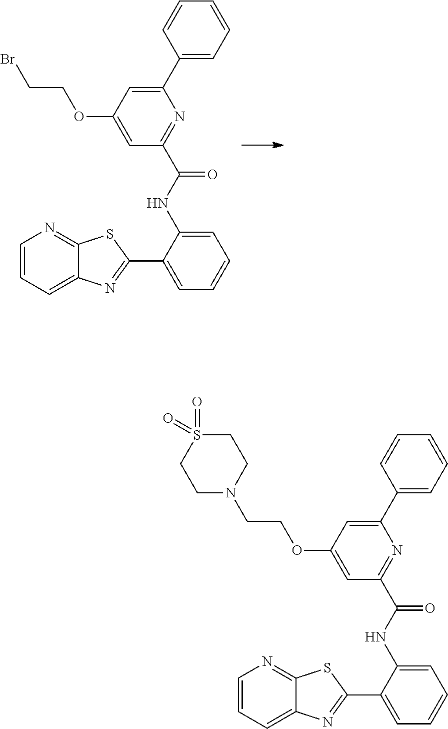

The title compound was prepared according to general method C by reacting thiomorpholine dioxide (8 equiv) with 4-(2-Bromo-ethoxy)-6-phenyl-pyridine-2-carboxylic acid (2-thiazolo[5,4-b]pyridin-2-yl-phenyl)-amide, and was obtained in 93% yield. The product was suspended in acetonitrile/water 1:4 and 1N aqueous HCl solution was added (3 equiv). The resulting slurry was sonicated then lyophilized to afford the title compound as the HCl salt (MS, M⁺+H=586).

### Preparation of 4-{2-[4-(2-Methoxy-ethyl)-piperazin-1-yl]-ethoxy}-6-phenyl-pyridine-2-carboxylic acid (2-thiazolo[5,4-b]pyridin-2-yl-phenyl)-amide hydrochloride

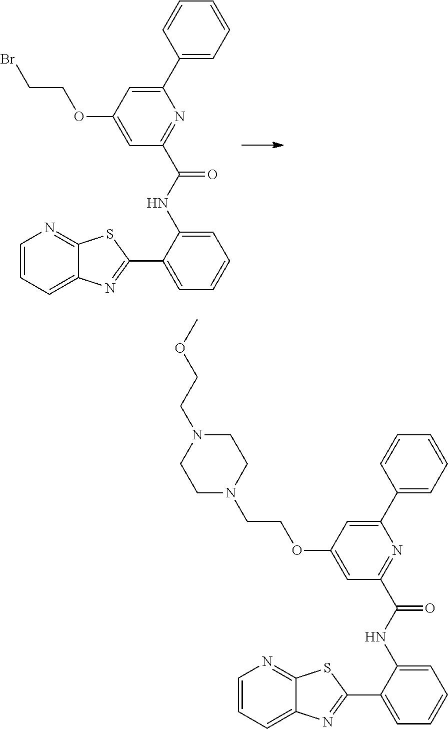

The title compound was prepared according to general method C by reacting 1-(2-methoxyethyl)piperazine (8 equiv) with 4-(2-Bromo-ethoxy)-6-phenyl-pyridine-2-carboxylic acid (2-thiazolo[5,4-b]pyridin-2-yl-phenyl)-amide, and was obtained in 83% yield. The product was suspended in acetonitrile/water 1:4 and 1 N aqueous HCl solution was added (3 equiv). The resulting slurry was sonicated then lyophilized to afford the title compound as the HCl salt (MS, M⁺+H=595).

### Preparation of 4-[2-(2-Dimethylamino-ethylamino)-ethoxy]-6-phenyl-pyridine-2-carboxylic acid (2-thiazolo[5,4-b]pyridin-2-yl-phenyl)-amide hydrochloride

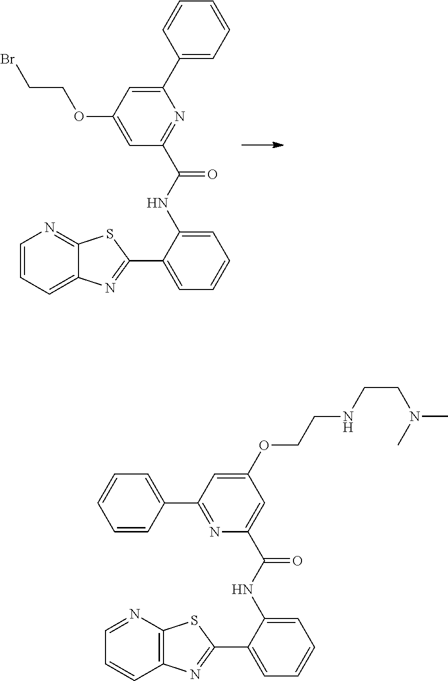

The title compound was prepared according to general method C by reacting N,N-diethylenediamine (8 equiv) with 4-(2-Bromo-ethoxy)-6-phenyl-pyridine-2-carboxylic acid (2-thiazolo[5,4-b]pyridin-2-yl-phenyl)-amide, and was purified by preparative HPLC. The fractions from HPLC were concentrated, then treated with 1 N aqueous HCl solution and lyophilized to afford 46 mg (38%) of the title compound as the HCl salt (MS, M⁺+H=539).

### Preparation of 4-[2-((2R,6S)-2,6-Dimethyl-morpholin-4-yl)-ethoxy]-6-phenyl-pyridine-2-carboxylic acid (2-thiazolo[5,4-b]pyridin-2-yl-phenyl)-amide

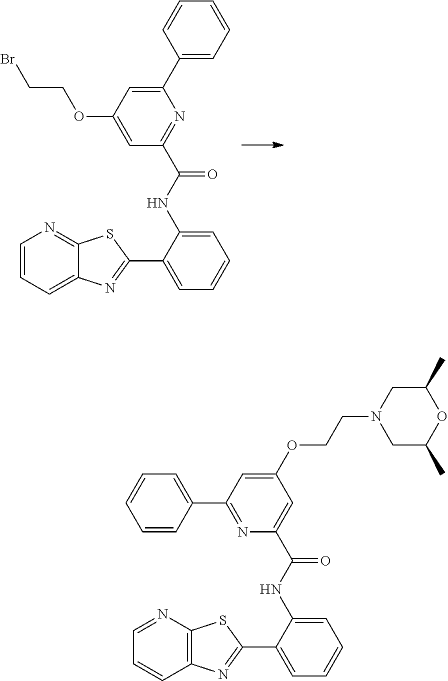

The title compound was prepared according to general method C by reacting cis-2,6-dimethylmorpholine (10 equiv) with 4-(2-Bromo-ethoxy)-6-phenyl-pyridine-2-carboxylic acid (2-thiazolo[5,4-b]pyridin-2-yl-phenyl)-amide. The crude product was purified by trituration with acetonitrile (3 mL). The title compound was obtained in 71% yield as the free base (MS, M⁺+H=566).

### Preparation of 4-{2-[(2-Methoxy-ethyl)-methyl-amino]-ethoxy}-6-phenyl-pyridine-2-carboxylic acid (2-thiazolo[5,4-b]pyridin-2-yl-phenyl)-amide

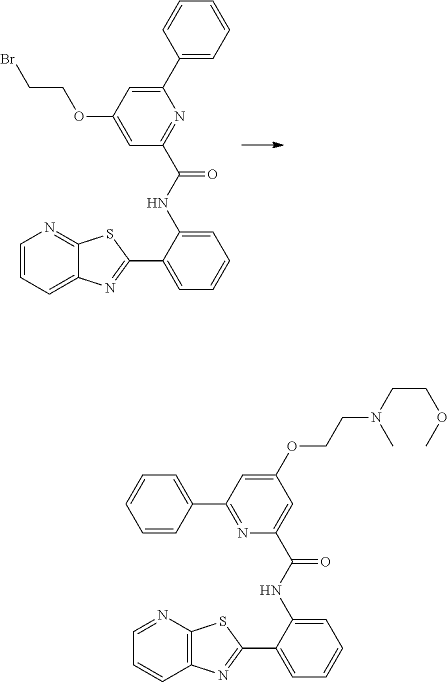

The title compound was prepared according to general method C by reacting N-(2-methoxyethyl)methylamine (4 equiv) with 4-(2-Bromo-ethoxy)-6-phenyl-pyridine-2-carboxylic acid (2-thiazolo[5,4-b]pyridin-2-yl-phenyl)-amide. The crude product was purified by repeated trituration with acetonitrile and ethyl acetate. The title compound was obtained in 2% yield as the free base (MS, M⁺+H=540).

### Preparation of 4-(2-Bromo-ethoxy)-6-phenyl-pyridine-2-carboxylic acid ethyl ester

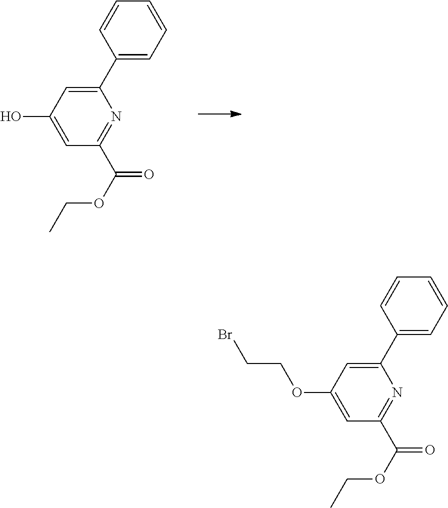

To a stirred suspension of potassium carbonate (829 mg, 6.0 mmol) in dibromoethane (3.5 mL, 41 mmol) at 80° C. was added a solution of 4-Hydroxy-6-phenyl-pyridine-2-carboxylic acid ethyl ester (973 mg, 4.0 mmol) in acetonitrile (30 mL) over 2 h. The mixture was cooled to room temperature and stirred for 2 h. The solvent was removed in vacuo. The residue was diluted with water and extracted with ethyl acetate (3×30 mL). The combined organic extracts were washed with brine, dried over sodium sulfate, and concentrated. The residue was purified by silica gel chromatography, eluting with a 10-50% gradient of ethyl acetate in pentane to afford 1.01 g (72%) of the title compound as a white solid.

### Preparation of 4-(2-Morpholin-4-yl-ethoxy)-6-phenyl-pyridine-2-carboxylic acid ethyl ester

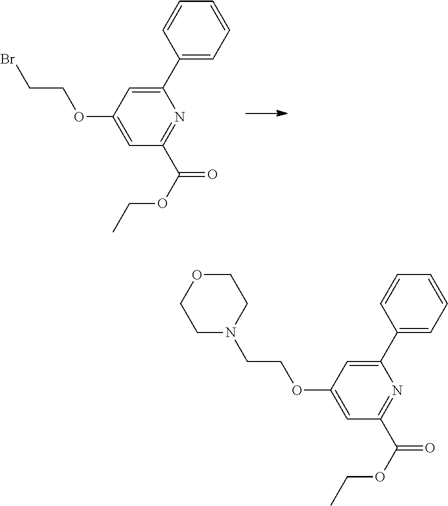

A solution of 4-(2-Bromo-ethoxy)-6-phenyl-pyridine-2-carboxylic acid ethyl ester (350 mg, 1.0 mmol) and morpholine (0.435 mL, 5.0 mmol) in acetonitrile (5 mL) was stirred at 60° C. for 2 h. The solvent was evaporated and the residue was diluted with water and extracted with ethyl acetate (3×20 mL). The combined organic extracts were dried over sodium sulfate and concentrated to afford 362 mg (100%) of the title compound.

### Preparation of 4-(2-Morpholin-4-yl-ethoxy)-6-phenyl-pyridine-2-carboxylic acid

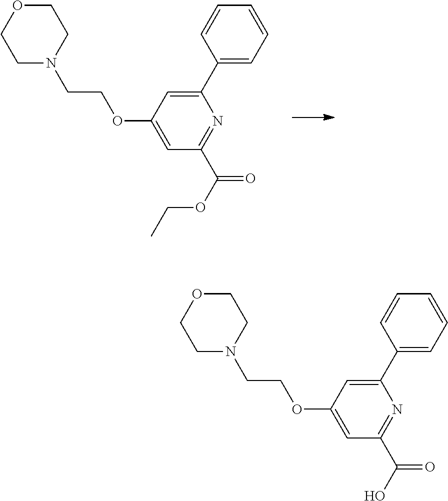

4-(2-Morpholin-4-yl-ethoxy)-6-phenyl-pyridine-2-carboxylic acid ethyl ester (356 mg, 1.0 mmol) was treated with lithium hydroxide (120 mg, 5.0 mmol) in a mixture of THF (4 mL) and methanol (2 mL) for 1 h. The solvent was evaporated and the residue was diluted with water and acidified with 1N aqueous HCl solution to pH=3. The resulting aqueous solution was lyophilized to give 671 mg of the title compound as a mixture with LiCl.

### Preparation of 4-(2-Morpholin-4-yl-ethoxy)-6-phenyl-pyridine-2-carboxylic acid (2-thiazolo[5,4-b]pyridin-2-yl-phenyl)-amide hydrochloride

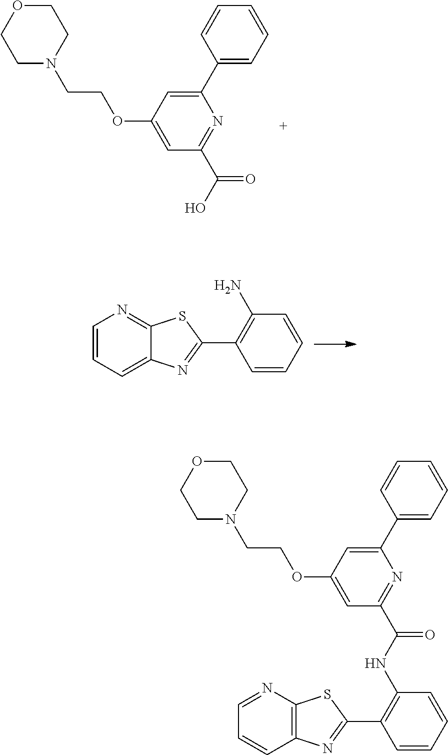

A mixture of 4-(2-Morpholin-4-yl-ethoxy)-6-phenyl-pyridine-2-carboxylic acid as a mixture with LiCl (540 mg), 2-thiazolo[5,4-b]pyridin-2-yl-phenylamine (182 mg, 0.8 mmol), HATU (456 mg, 1.2 mmol), DIEA (0.28 mL, 1.6 mmol) in DMF (10 mL) was stirred at 40° C. overnight, then at room temperature for 48 h. The reaction mixture was diluted with water (10 mL). The solid that formed was collected by filtration, washed with water and air dried. The crude product was suspended in acetonitrile/water 1:4 (120 mL) and 1N aqueous HCl solution was added (3 equiv). The resulting suspension was sonicated and then lyophilized to afford 344 mg (74%) of the title compound as the HCl salt. (MS, M⁺+H=538).

### Preparation of 4-[2-(2-Ethoxycarbonyl-6-phenyl-pyridin-4-yloxy)-ethyl]-piperazine-1-carboxylic acid tert-butyl ester

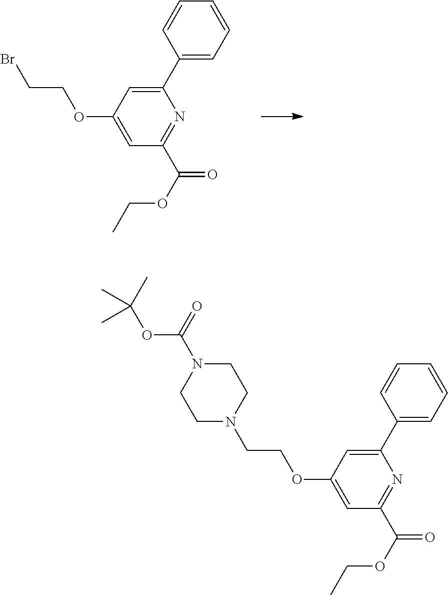

A solution of 4-(2-Bromo-ethoxy)-6-phenyl-pyridine-2-carboxylic acid ethyl ester (350 mg, 1.0 mmol), N-Boc piperazine (279 mg, 1.5 mmol), and DIEA (0.35 mL, 2.0 mmol) in acetonitrile (5 mL) was stirred at 60° C. overnight. The solvent was evaporated and the residue was diluted with water and extracted with ethyl acetate (3×20 mL). The combined organic extracts were dried over sodium sulfate, and concentrated to afford 502 mg (100%) of the title compound.

### Preparation of 4-[2-(2-Carboxy-6-phenyl-pyridin-4-yloxy)-ethyl]-piperazine-1-carboxylic acid tert-butyl ester

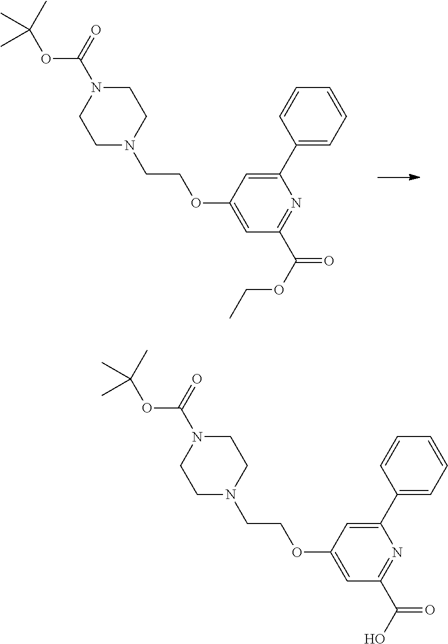

4-[2-(2-Ethoxycarbonyl-6-phenyl-pyridin-4-yloxy)-ethyl]-piperazine-1-carboxylic acid tert-butyl ester (455 mg, 1.0 mmol) was treated with lithium hydroxide (120 mg, 5.0 mmol) in a mixture of THF (4 mL) and methanol (2 mL) for 1 h. The solvent was evaporated and the residue was diluted with water and acidified with 1N aqueous HCl solution to pH=3. Upon standing the product precipitated and was collected by filtration and air dried to give 170 mg (40%) of the title compound.

### Preparation of 6-Phenyl-4-(2-piperazin-1-yl-ethoxy)-pyridine-2-carboxylic acid (2-thiazolo[5,4-b]pyridin-2-yl-phenyl)-amide hydrochloride

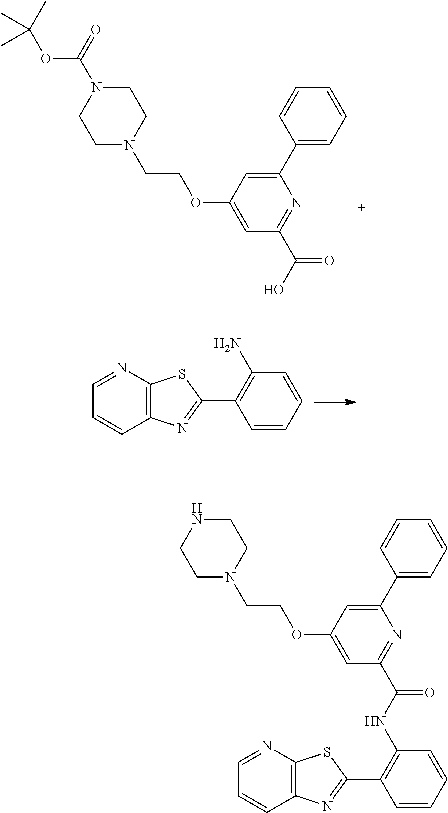

A mixture of 4-[2-(2-Carboxy-6-phenyl-pyridin-4-yloxy)-ethyl]-piperazine-1-carboxylic acid tert-butyl ester (170 mg, 0.4 mmol), 2-thiazolo[5,4-b]pyridin-2-yl-phenylamine (78 mg, 0.32 mmol), HATU (182 mg, 0.48 mmol), DIEA (0.14 mL, 0.8 mmol) in DMF (4 mL) was stirred at 40° C. for 36 h. The reaction mixture was diluted with water (10 mL). The solid that precipitated was filtered, washed with water and air dried to afford 133 mg (65%) of product. The crude product was treated with 20% TFA in dichloromethane (4 mL) for 1 h. The solvent was evaporated and the residue diluted with 2 mL acetonitrile and 3 mL of water. 1N aqueous HCl solution was added (10 equiv) and the resulting suspension was sonicated and lyophilized to afford 138 mg of the title compound as the HCl salt. (MS, M⁺+H=537).

### Preparation of 6-(3-Hydroxy-prop-1-ynyl)-2-phenyl-pyrimidine-4-carboxylic acid (2-thiazolo[5,4-b]pyridin-2-yl-phenyl)-amide

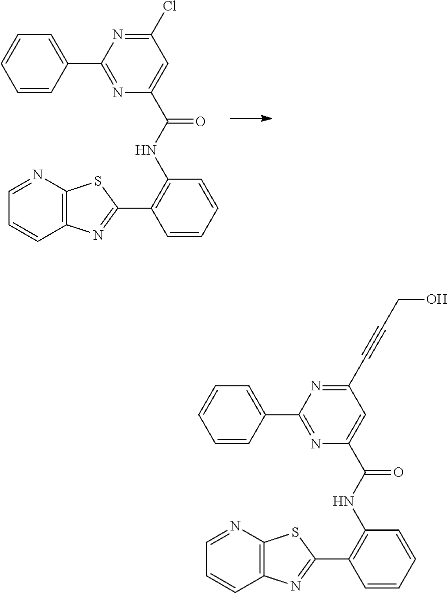

Nitrogen was bubbled through a mixture of 6-Chloro-2-phenyl-pyrimidine-4-carboxylic acid (2-thiazolo[5,4-b]pyridin-2-yl-phenyl)-amide (177 mg, 0.4 mmol), propargyl alcohol (59 μL, 1.0 mmol), Cl₂Pd(PPh₃)₂ (18 mg, 0.025 mmol), CuI (7.5 mg, 0.04 mmol), and triethylamine (0.35 mL, 2.5 mmol) in THF (4 mL) in a microwave tube for 5 minutes. The tube was capped and the mixture was heated in a microwave oven at 100° C. for 30 min. The reaction mixture was diluted with water and brine and extracted with chloroform (3×20 mL). The combined organic extracts were washed with brine, dried over sodium sulfate, and concentrated. The residue was purified by silica gel chromatography, eluting with a 0-80% gradient of ethyl acetate in pentane to afford 102 mg (55%) of slightly impure product. This product was triturated in a mixture of methanol (3 mL), acetonitrile (3 mL) and ethyl acetate (2 mL) at 50° C. for 10 min, then collected by filtration and air dried to afford 53 mg of pure title compound (MS, M⁺+H=464).

### Preparation of 2-Chloro-6-morpholin-4-yl-pyrimidine-4-carboxylic acid methyl ester

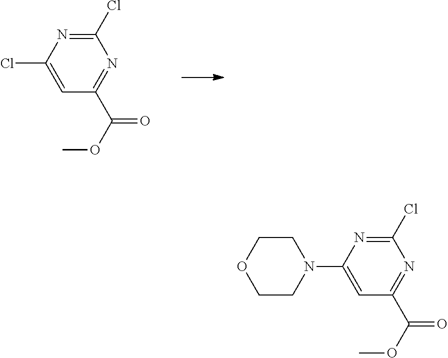

To a solution of 2,6-dichloro-pyrimidine-4-carboxylic acid methyl ester (1.035 g, 5.0 mmol) in DCM (12 mL) at 0° C. was added triethylamine (0.7 mL, 5.0 mmol), followed by a solution of morpholine (463 mg, 5.0 mmol) in DCM (8 mL). The resulting solution was stirred at 0° C. for 30 min. The reaction mixture was diluted with water and extracted with ethyl acetate (3×80 mL). The combined organic extracts were dried over sodium sulfate, filtered and concentrated to afford 1.215 g (94%) of the title compound as a white solid.

### Preparation of 6-Morpholin-4-yl-2-phenyl-pyrimidine-4-carboxylic acid

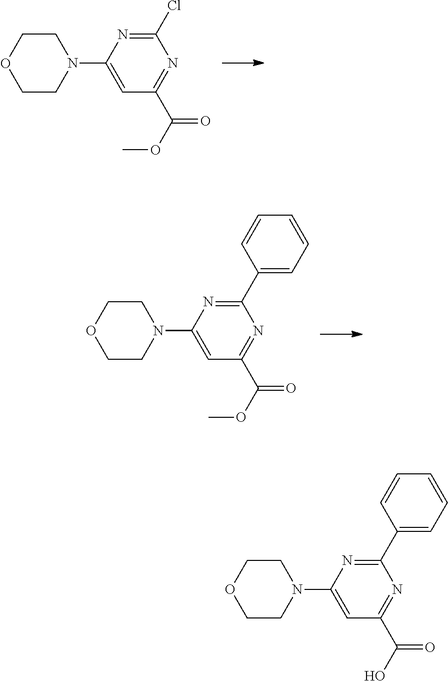

A microwave tube was charged with 2-Chloro-6-morpholin-4-yl-pyrimidine-4-carboxylic acid methyl ester (515 mg, 2.0 mmol), phenylboronic acid (390 mg, 3.2 mmol) and Pd(PPh₃)₄ (185 mg, 0.16 mmol). Acetonitrile was added (39 mL), and nitrogen was bubbled through the solution for 5 min. Triethylamine (558 μL, 4.0 mmol) was added and the resulting mixture was heated in a microwave oven at 160° C. for 2 h. The reaction mixture was diluted with water and extracted with ethyl acetate (3×30 mL). The combined organic extracts were dried over sodium sulfate, filtered and concentrated to dryness. The residue was purified by silica gel chromatography, eluting with a 0-50% gradient of ethyl acetate in pentane. Obtained 240 mg (40%) of 6-morpholin-4-yl-2-phenyl-pyrimidine-4-carboxylic acid methyl ester as a white solid.

This intermediate (227 mg, 1.32 mmol) was treated with LiOH (158 mg, 6.59 mmol) in THF (5 mL) and MeOH (4 mL) for 1 h. Water was added, the mixture was acidified with 1N aqueous HCl solution to pH=2 and extracted with ethyl acetate (2×30 mL). The combined organic extracts were dried over sodium sulfate, and concentrated to afford 220 mg (58%) of the title compound as a white solid.

### Preparation of 6-Morpholin-4-yl-2-phenyl-pyrimidine-4-carboxylic acid (2-thiazolo[5,4-b]pyridin-2-yl-phenyl)-amide

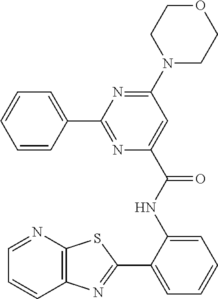

The title compound was prepared in 24% yield according to the procedure outlined for 4-(2-Morpholin-4-yl-ethoxy)-6-phenyl-pyridine-2-carboxylic acid (2-thiazolo[5,4-b]pyridin-2-yl-phenyl)-amide, by reacting 6-Morpholin-4-yl-2-phenyl-pyrimidine-4-carboxylic acid with 2-thiazolo[5,4-b]pyridin-2-yl-phenylamine. It was purified by trituration in hot acetonitrile/methanol 1:1 (MS, M⁺+H=495).

### Preparation of methyl 6-methyl-2-phenylpyrimidine-4-carboxylate

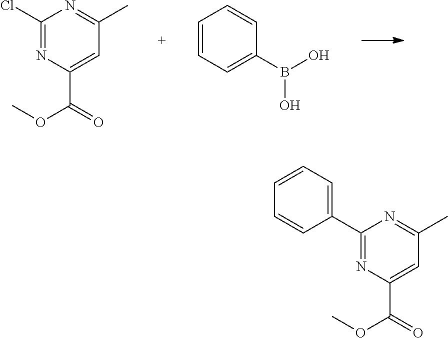

To a mixture of methyl 2-chloro-6-methylpyrimidine-4-carboxylate (1.87 g, 10 mmol), phenylboronic acid (1.34 g, 11 mmol), Pd₂(dba)₃ (0.14 g, 0.15 mmol) and tri-tert-butylphosphine (4 mL, 18 mmol, 10% wt in hexane) in THF (50 mL) at room temperature was added KF (1.9 g, 3.3 mmol) and the reaction mixture was heated at reflux temperature for 8 h. TLC monitored (petroleum ether:ethyl acetate=5:1). After cooling to room temperature, the mixture was filtered through a pad of celite and the filtrate was concentrated in vacuo. Purification of the crude product by medium pressure liquid chromatography on silica gel (with eluent of petroleum ether:ethyl acetate=20:1 to 10:1) gave methyl 6-methyl-2-phenylpyrimidine-4-carboxylate as a white solid (1.0 g, 44%).

### Preparation of methyl 6-(bromomethyl)-2-phenylpyrimidine-4-carboxylate

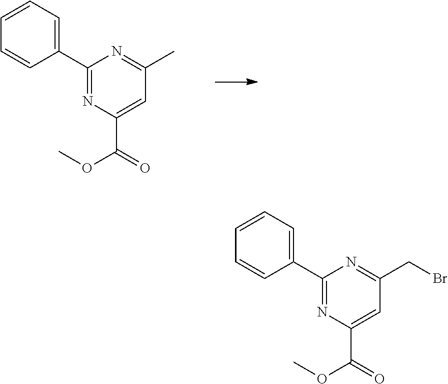

A mixture of methyl 6-methyl-2-phenylpyrimidine-4-carboxylate (0.5 g, 2.2 mmol) and Br₂ (0.35 g, 2.2 mmol) in 10 ml of acetic acid was warmed to 80° C. for 1 h. The mixture was concentrated under reduced pressure. Purification of the crude product by medium pressure liquid chromatography on silica gel (with eluent of petroleum ether:ethyl acetate=50:1 to 40:1) gave methyl 6-(bromomethyl)-2-phenylpyrimidine-4-carboxylate as a brown solid. (39 mg, 6%).

### Preparation of methyl 2-phenyl-6-(pyrrolidin-1-ylmethyl)pyrimidine-4-carboxylate

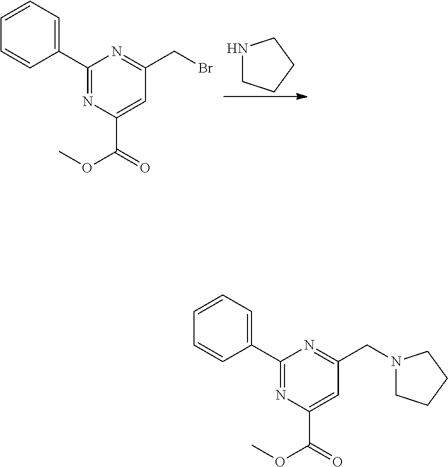

Methyl 6-(bromomethyl)-2-phenylpyrimidine-4-carboxylate (1.2 g, 3.9 mmol) was dissolved in DCM (30 mL), DIEA (1.3 mL, 7.8 mmol) and pyrrolidine (1.3 mL, 7.8 mmol) were added. The resulting mixture was stirred at room temperature overnight. The mixture was concentrated under reduced pressure. The residue was purified by silica column chromatography (with eluent of petroleum ether:ethyl acetate=10:1 to 5:1) to give methyl 2-phenyl-6-(pyrrolidin-1-ylmethyl)pyrimidine-4-carboxylate as a white solid. (0.8 g, 69%).

### Preparation of 2-phenyl-6-(pyrrolidin-1-ylmethyl)pyrimidine-4-carboxylic acid

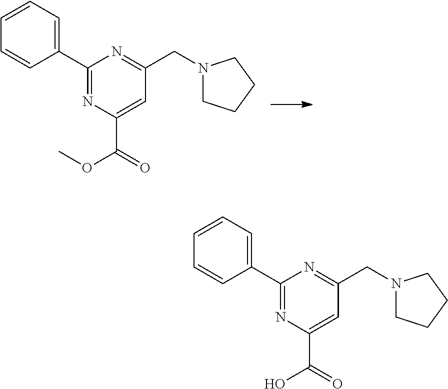

Methyl 2-phenyl-6-(pyrrolidin-1-ylmethyl)pyrimidine-4-carboxylate (1.12 g, 3.8 mmol), LiOH.H₂O (0.47 g, 11.3 mmol) THF (10 mL), CH₃OH (10 mL) and H₂O (5 mL) were added into a flask. The resulting mixture was stirred at room temperature for 4 h. The reaction mixture was diluted with H₂O (20 mL) and acidified by 1N HCl to pH=5. The white precipitate was collected by filtration to give 2-phenyl-6-(pyrrolidin-1-ylmethyl)pyrimidine-4-carboxylic acid as a white solid. (0.92 g, 86%).

### Preparation of 2-Phenyl-6-pyrrolidin-1-ylmethyl-pyrimidine-4-carboxylic acid (2-thiazolo[5,4-b]pyridin-2-yl-phenyl)-amide hydrochloride

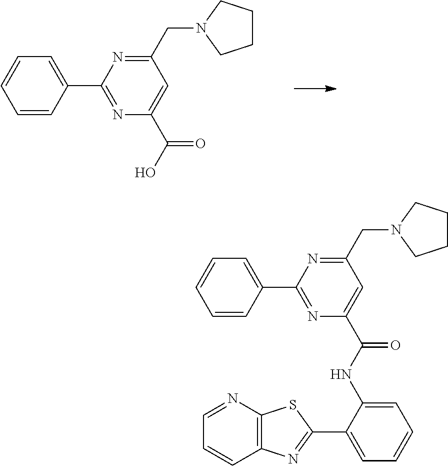

The title compound was prepared according to procedure outlined for 4-(2-Morpholin-4-yl-ethoxy)-6-phenyl-pyridine-2-carboxylic acid (2-thiazolo[5,4-b]pyridin-2-yl-phenyl)-amide, by reacting 2-Phenyl-6-pyrrolidin-1-ylmethyl-pyrimidine-4-carboxylic acid with 2-thiazolo[5,4-b]pyridin-2-yl-phenylamine. It was purified by trituration with hot acetonitrile. The free base was suspended in acetonitrile/water 1:1, treated with 1N aqueous HCl solution (1.5 equiv) and lyophilized to afford the title compound in 65% yield (MS, M⁺+H=493).

### Preparation of compound methyl 6-(morpholinomethyl)-2-phenylpyrimidine-4-carboxylate

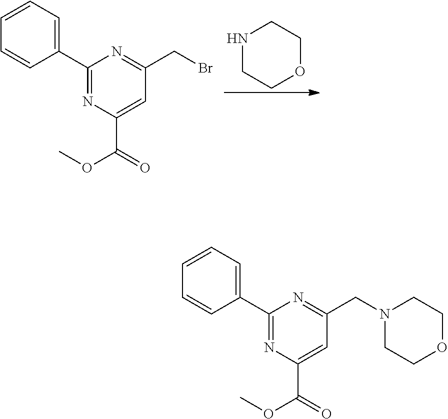

Methyl 6-(bromomethyl)-2-phenylpyrimidine-4-carboxylate (0.83 g, 2.7 mmol) was dissolved in DCM (13 mL), DIEA (0.9 mL, 5.4 mmol) and morpholine (0.28 mL, 3.2 mmol) were added. The resulting mixture was stirred at room temperature overnight. The mixture was concentrated, the residue was purified by silica column chromatography (with eluent of petroleum ether:ethyl acetate=10:1 to 5:1) to give methyl 6-(morpholinomethyl)-2-phenylpyrimidine-4-carboxylate as a white solid (0.78 g, 92%).

### Preparation of compound 6-(morpholinomethyl)-2-phenylpyrimidine-4-carboxylic acid

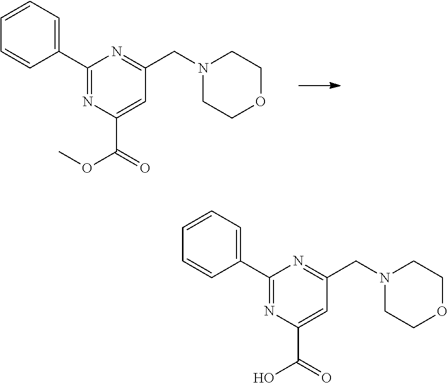

Methyl 6-(morpholinomethyl)-2-phenylpyrimidine-4-carboxylate (1.6 g, 5.3 mmol), LiOH.H₂O (0.67 g, 15.9 mmol), THF (10 mL), CH₃OH (10 mL) and H₂O (5 mL) were added into a flask. The resulting mixture was stirred at room temperature for 4 h. The reaction mixture was diluted by H₂O (20 mL) and acidified by 1N HCl to pH=5. The white precipitate was collected to give 6-(morpholinomethyl)-2-phenylpyrimidine-4-carboxylic acid as a white solid. (0.7 g, 46%).

### Preparation of 6-Morpholin-4-ylmethyl-2-phenyl-pyrimidine-4-carboxylic acid (2-thiazolo[5,4-b]pyridin-2-yl-phenyl)-amide hydrochloride

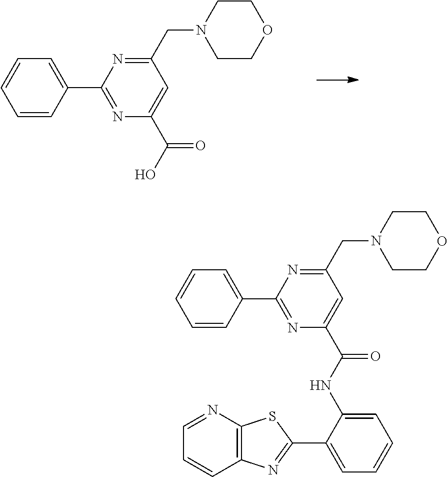

The title compound was prepared according to procedure outlined for 4-(2-Morpholin-4-yl-ethoxy)-6-phenyl-pyridine-2-carboxylic acid (2-thiazolo[5,4-b]pyridin-2-yl-phenyl)-amide, by reacting 6-Morpholin-4-ylmethyl-2-phenyl-pyrimidine-4-carboxylic acid with 2-thiazolo[5,4-b]pyridin-2-yl-phenylamine. It was purified by preparative HPLC. The fractions from HPLC were evaporated, treated with 1N aqueous HCl solution and lyophilized to afford the title compound in 14% yield (MS, M⁺+H=509).

### Preparation of 2-Chloro-6-[4-(2-methoxy-ethyl)-piperazin-1-yl]-pyrimidine-4-carboxylic acid methyl ester

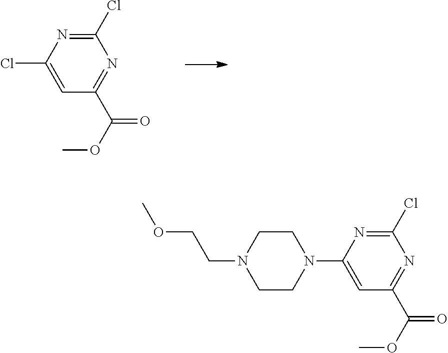

To a solution of 2,6-dichloro-pyrimidine-4-carboxylic acid methyl ester (580 mg, 2.8 mmol) in DCM (6 mL) at 0° C. was added triethylamine (0.39 mL, 2.8 mmol), followed by a solution of 1-(2-methoxyethyl)piperazine (406 mg, 2.8 mmol) in DCM (6 mL). The resulting solution was stirred at 0° C. for 30 min. The reaction mixture was diluted with water and extracted with DCM (3×80 mL). The combined organic extracts were dried over sodium sulfate, filtered and concentrated to afford 870 mg (99%) of the title compound as a pale yellow solid.

### Preparation of 6-[4-(2-Methoxy-ethyl)-piperazin-1-yl]-2-phenyl-pyrimidine-4-carboxylic acid

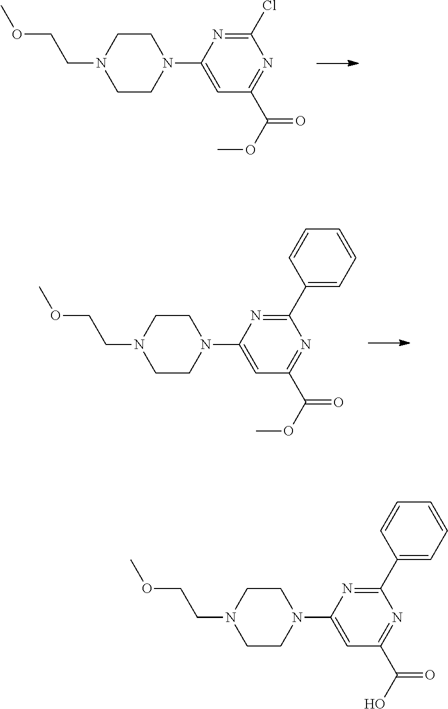

A microwave tube was charged with 2-Chloro-6-[4-(2-methoxy-ethyl)-piperazin-1-yl]-pyrimidine-4-carboxylic acid methyl ester (630 mg, 2.0 mmol), phenylboronic acid (390 mg, 3.2 mmol) and Pd(PPh₃)₄ (185 mg, 0.16 mmol). Acetonitrile was added (38 mL), and nitrogen was bubbled through the solution for 5 min. Triethylamine (558 μL, 4.0 mmol) was added and the resulting mixture was heated in a microwave oven at 160° C. for 2 h. The reaction mixture was diluted with water and extracted with ethyl acetate (3×40 mL). The combined organic extracts were washed twice with brine and once with water, dried over sodium sulfate, filtered and concentrated. The residue was partially purified by silica gel chromatography, eluting with a 0-4% gradient of MeOH in DCM. Obtained 640 mg (90%) of 6-[4-(2-Methoxy-ethyl)-piperazin-1-yl]-2-phenyl-pyrimidine-4-carboxylic acid methyl ester as an orange oil contaminated with 2-Chloro-6-[4-(2-methoxy-ethyl)-piperazin-1-yl]-pyrimidine-4-carboxylic acid methyl ester. The crude product was treated with LiOH (215 mg, 8.9 mmol, 5 equiv) in THF (10 mL) and MeOH (10 mL) for 1 h. Water was added, the mixture was neutralized with 1N aqueous HCl solution and lyophilized to afford 988 mg of the title compound as the adduct with LiCl contaminated with 2-Chloro-6-[4-(2-methoxy-ethyl)-piperazin-1-yl]-pyrimidine-4-carboxylic acid and 2-Methoxy-6-[4-(2-methoxy-ethyl)-piperazin-1-yl]-pyrimidine-4-carboxylic acid.

### Preparation of 2-Chloro-6-[4-(2-methoxy-ethyl)-piperazin-1-yl]-pyrimidine-4-carboxylic acid (2-thiazolo[5,4-b]pyridin-2-yl-phenyl)-amide hydrochloride

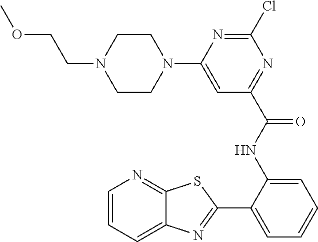

The title compound was prepared according to the procedure outlined for 4-(2-Morpholin-4-yl-ethoxy)-6-phenyl-pyridine-2-carboxylic acid (2-thiazolo[5,4-b]pyridin-2-yl-phenyl)-amide by reacting the crude product from above with 2-thiazolo[5,4-b]pyridin-2-yl-phenylamine. It was isolated by preparative HPLC as a by-product of the reaction. The fractions from HPLC were evaporated, treated with 1N aqueous HCl and lyophilized to afford the title compound in 8% yield (MS, M⁺+H=510)

### Preparation of 2-Methoxy-6-[4-(2-methoxy-ethyl)-piperazin-1-yl]-pyrimidine-4-carboxylic acid (2-thiazolo[5,4-b]pyridin-2-yl-phenyl)-amide hydrochloride

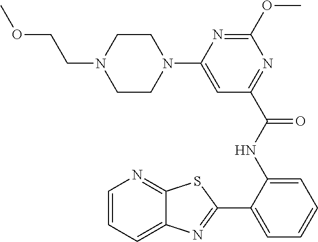

The title compound was prepared according to the procedure outlined for 4-(2-Morpholin-4-yl-ethoxy)-6-phenyl-pyridine-2-carboxylic acid (2-thiazolo[5,4-b]pyridin-2-yl-phenyl)-amide by reacting the crude product from above with 2-thiazolo[5,4-b]pyridin-2-yl-phenylamine. It was isolated by preparative HPLC as a by-product of the reaction. The fractions from HPLC were evaporated, treated with 1N aqueous HCl and lyophilized to afford the title compound in 6% yield (MS, M⁺+H=506).

### Preparation of 6-hydroxypyrimidine-4-carboxylic acid

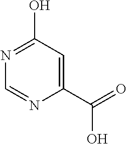

To a suspension of 2.52 g (12.0 mmol) of diethyloxaloacetate sodium salt in 8 mL of water was added 1.9 mL of 6.25 M NaOH_{(aq.)}, dropwise over 1 min. The mixture was stirred at ambient temperature for 40 min to give an orange solution. Next, 2.1 g (26 mmol) of formamidine hydrochloride in 2 mL of water was added. The reaction was cooled with an ice bath, and with the aid of a pH meter, the pH was maintained between 11 and 11.5, by the addition of 6.25 M NaOH as the reaction progressed over 40 min. The pH was then adjusted to 1 by the addition of 12 M HCl, giving a white precipitate. This was filtered, washed with 0.1 M HCl (2×5 mL), then dried on the filter to give 618 mg (37%) of a light tan solid.

### Preparation of 6-chloropyrimidine-4-carbonyl chloride

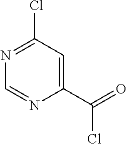

To 500 mg (3.57 mmol) of 6-hydroxypyrimidine-4-carboxylic acid was added 1 mL of POCl₃. The reaction was heated at reflux for 30 min, during which time a black mass formed. The POCl₃ was removed in vacuo, then the residue was scraped from the sides of the flask and stirred with 20 mL of pentane. The pentane slurry was extracted with water (2×5 mL), and brine (1×5 mL), draining any black insoluble material with the aqueous layers, dried over MgSO₄, filtered, and concentrated to an oil. Cooling with a dry-ice acetone bath induced crystallization, giving 250 mg (40%) of the acid chloride as a pale yellow crystalline solid.

### Preparation of 6-chloro-N-(2-(thiazolo[5,4-b]pyridin-2-yl)phenyl)pyrimidine-4-carboxamide

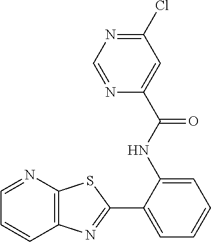

To a solution of 62 mg (0.271 mmol) of 2-(thiazolo[5,4-b]pyridin-2-yl)aniline in 2 mL of chloroform was added 100 μL (0.574 mmol) of Hunig's base. To this was added a solution of 48 mg (0.27 mmol) of 6-chloropyrimidine-4-carbonyl chloride in 1 mL of chloroform at ambient temperature. A precipitate formed rapidly. The mixture was stirred for 10 min, then 15 mL of methanol was added. The precipitate was filtered, washed with additional methanol, and dried on the filter to give 62 mg (62%) of a pale yellow solid.

### Preparation of 6-(2-(dimethylamino)ethylamino)-N-(2-(thiazolo[5,4-b]pyridin-2-yl)phenyl)pyrimidine-4-carboxamide

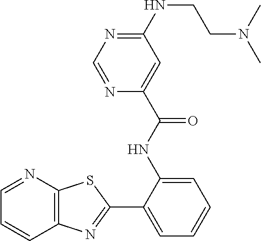

To 50 mg of 6-chloro-N-(2-(thiazolo[5,4-b]pyridin-2-yl)phenyl)pyrimidine-4-carboxamide suspended in 1.5 mL of THF was added 50 μL (0.46 mmol) of N,N-dimethylethylamine. The mixture was heated at reflux for 30 min, then diluted with 10 mL of water. The precipitate was filtered, washed with water, and dried on the filter to give 30 mg (55%) of a white solid. (MS, M⁺+H=420).

### Preparation of 6-hydroxy-2-methylpyrimidine-4-carboxylic acid

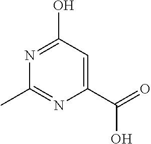

Prepared according to the procedure for 6-hydroxy-2-phenylpyrimidine-4-carboxylic acid, substituting acetamidine hydrochloride for benzamidine hydrochloride. Yield 930 mg (60%) of a light tan solid.

### Preparation of 6-chloro-2-methylpyrimidine-4-carbonyl chloride

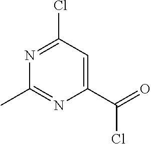

To 920 mg (5.97 mmol) of 6-hydroxy-2-methylpyrimidine-4-carboxylic acid was added 13 mL of POCl₃. The mixture was heated at reflux for 1 h, then concentrated in vacuo to a brown oil. This was suspended in 25 mL of pentane and extracted with water (2×10 mL). The combined water layers were back extracted with pentane (1×25 mL), then the combined pentane layers were washed again with water (2×10 mL), dried over MgSO₄, filtered, and concentrated to 494 mg (43%) of a colorless oil.

### Preparation of 6-chloro-2-methyl-N-(2-(thiazolo[5,4-b]pyridin-2-yl)phenyl)pyrimidine-4-carboxamide

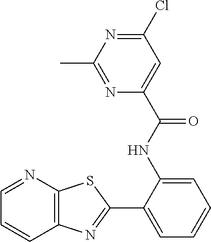

Prepared according to the procedure for 6-chloro-N-(2-(thiazolo[5,4-b]pyridin-2-yl)phenyl)pyrimidine-4-carboxamide, substituting 6-chloro-2-methylpyrimidine-4-carbonyl chloride for 6-chloro-2-methylpyrimidine-4-carbonyl chloride. Yield 602 mg (61%) of a yellow solid.

### Preparation of 6-(2-(dimethylamino)ethylamino)-2-methyl-N-(2-(thiazolo[5,4-b]pyridin-2-yl)phenyl)pyrimidine-4-carboxamide

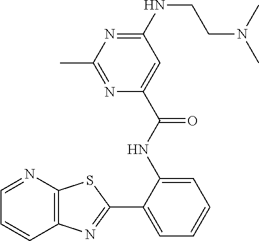

Prepared according to the procedure for 6-(2-(dimethylamino)ethylamino)-N-(2-(thiazolo[5,4-b]pyridin-2-yl)phenyl)pyrimidine-4-carboxamide, substituting 6-(2-(dimethylamino)ethylamino)-2-methyl-N-(2-(thiazolo[5,4-b]pyridin-2-yl)phenyl)pyrimidine-4-carboxamide for 6-(2-(dimethylamino)ethylamino)-N-(2-(thiazolo[5,4-b]pyridin-2-yl)phenyl)pyrimidine-4-carboxamide. The product was recrystallized from ethyl acetate to give 32 mg (56%) of a white solid. (MS, M⁺+H=434).

### Example 2

### Biological Activity

A mass spectrometry based assay was used to identify modulators of SIRT1 activity. The mass spectrometry based assay utilizes a peptide having 20 amino acid residues as follows: Ac-EE-K(biotin)-GQSTSSHSK(Ac)NleSTEG-K(5TMR)-EE-NH2 (SEQ ID NO: 1) wherein K(Ac) is an acetylated lysine residue and Nle is a norleucine. The peptide is labeled with the fluorophore 5TMR (excitation 540 nm/emission 580 nm) at the C-terminus. The sequence of the peptide substrate is based on p53 with several modifications. In addition, the methionine residue naturally present in the sequence was replaced with the norleucine because the methionine may be susceptible to oxidation during synthesis and purification.

The mass spectrometry assay is conducted as follows: 0.5 μM peptide substrate and 120 μM βNAD⁺ is incubated with 10 nM SIRT1 for 25 minutes at 25° C. in a reaction buffer (50 mM Tris-acetate pH 8, 137 mM NaCl, 2.7 mM KCl, 1 mM MgCl₂, 5 mM DTT, 0.05% BSA). Test compounds may be added to the reaction as described above. The SirT1 gene is cloned into a T7-promoter containing vector and transformed into BL21(DE3). After the 25 minute incubation with SIRT1, 10 μL of 10% formic acid is added to stop the reaction. Reactions are sealed and frozen for later mass spec analysis. Determination of the mass of the substrate peptide allows for precise determination of the degree of acetylation (i.e. starting material) as compared to deacetylated peptide (product).

A control for inhibition of sirtuin activity is conducted by adding 1 μL of 500 mM nicotinamide as a negative control at the start of the reaction (e.g., permits determination of maximum sirtuin inhibition). A control for activation of sirtuin activity is conducted using 10 nM of sirtuin protein, with 1 μL of DMSO in place of compound, to determine the amount of deacetylation of the substrate at a given timepoint within the linear range of the assay. This timepoint is the same as that used for test compounds and, within the linear range, the endpoint represents a change in velocity.

For the above assay, SIRT1 protein was expressed and purified as follows. The SirT1 gene was cloned into a T7-promoter containing vector and transformed into BL21(DE3). The protein was expressed by induction with 1 mM IPTG as an N-terminal His-tag fusion protein at 18° C. overnight and harvested at 30,000×g. Cells were lysed with lysozyme in lysis buffer (50 mM Tris-HCl, 2 mM Tris[2-carboxyethyl]phosphine (TCEP), 10 μM ZnCl₂, 200 mM NaCl) and further treated with sonication for 10 min for complete lysis. The protein was purified over a Ni-NTA column (Amersham) and fractions containing pure protein were pooled, concentrated and run over a sizing column (Sephadex S200 26/60 global). The peak containing soluble protein was collected and run on an Ion-exchange column (MonoQ). Gradient elution (200 mM-500 mM NaCl) yielded pure protein. This protein was concentrated and dialyzed against dialysis buffer (20 mM Tris-HCl, 2 mM TCEP) overnight. The protein was aliquoted and frozen at −80° C. until further use.

Sirtuin modulating compounds that activated SIRT1 were identified using the assay described above and are shown below in Table 1. The EC₁₋₅ values for the activating compounds are represented by A′ (EC_{1.5}<250 nM), A (EC_{1.5}≧250 nM and ≦1 uM), B (EC_{1.5}>1 and ≦10 uM), or C (EC_{1.5}>10 uM). The percent maximum fold activation is represented by A (Fold activation≧300%), B (Fold Activation≧150% and <300%), or C (Fold Activation<150%).

TABLE 1

| COMPOUND | | | | FOLD |
|---|---|---|---|---|
| NO. | [M + H]+ | STRUCTURE | EC_{1.5} | ACT. |
| 1 | 494 | 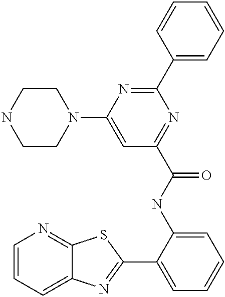 | A′ | A |
| 2 | 495 | 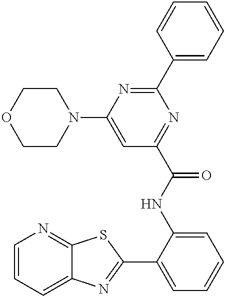 | A′ | B |
| 3 | 552 | 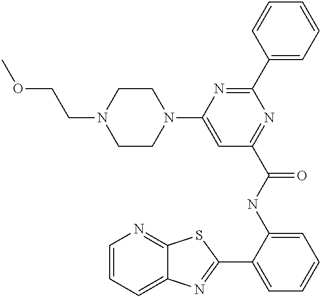 | A′ | A |
| 4 | 510 | 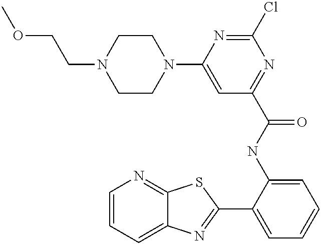 | A | A |
| 5 | 506 | 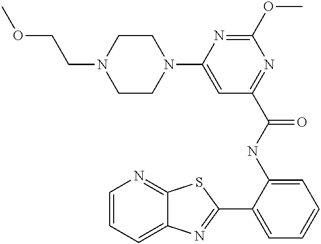 | A | A |
| 6 | 480 | 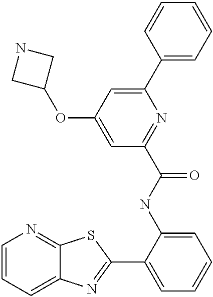 | A | A |
| 7 | 586 | 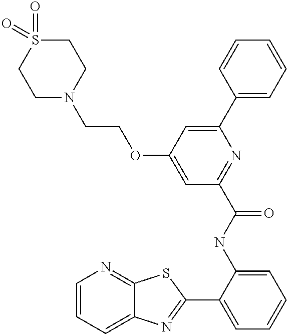 | A′ | A |
| 8 | 444 | 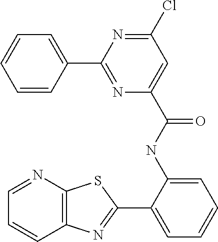 | C | C |
| 9 | 496 | 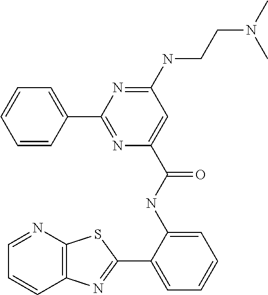 | A′ | A |
| 10 | 509 | 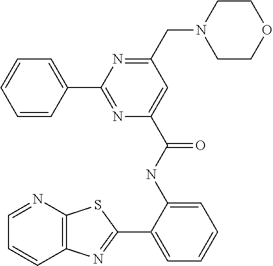 | A | A |
| 11 | 493 | 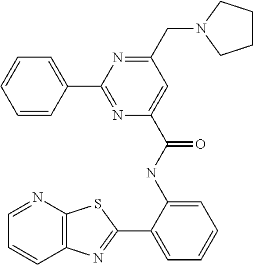 | B | A |
| 12 | 595 | 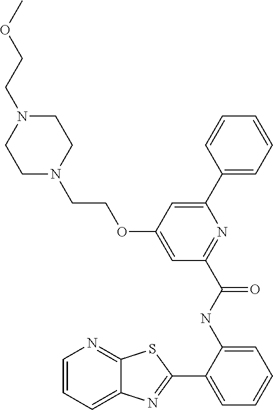 | A′ | A |
| 13 | 538 | 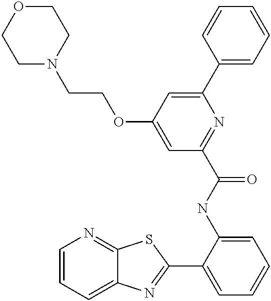 | A | A |
| 14 | 537 | 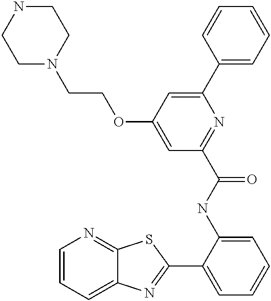 | A′ | A |
| 15 | 483 | 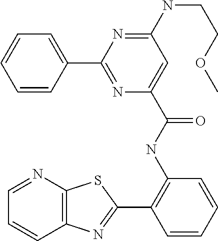 | A | A |
| 16 | 543 | 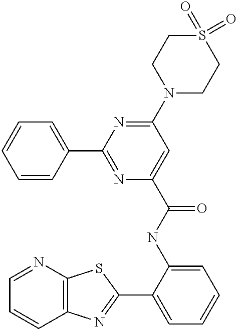 | A | B |
| 17 | 497 | 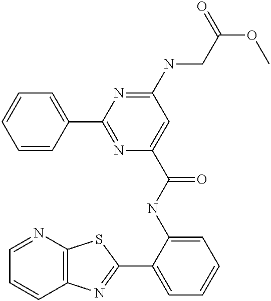 | A | A |
| 18 | 454 | 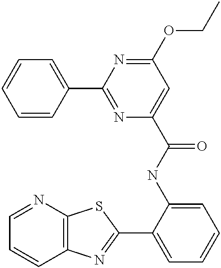 | C | C |
| 19 | 481 | 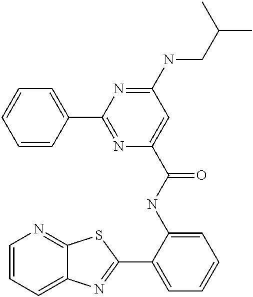 | C | C |
| 20 | 493 | 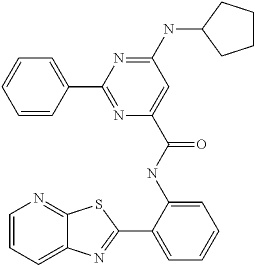 | C | C |
| 21 | 493 | 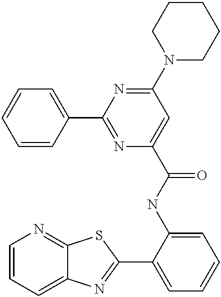 | C | C |
| 22 | 495 | 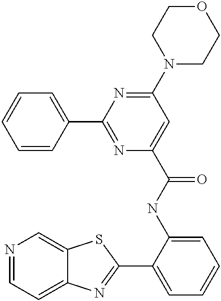 | A | A |
| 23 | 494 | 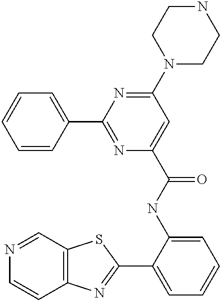 | A′ | A |
| 24 | 536 | 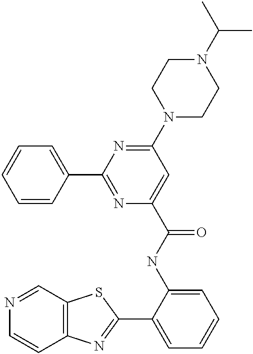 | A′ | A |
| 25 | 552 | 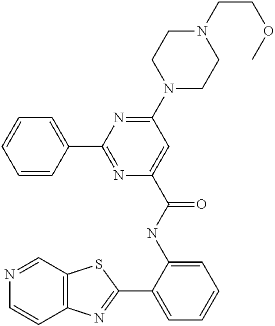 | A′ | A |
| 26 | 420 | 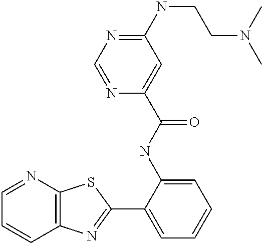 | B | A |
| 27 | 483 | 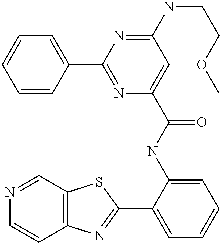 | A | A |
| 28 | 434 | 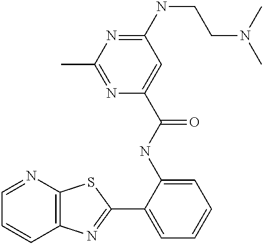 | B | A |
| 29 | 497 | 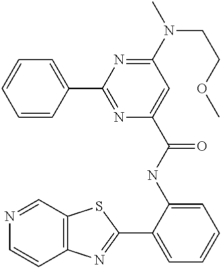 | A′ | A |
| 30 | 538 | 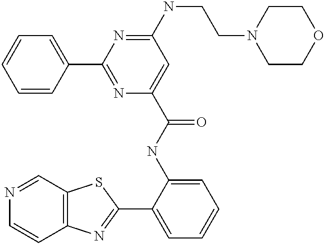 | A | A |
| 31 | 516 | 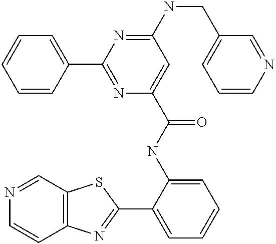 | A | B |
| 32 | 516 | 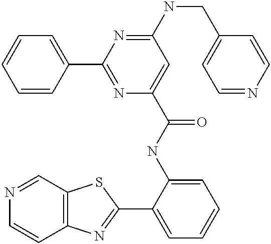 | A | A |
| 33 | 539 | 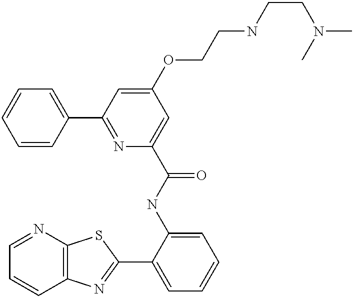 | A′ | A |
| 34 | 425 | 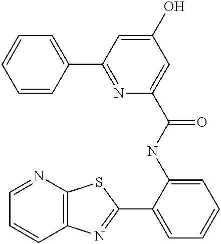 | A | A |
| 35 | 522 | 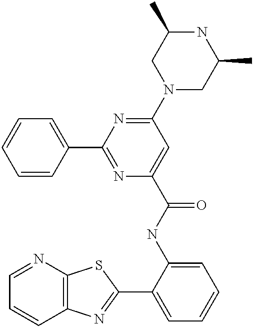 | A′ | A |
| 36 | 566 | 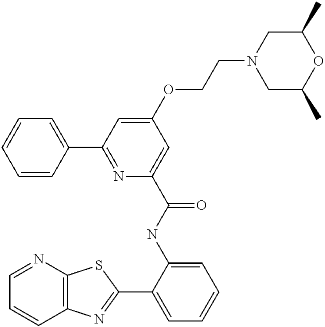 | A′ | A |
| 37 | 508 | 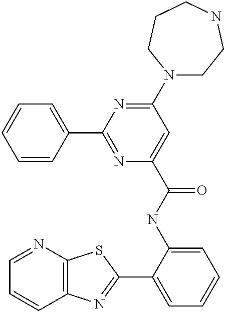 | A′ | A |
| 38 | 453 | 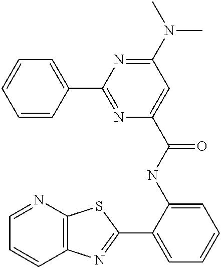 | B | B |
| 39 | 469 | 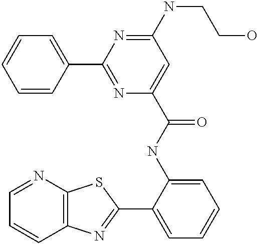 | A′ | A |
| 40 | 483 | 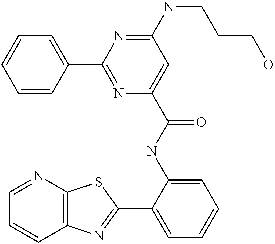 | A′ | A |
| 41 | 524 | 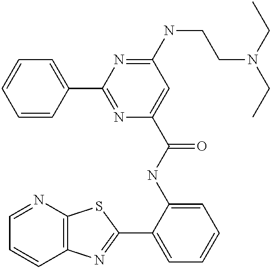 | A′ | A |
| 42 | 481 | 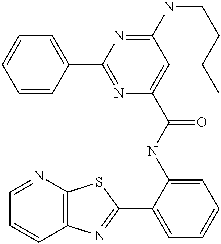 | C | C |
| 43 | 525 | 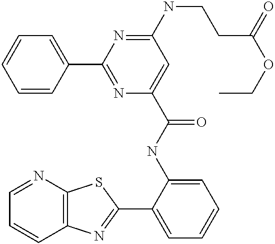 | A | B |
| 44 | 497 | 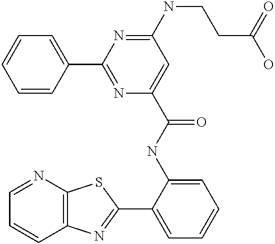 | A | A |
| 45 | 499 | 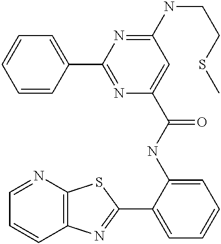 | B | B |
| 46 | 531 | 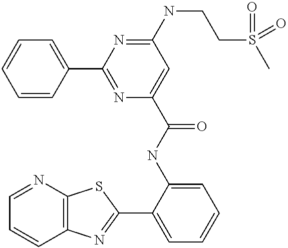 | A′ | A |
| 47 | 497 | 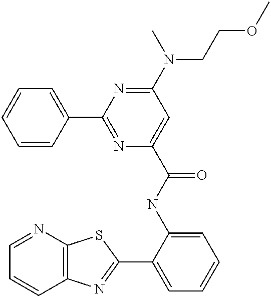 | A′ | A |
| 48 | 537 | 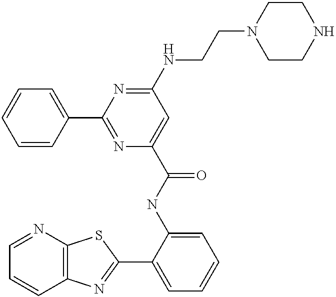 | A′ | A |
| 49 | 508 | 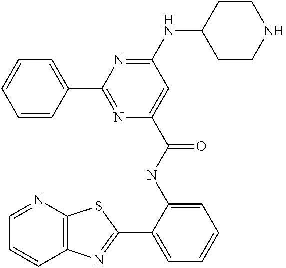 | A′ | A |
| 50 | 540 | 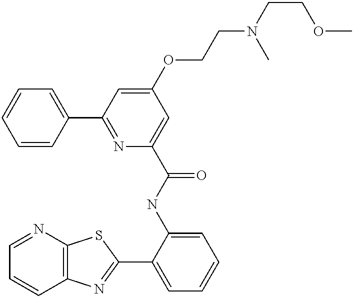 | A′ | A |
| 51 | 510 | 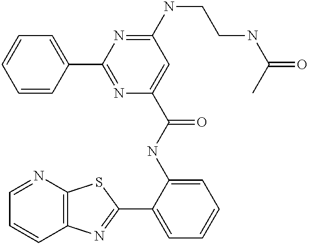 | A′ | A |
| 52 | 510 | 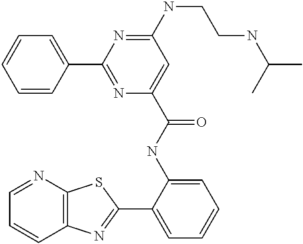 | A | A |
| 53 | 550 | 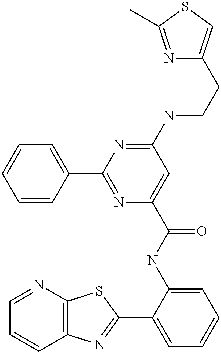 | A | B |
| 54 | 515 | 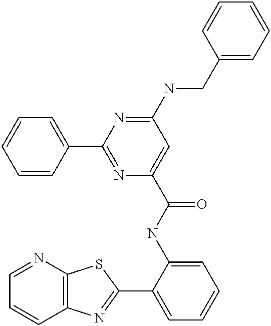 | C | C |
| 55 | 516 | 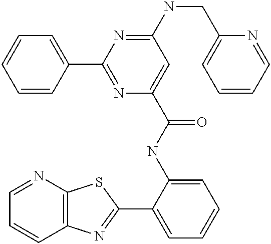 | A | B |
| 56 | 522 | 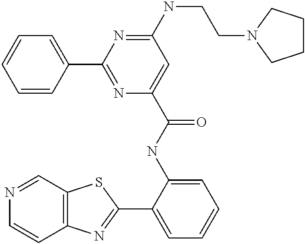 | A′ | A |
| 57 | 516 | 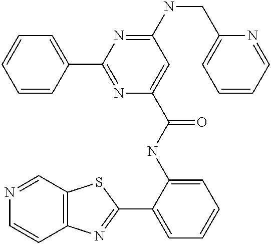 | A | A |
| 58 | 516 | 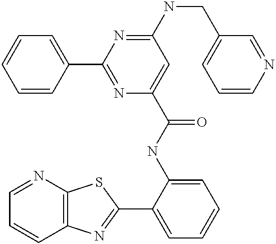 | A | A |
| 59 | 516 | 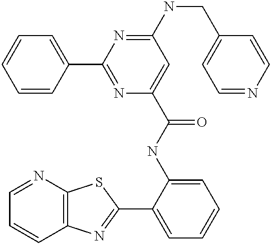 | A | A |
| 60 | 544 | 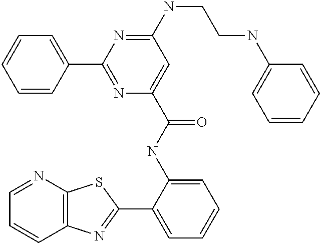 | C | C |
| 61 | 538 | 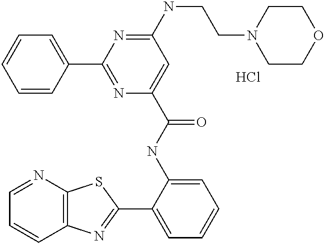 | A′ | A |
| 62 | 522 | 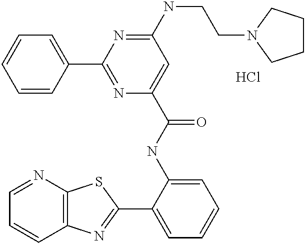 | A′ | A |
| 63 | 507 | 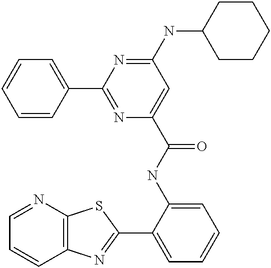 | C | C |
| 64 | 439 | 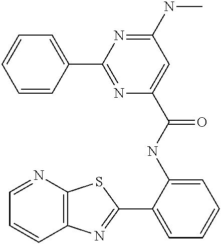 | C | C |
| 65 | 536 | 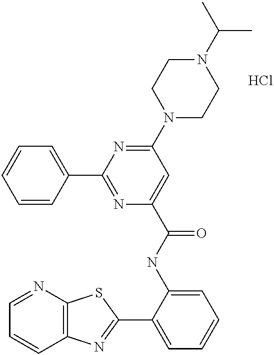 | A′ | B |
| 66 | 552 | 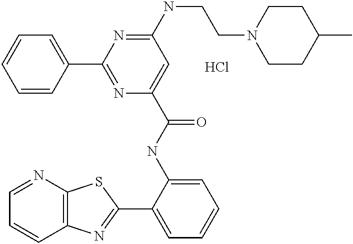 | A | A |
| 67 | 536 | 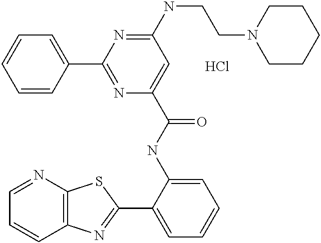 | A′ | A |
| 68 | 541 | 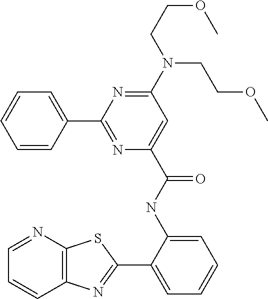 | A′ | A |
| 69 | 499 | 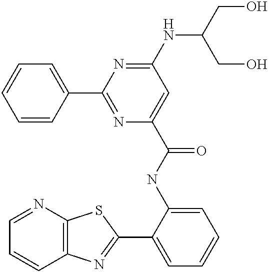 | A′ | A |
| 70 | 523 | 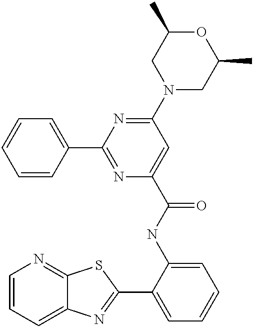 | A | B |
| 71 | 506 | 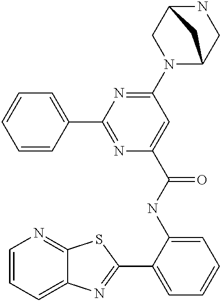 | A′ | A |
| 72 | 508 | 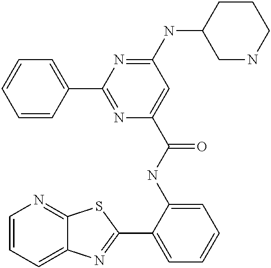 | A′ | A |
| 73 | 513 | 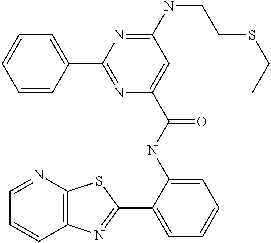 | B | B |
| 74 | 545 | 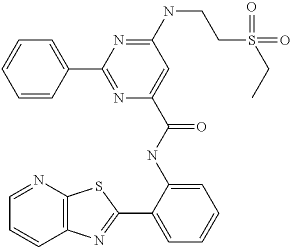 | A′ | A |
| 75 | 497 | 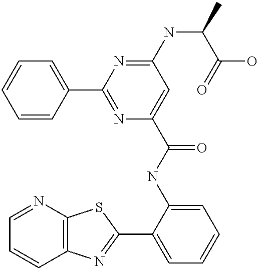 | A | A |
| 76 | 483 | 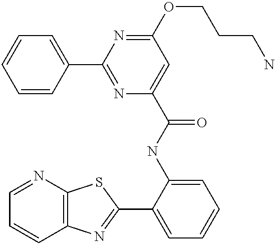 | A′ | A |
| 77 | 508 | 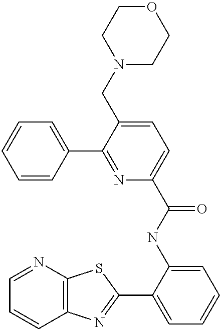 | A | B |
| 78 | 533 | 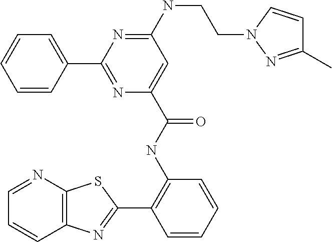 | A′ | A |
| 79 | 464 | 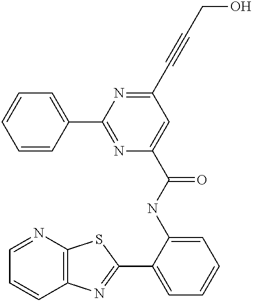 | C | C |
| 80 | 470 | 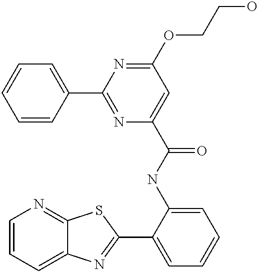 | C | C |
| 81 | 538 | 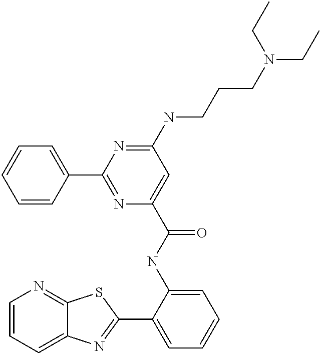 | A′ | A |
| 82 | 522 | 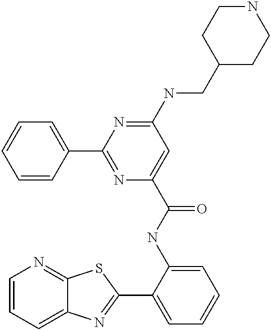 | A′ | A |
| 83 | 468 | 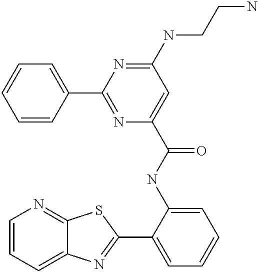 | A | A |
| 84 | 482 | 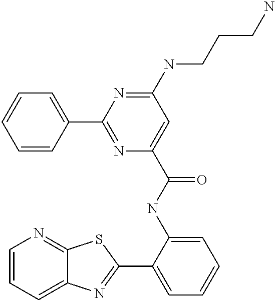 | A′ | A |
| 85 | 508 | 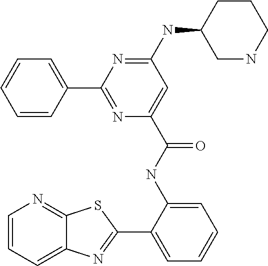 | A | A |
| 86 | 510 | 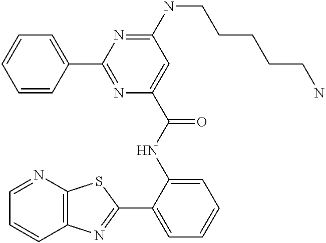 | A′ | A |
| 87 | 496 | 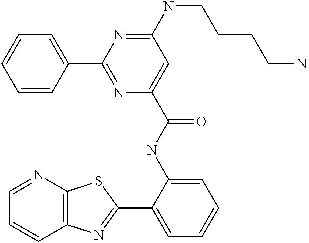 | A | A |
| 88 | 510 | 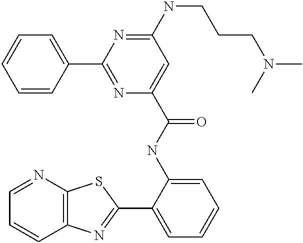 | A | A |
| 89 | 522 | 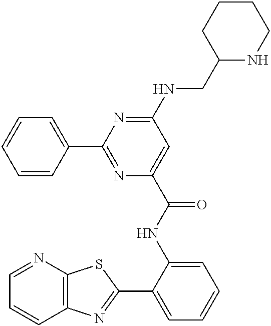 | A′ | A |
| 90 | 525 | 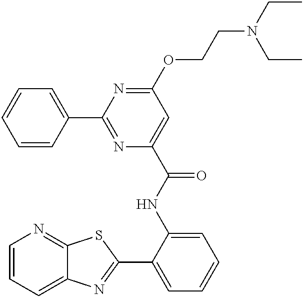 | A | A |
| 91 | 509 | 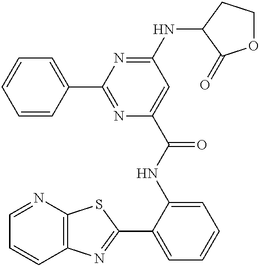 | A′ | A |
| 92 | 513 | 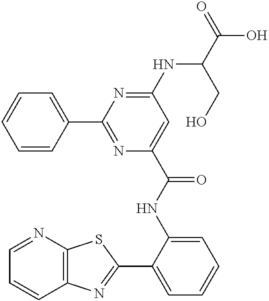 | B | A |
| 93 | 539 | 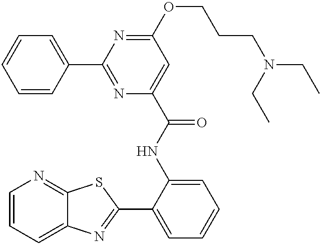 | A | A |
| 94 | 497 | 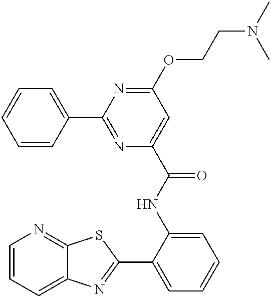 | A | A |
| 95 | 508 | 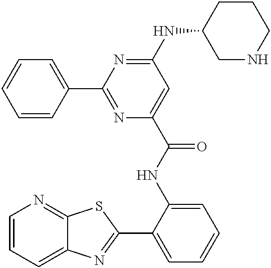 | A′ | A |
| 96 | 539 | 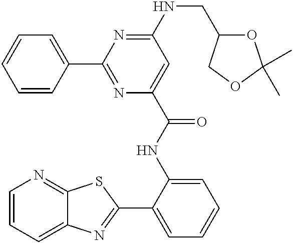 | A | A |
| 97 | 540 | 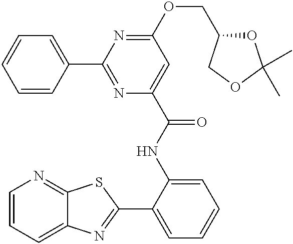 | C | B |
| 98 | 540 | 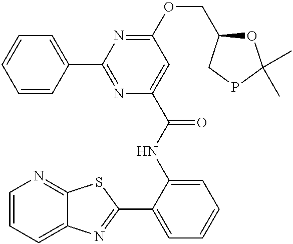 | C | C |
| 99 | 522 | 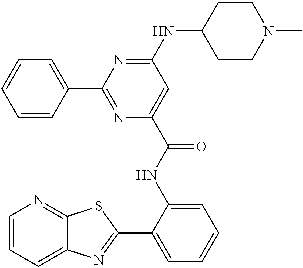 | A′ | A |
| 100 | 536 | 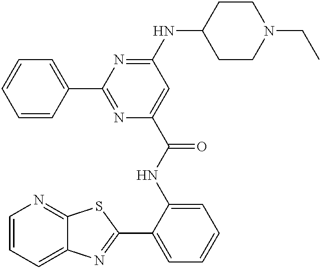 | A′ | A |
| 101 | 550 | 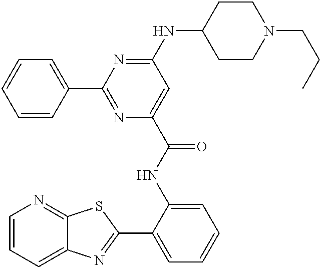 | A′ | A |
| 102 | 484 | 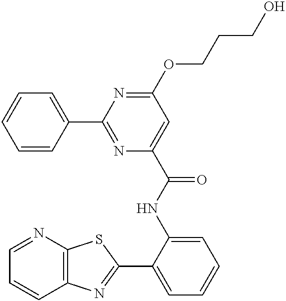 | A | B |
| 103 | 498 | 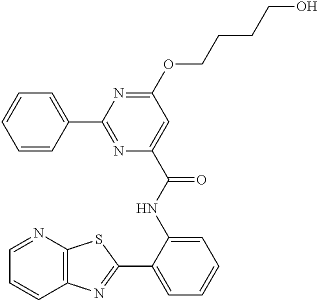 | C | B |
| 104 | 494 | 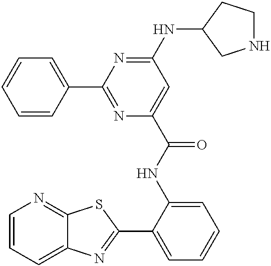 | A′ | A |
| 105 | 499 | 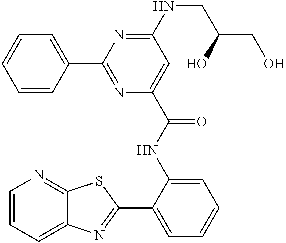 | A′ | A |
| 106 | 499 | 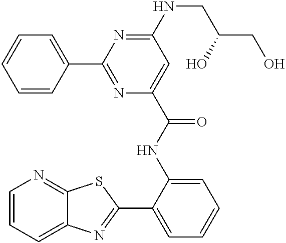 | A′ | A |
| 107 | 500 | 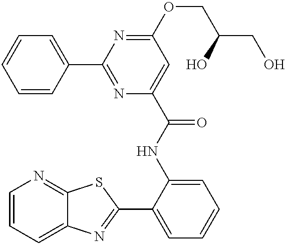 | A | B |
| 108 | 500 | 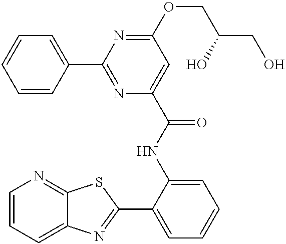 | A′ | B |
| 109 | 522 | 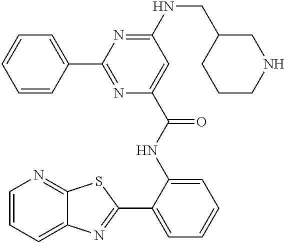 | A′ | A |
| 110 | 495 | 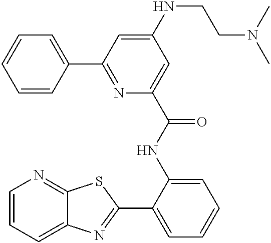 | A′ | A |
| 111 | 510 | 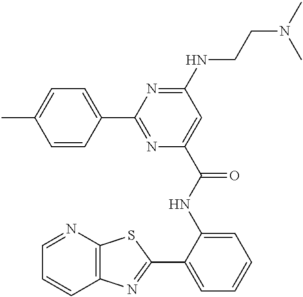 | A′ | A |
| 112 | 510 | 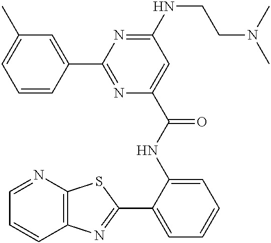 | A′ | A |
| 113 | 598 | 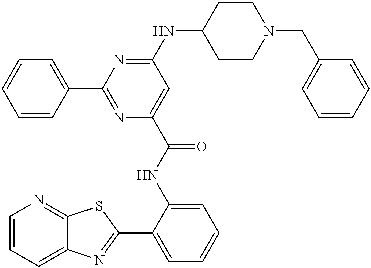 | A | B |
| 114 | 550 | 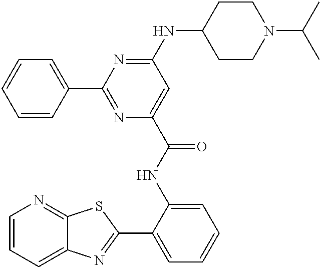 | A′ | A |
| 115 | 585 | 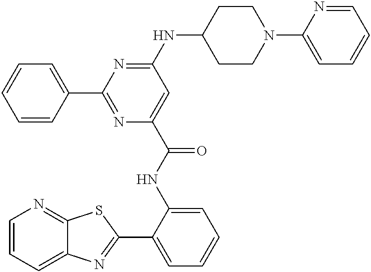 | C | C |
| 116 | 550 | 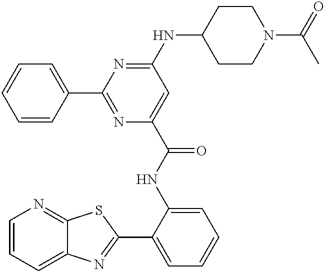 | A′ | A |
| 117 | 578 | 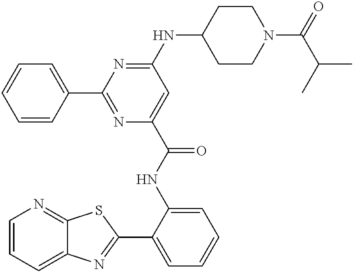 | A′ | A |
| 118 | 548 | 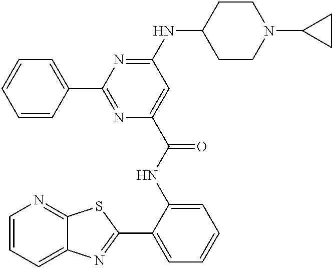 | A′ | A |
| 119 | 493 | 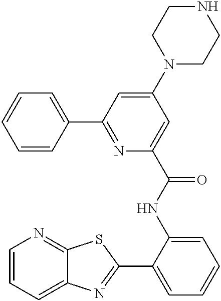 | A′ | A |
| 120 | 534 | 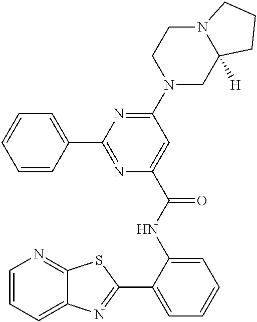 | A | A |
| 121 | 482 | 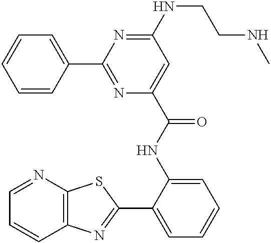 | A′ | A |
| 122 | 547 | 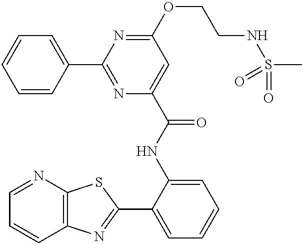 | A | B |
| 123 | 535 | 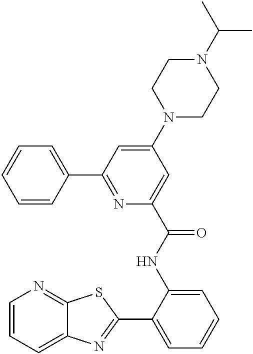 | A′ | A |
| 124 | 551 | 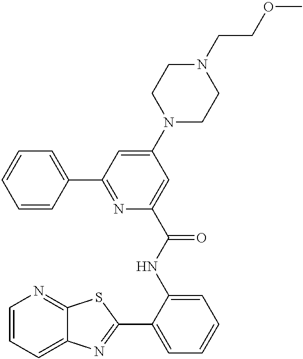 | A′ | A |
| 125 | 564 | 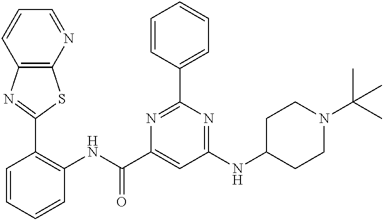 | A′ | A |
| 126 | 499 | 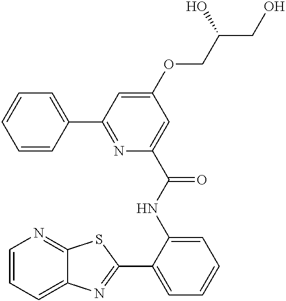 | A′ | A |
| 127 | 499 | 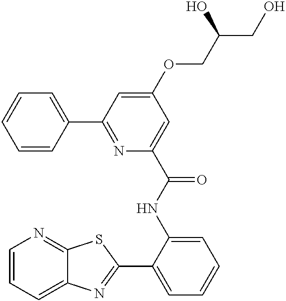 | A | A |
| 128 | 498 | 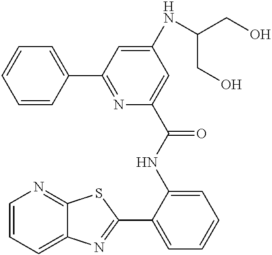 | A′ | A |
| 129 | 494 | 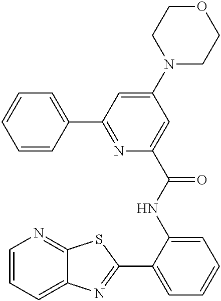 | A′ | A |
| 130 | 540 | 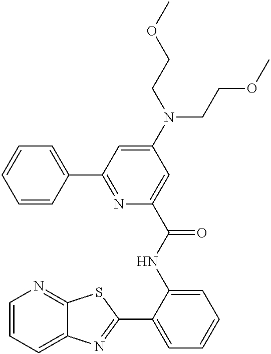 | A′ | A |
| 131 | 482 | 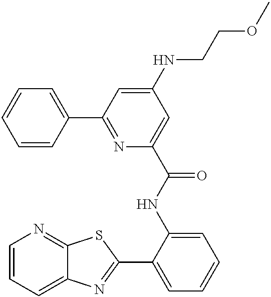 | A′ | A |
| 132 | 550 | 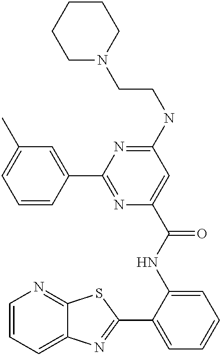 | A′ | A |
| 133 | 552 | 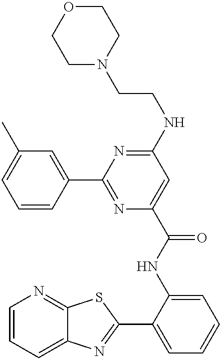 | A′ | A |
| 134 | 513 | 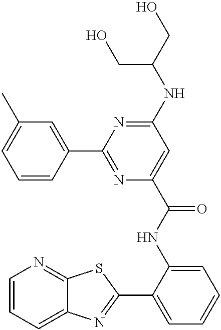 | A′ | A |
| 135 | 586 | 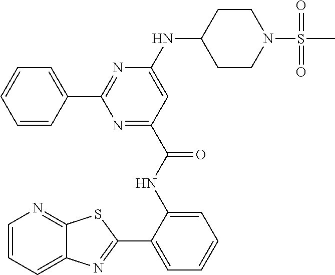 | A′ | A |
| 136 | 612 | 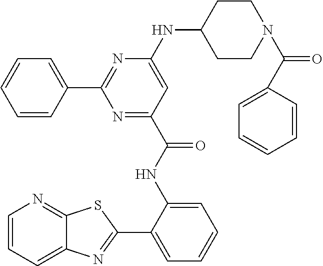 | A | B |
| 137 | 496 | 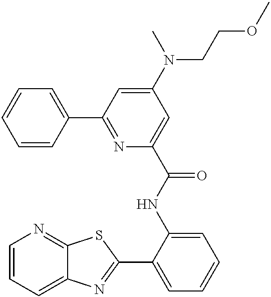 | A′ | A |
| 138 | 550 | 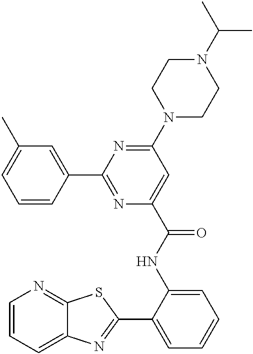 | A′ | A |
| 139 | 508 | 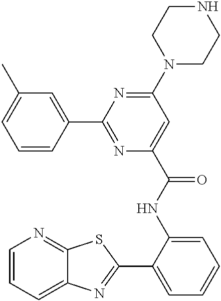 | A′ | A |
| 140 | 564 | 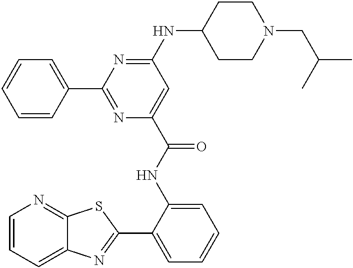 | A′ | A |
| 141 | 532 | 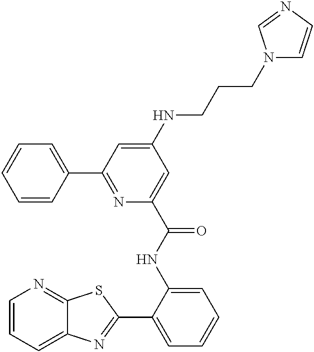 | A′ | A |
| 142 | 514 | 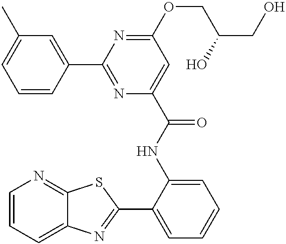 | A | A |
| 143 | 555 | 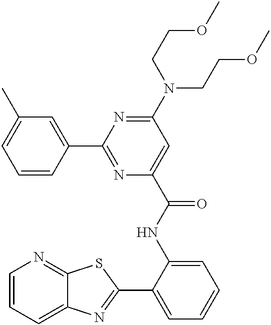 | A′ | A |
| 144 | 533 | 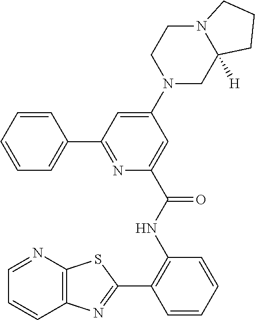 | A′ | A |
| 145 | 510 | 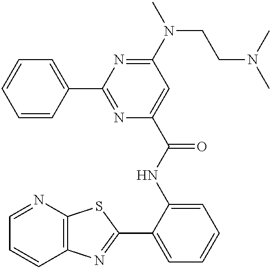 | A′ | A |
| 146 | 524 | 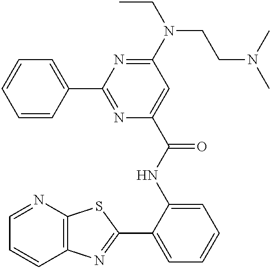 | A′ | A |
| 147 | 496 | 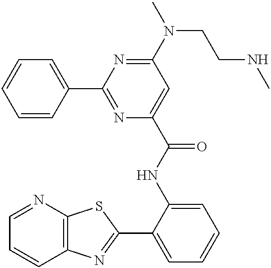 | A′ | A |
| 148 | 509 | 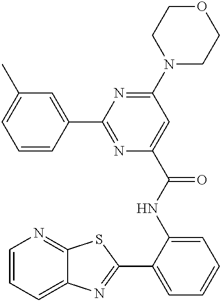 | A | B |
| 149 | 508 | 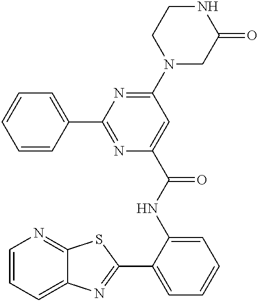 | A | B |
| 150 | 536 | 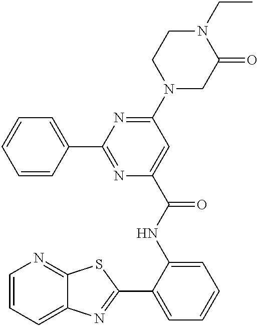 | A | A |
| 151 | 522 | 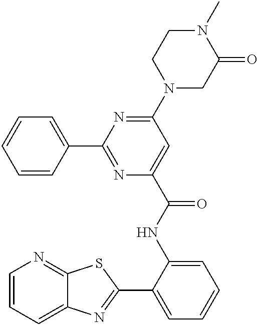 | A′ | B |
| 152 | 469 | 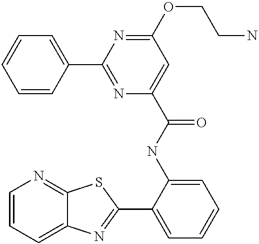 | A′ | A |
| 153 | 511 | 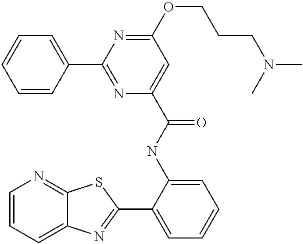 | A | A |

### EQUIVALENTS

The present invention provides among other things sirtuin-activating compounds and methods of use thereof. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: PCT Publications WO 2005/002672; 2005/002555; and 2004/016726.

1. A compound represented by Structural Formula (I): 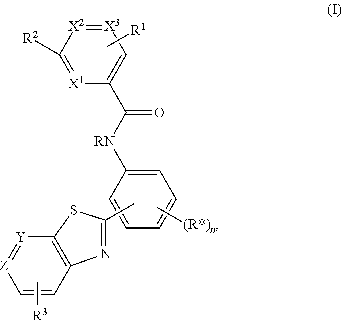
or a salt thereof, wherein:
two of X¹, X² and X³ are independently selected from —CH— and —N—;
the other of X¹, X² and X³ is —CH—;
R¹ is a solubilizing group;
R² is selected from phenyl, fluorophenyl and a 5- to 6-membered heterocycle containing an N heteroatom and, optionally, a second heteroatom selected from N, O or S, wherein said heterocycle is optionally substituted with methyl;
R is —H or —CH₃;
one of Y and Z is —CH— and the other of Y and Z is —N—;
R³ is selected from hydrogen, halo, lower alkyl, lower alkoxy, lower alkylthio and lower alkylsulfonyl;
R* is —CH₃ or a halogen; and
n is an integer from 0-4.

2. The compound of claim 1, wherein the structure is represented by Structural Formula (II):

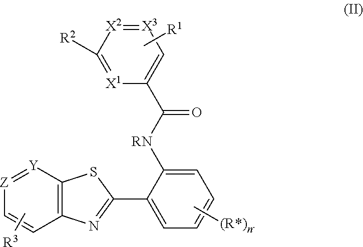

3. The compound of claim 2, wherein X¹ is —N—.

4. The compound of claim 3, wherein X¹ and X² are —N—.

5. The compound of claim 2, wherein Z is —N— and Y is —CH—.

6. The compound of claim 2, wherein Y is —N— and Z is —CH—.

7. The compound of claim 2, wherein R² is selected from phenyl, lower alkyl phenyl, fluorophenyl, methylthiazolyl, pyrimidinyl, pyridyl and pyrazolyl.

8. The compound of claim 7, wherein R² is phenyl.

9. The compound of claim 2, wherein R¹ is —NR⁴R⁵;
R⁴ is lower alkyl, monocyclyl amino, or monocyclyl lower alkyl; and
R⁵ is lower alkyl or H.

10. The compound of claim 9, wherein R⁴ is lower alkyl amino alkyl or lower dialkyl amino lower alkyl.

11. The compound of claim 2, wherein R¹ is a nitrogen-containing monocycle.

12. The compound of claim 11, wherein the point of attachment of the nitrogen-containing monocycle is an annular nitrogen.

13. The compound of claim 11, wherein R¹ is a 4, 5, 6, or 7-membered monocyclyl.

14. The compound of claim 13, wherein R¹ is represented by: 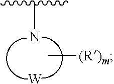
and the monocycle is a 5, 6 or 7-membered heterocycle;
W is —N(R⁶)—, —S(O₂)—, —C(R⁶R⁶)—, —N(CO₂R⁶)—, —O— or —S—;
R¹ in each occurrence is independently selected from H and lower alkyl;
m is 0 to 2; and
each R⁶ is independently selected from H and lower alkyl.

15. The compound of claim 2, wherein R¹ is represented by: 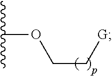
G is —NR⁴R⁵, —SR⁶, —OR⁶, —SO₂R⁶, —NCO₂R⁶ or monocyclyl;
R⁴ is lower alkyl, monocyclyl amino or monocyclyl lower alkyl and R⁵ is lower alkyl or H;
p is 0 to 3; and
each R⁶ is independently H or lower alkyl.

16. The compound of claim 2, wherein R¹ is —(CH₂)ₖG;
G is —NR⁴R⁵, —SR⁶, —OR⁶, —SO₂R⁶, —NCO₂R⁶ or monocyclyl;
R⁴ is lower alkyl, monocyclyl amino or monocyclyl lower alkyl and R⁵ is lower alkyl or H; and
k is 1 to 3;
each R⁶ is independently H or lower alkyl.

17. The compound of claim 2, wherein:
R² is selected from phenyl, 3-fluorophenyl and pyridyl;
R¹ is H; and
X¹ and X² are —N— and X³ is —CH—.

18. The compound of claim 17, wherein R¹ is —NR⁴R⁵; and
R⁴ is lower alkyl, monocyclyl amino or monocyclyl lower alkyl; and
R⁵ is lower alkyl or H.

19. The compound of claim 17, wherein R¹ is a nitrogen-containing monocycle wherein the point of attachment is an annular nitrogen.

20. The compound of claim 19, wherein R¹ is represented by: 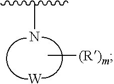
and the monocycle is a 5, 6 or 7-membered heterocycle;
W is —N(R⁶)—, —S(O₂)—, —C(R⁶R⁶)—, —N(CO₂R⁶)—, —O— or —S—;
R¹ in each occurrence is independently selected from H and lower alkyl;
m is 0 to 2; and
each R⁶ is independently selected from H and lower alkyl.

21. The compound of claim 17, wherein R¹ is represented by: 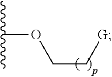
G is —NR⁴R⁵, —SR⁶, —OR⁶, —SO₂R⁶, —NCO₂R⁶ or monocyclyl;
R⁴ is lower alkyl, monocyclyl amino or monocyclyl lower alkyl and R⁵ is lower alkyl or H;
p is 0 to 3; and
each R⁶ is independently H or lower alkyl.

22. The compound of claim 17, wherein R¹ is —(CH₂)ₖG;
G is —NR⁴R⁵, —SR⁶, —OR⁶, —SO₂R⁶, —NCO₂R⁶ or monocyclyl;
R⁴ is lower alkyl, monocyclyl amino or monocyclyl lower alkyl and R⁵ is lower alkyl or H; and
k is 1 to 3;
each R⁶ is independently H or lower alkyl.

23. A pyrogen-free composition comprising a compound of claim 1, or a pharmaceutically acceptable salt thereof and a carrier.

24. A pharmaceutical composition comprising a compound of claim 1 and a pharmaceutically acceptable carrier.

25. The pharmaceutical composition of claim 24, further comprising an additional active agent.

26. A method for treating or preventing insulin resistance, a metabolic syndrome, diabetes, or complications thereof, or for increasing insulin sensitivity in a subject, comprising administering to the subject in need thereof a pharmaceutical composition of claim 24.

27. A compound represented by Structural Formula (I): 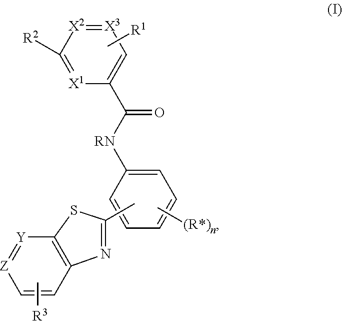
or a salt thereof, wherein:
two of X¹, X² and X³ are independently selected from —CH— and —N—;
the other of X¹, X² and X³ is —CH—;
R¹ is a solubilizing group;
R² is selected from phenyl, lower-alkyl phenyl, fluorophenyl and a 5- to 6-membered heterocycle containing an N heteroatom and, optionally, a second heteroatom selected from N, O or S, wherein said heterocycle is optionally substituted with methyl;
R is —H or —CH₃;
one of Y and Z is —CH— and the other of Y and Z is —N—;
R³ is selected from hydrogen, halo, lower alkyl, lower alkoxy, lower alkylthio and lower alkylsulfonyl;
R* is —CH₃ or a halogen; and
n is an integer from 0-4.
